(19) United States
(12) Patent Application Publication
(10) Pub. No.: US 2025/0381204 A1
(43) Pub. Date: Dec. 18, 2025

(54) A CANNABINOID MIXTURE WITH TERPENES FOR THE TREATMENT OF ANXIETY

(71) Applicant: TTS PHARMA LTD, Liverpool Merseyside (GB)

(72) Inventors: Mark TUCKER, Liverpool Merseyside (GB); Robert WALTON, Liverpool Merseyside (GB)

(73) Assignee: TTS PHARMA LTD, Liverpool Merseyside (GB)

(21) Appl. No.: 18/836,676

(22) PCT Filed: Feb. 7, 2023
(86) PCT No.: PCT/GB2023/050263

§ 371 Date: Aug. 7, 2024

(30) Foreign Application Priority Data
Feb. 8, 2022 (GB) 2201611.7

(51) Int. Cl. A61K 31/00; A61K 31/015; A61K 45/06; A61P 25/22

(52) U.S. Cl. CPC A61K 31/658; A61K 31/015; A61K 45/06; A61P 25/22

(57) ABSTRACT

There is disclosed a composition comprising a cannabinoid and at least one terpene, wherein the terpene:cannabinoid ratio is 1:3 to 1:60. The composition in total comprises ≤0.001% w/w of 9-tetrahydrocannabinol (THC) and <0.1% w/w of cannabigerol (CBG). There is also disclosed a composition comprising a cannabinoid and at least one terpene for use as a medicament. There is also disclosed a composition comprising a cannabinoid and at least one terpene for use in the treatment of anxiety and disorders associated with anxiety.

### CROSS-REFERENCE TO RELATED APPLICATIONS

The present application is the U.S. national stage application of International Application No. PCT/GB2023/050263, filed Feb. 7, 2023, which claims priority to GB Application No. 2201611.7, filed Feb. 8, 2022.

### FIELD OF INVENTION

The present invention provides a composition comprising a cannabinoid and the use of such a composition in medicine, in particular the treatment or prevention of anxiety or a disorder associated with anxiety, and the use of such a composition in a food supplement.

### BACKGROUND

Cannabinoid compositions, and the uses thereof, are a growing area of study and commercial activity.

Commercially available products include, for example, a botanical distillate, which is available as a 500 mg composition and a 1000 mg composition (www.budandtender.com/pages/500 mg-cbd-oil-laboratory-test-report and www.budandtender.com/pages/1000 mg-cbd-oil-laboratory-test-report, respectively). It is reported that the 500 mg composition comprises cannabidiol (CBD) and the following terpenes: alpha-pinene, beta-pinene, beta-myrcene, p-cymene, isopulegol, geraniol and beta-caryophyllene. The 1000 mg composition is reported as being similar to the 500 mg composition, except that it also contains D-limonene, linalool, guaiol, alpha-bisabolol, caryophyllene, and alpha-humulene. In both the 500 mg and 1000 mg compositions, no other terpenes were detected in a laboratory analysis.

Marx et al. (2018, Journal of Toxicology, Article ID 8143582) provides a supercritical CO₂ extract of the aerial parts of the Cannabis sativa plant, which is 26% w/w phytocannabinoids and 61% edible fatty acids, with the remaining 13% including fatty alkanes, plant sterols, triterpenes and tocopherols. Marx et al. does not report the concentration of terpenes in the extract. The aim of this study was to perform toxicological studies on the extract, and no uses of the extract are disclosed.

Radmila et al. (2018, Molecules, 23, 1230) provides an analysis of 15 commercially available CBD oils. Of these, Oil 3 was found to comprise 0.79% w/w (3247 μg/g) CBD, 16 μg/g cannabigerol (CBG), 174.05 μg/g total terpenes and 148 μg/g tetrahydrocannabinol (THC), with the carrier oil being olive oil. Oil 12 was found to comprise 1.61% w/w (12,758 μg/g) CBD, 6 μg/g CBG, 981.37 μg/g total terpenes and 494 μg/g THC, with the carrier oil being hemp seed oil. Oil 14 was found to comprise 3.09% w/w (23,186 μg/g) CBD, 460 μg/g CBG, 752.82 μg/g total terpenes and 524 μg/g THC, with the carrier oil being hemp seed oil.

With regard to the uses of cannabinoid compositions, there is a report of a cannabinoid composition for treating teenagers with social anxiety disorders (Masataka et al. 2019; Front. Psychol. 10:2466). The composition used was RSHO-X Hemp Oil (the product of HempMeds, USA). According to the study, a 236 ml bottle of the product contained 5,000 mg of CBD, but no delta-9-tetrahydrocannabinol (THC), or any other cannabinoids or terpenes.

US 2020/0261404 discloses a composition comprising at least one cannabinoid, at least one primary terpene and at least 5% by weight of a non-cannabinoid, non-terpene carrier. The at least one cannabinoid may be THC, THCA, CBD, CBDA, CBG, CBGA, CBC, CBCA, THCV, THCVA, CBDV, CBDVA, CBN, CBNA, CBL or CBLA.

WO 2018/173049 discloses a vaporizable composition comprising 1-30 wt % cannabinol (CBN) and up to 15 wt % of at least one terpene. The composition may contain additional cannabinoids, and is for the purpose of treating sleep disorders such as insomnia.

Despite the existence of the cannabinoid compositions mentioned above, there remains a need for new cannabinoid compositions, especially new cannabinoid compositions with improved activity.

### SUMMARY OF INVENTION

The present invention arises from the surprising finding that a cannabinoid composition containing at least one terpene, wherein the terpene:cannabinoid ratio is 1:3 to 1:60 w/w is particularly effective in the treatment of anxiety and disorders associated with anxiety. Disclosed herein is a composition comprising a cannabinoid selected from cannabidiol (CBD), cannabigerol (CBG) and a mixture thereof, and at least one terpene.

In one aspect of the invention, the composition comprises a cannabinoid and at least one terpene, wherein the cannabinoid comprises cannabidiol (CBD), the terpene:cannabinoid ratio by weight is from 1:3 to 1:60 w/w and the composition in total comprises ≤0.001% w/w of 9-tetrahydrocannabinol (THC) and <0.1% w/w of cannabigerol (CBG).

Preferably, the terpene:cannabinoid ratio by weight is 1:3 to 1:40 w/w, 1:3 to 1:35 w/w, 1:3 to 1:20 w/w, 1:5 to 1:20 w/w, 1:5 to 1:9 w/w, 1:5 to 1:7 w/w, 1:7 to 1:12 w/w, 1:9 to 1:20 w/w, or 1:15 to 1:20 w/w.

Advantageously, CBG is at a concentration of ≤0.01% w/w or more preferably ≤0.001% w/w.

Preferably, the composition does not comprise CBG.

Advantageously, the composition in total comprises ≤0.001% w/w of 9-tetrahydrocannabinol (THC).

Preferably, the at least one terpene comprises one or more of terpinolene, beta myrcene and beta pinene.

Advantageously, the at least one terpene comprises alpha-humulene, beta-caryophyllene, alpha-pinene, beta pinene, terpinolene, beta myrcene and/or an ocimene isomer. Conveniently, the cannabinoid is present at a concentration of 0.03-30% w/w, preferably 1-25% w/w, more preferably 5-25% w/w.

Preferably, the at least one terpene is present at a concentration of 0.01-10% w/w, preferably 0.1-6% w/w, more preferably 0.5-3% w/w.

In some embodiments, the composition comprises CBD and CBG.

Conveniently, the composition comprises ≤0.0001% w/w of cannabinol (CBN), 9-tetrahydrocannabinol (THC), and/or 9-tetrahydrocannabinolic acid (THC-A). Advantageously, the composition comprises ≤0.0001% w/w of cannabichromene (CBC) and cannabidiolic acid (CBD-A).

Preferably, the composition does not comprise THC.

Preferably, the composition further comprises an oil.

Advantageously, the cannabinoid:oil ratio is 1:2 to 1:3500 w/w.

Alternatively, the oil is present at 60-99.96% w/w.

Advantageously, the oil is selected from hemp seed oil, rape seed oil, coconut oil, olive oil, cranberry oil, vegetable oil and MCT oil, or a mixture or two or more oils selected from hemp seed oil, rape seed oil, coconut oil, olive oil, cranberry oil and MCT oil.

In a second aspect of the invention, there is provided a pharmaceutical formulation comprising the composition according to the first aspect and a pharmaceutically-acceptable diluent, excipient or carrier.

In a third aspect of the invention, there is provided the composition according to the first aspect or the pharmaceutical formulation according to the second aspect for use as a medicament. Preferably, the composition or pharmaceutical formulation is for use in the treatment or prevention of anxiety or a disorder associated with anxiety.

Advantageously, the composition or pharmaceutical formulation is for use in the treatment or prevention of generalized anxiety disorder, chronic anxiety, social anxiety disorder, panic disorder or episodic paroxysmal anxiety, obsessive compulsive disorder, post-traumatic stress disorder, phobic anxiety disorders, social phobias, specific phobias such as agoraphobia, claustrophobia or animal phobias, acute stress disorder, separation anxiety disorder, selective mutism, substance or medication-induced anxiety disorder, anxiety disorders due to other medical conditions, anxiety disorders without a specific cause, depression, atypical depression, recurring subclinical anxiety, persistent anxiety, chronic subclinical anxiety, persistent anxiety, anxious depression, neurosis, healing avoidance anxiety, dissociative anxiety, mixed anxiety and depressive disorder, severe stress, adjustment disorders, dissociative disorders such as dissociative amnesia, dissociative fugue, dissociative stupor, trance and possession disorders, dissociative motor disorders, dissociative convulsions, dissociative anaesthesia and sensory loss, mixed dissociative disorders, Ganser syndrome, multiple personality disorder and dissociative conversion disorder, somatoform disorders such as somatization disorder, undifferentiated somatoform disorder, hypochondriacal disorder, somatoform autonomic dysfunction and persistent somatoform pain disorder, and neurotic disorders such as neurasthenia, depersonalization-derealization syndrome, Dhat syndrome, occupational neurosis, psychasthenia, psychasthenic neurosis and psychogenic syncope.

In a fourth aspect of the invention, there is provided a food or beverage product comprising the composition of the invention.

In a fifth aspect of the invention, there is provided the use of the composition according to the first aspect or the pharmaceutical formulation according to the second aspect for the treatment or prevention of non-clinical anxiety.

In a sixth aspect of the invention, there is provided a method of treating or preventing anxiety, wherein the method comprises administering the composition according to the first aspect or the pharmaceutical formulation according to the second aspect to a patient in need thereof.

In a seventh aspect of the invention, there is provided the use of the composition according to the first aspect for the manufacture of a medicament for the treatment or prevention of anxiety. In this specification the term “cannabinoid” means chemicals that are found in Cannabis plants. The term includes cannabinoids found in plants other than Cannabis plants, such as Echinacea purpurea, Echinacea angustifolia, Acemila oleracea, Helichrysum umbraculigerum and Radula marginata. The term includes both phytocannabinoids and synthetic cannabinoids. It includes the classes of cannabidiol (CBD) and cannabigerol (CBG). The term “cannabinoid” also includes the classes of cannabichromene, cannabicyclol, cannabivarin, tetrahydrocannabivarin, cannabidivarin, cannabichromevarin, cannabigerovarin, cannabigerol monomethyl ether, cannabielsoin and cannabicitran. The term “cannabinoid” also covers modified versions of the naturally occurring cannabinoids which retain at least 20% of the activity. The term “cannabinoid” also includes controlled cannabinoids such as trans-delta-9-tetrahydrocannabinol-C5, Cis-delta-9-tetrahydrocannabinol-C5, Delta-9-tetrahydrocannabinol-C4, Delta-9-tetrahydrocannabinol-C3 (Delta-9-tetrahydrocannabivarin), Delta-9-tetrahydrocannabinol-C1, Delta-8-tetrahydrocannabinol, Cannabinol-C1, Cannabinol-C2, Cannabinol-C3, Cannabinol-C4, Cannabinol-C5 and Cannabinol methyl ether-C5.

In this specification the term “treatment” means complete cure of a clinical or non-clinical condition as well as partially alleviating the symptoms thereof but without complete cure of the condition. It also refers to both short-term alleviating of symptoms as well as long term alleviating of symptoms.

In this specification the term “prevention” means complete prevention of a clinical or non-clinical condition as well as the partial prevention of symptoms thereof but without complete prevention of the condition. It also refers to both short-term prevention of symptoms as well as long-term prevention of symptoms.

In this specification the term “terpene:cannabinoid ratio” is a ratio of the total amount (w/w) of all terpenes present in the composition to the total amount (w/w) of cannabinoids present in the composition. However, it will be appreciated that in some embodiments of the present invention, the “terpene:cannabinoid ratio” refers to the ratio of the total amount (w/w) of the following terpenes present in the composition (alpha-humulene, beta-caryophyllene, alpha-pinene, beta-pinene, terpinolene, beta myrcene and trans beta ocimene) to the total amount (w/w) of cannabinoids present in the composition. Similarly, the term “terpene:CBD ratio” is a ratio of the total amount (w/w) of all terpenes present in the composition to the total amount (w/w) of CBD present in the composition. However, it will be appreciated that, in some embodiments, the “terpene:CBD ratio” refers to the ratio of the total amount (w/w) of the following terpenes present in the composition: beta myrcene, beta caryophyllene, terpinolene, alpha-pinene, alpha-humulene, trans beta ocimene, and beta-pinene, to the total amount (w/w) of CBD present in the composition.

In this specification the term “cannabinoid:oil ratio” is a ratio of the total amount (w/w) of cannabinoids present in the composition to the total amount (w/w) of carrier oils present in the composition. Similarly, the term “CBD:oil ratio” is a ratio of the total amount (w/w) of CBD present in the composition to the total amount (w/w) of carrier oils present in the composition.

In this specification the term “ocimene isomer” refers to any of alpha-ocimene, trans beta-ocimene and cis beta-ocimene.

### BRIEF DESCRIPTION OF THE FIGURES

FIG. 1A shows the mean distance travelled (mm) per 10 seconds for Experiment 1, trial 1 of Example 1.

FIG. 1B shows the mean distance travelled (mm) per 10 seconds for Experiment 1, trial 2 of Example 1.

FIG. 2 shows the mean distance travelled (mm) per 10 seconds for Experiment 2 of Example 1.

FIG. 3 shows the mean distance travelled (mm) per 10 seconds (test product). Dose 1=0.1 mg/L test product, Dose 2=0.5 mg/L test product, Dose 3=1 mg/L test product, Dose 4=5 mg/L test product, Dose 5=10 mg/L test product and Dose 6=20 mg/L test product.

FIG. 4 shows the mean distance travelled (mm) per 10 seconds (Fraction 1 in methanol). Result 1=control (fish water), Result 2=0.001% methanol, Result 3=0.5 mg/L Fraction 1, Result 4=1 mg/L Fraction 1, Result 5=5 mg/L Fraction 1, Result 6=10 mg/L Fraction 1, Result 7=15 mg/L Fraction 1, Result 8=20 mg/L Fraction 1.

FIG. 5 shows the mean distance travelled (mm) per 10 seconds (Fraction 1 in DMSO).

FIG. 6 shows the mean distance travelled (mm) per 10 seconds (Fraction 1 in methanol vs. DMSO vs. test product). Control, 0.001 DMSO, 0.001 methanol, Dose 5=10 mg/L test product, Dose 12=20 mg/L Fraction 1 in methanol, Dose 18=20 mg/L Fraction 1 in DMSO, Dose 10=10 mg/L Fraction 1 in methanol and Dose 16=10 mg/L Fraction 1 in DMSO.

FIG. 7 shows the mean distance travelled (mm) per 10 seconds for Experiment 1 of Example 3. Dose 1 to Dose 6:0.1 mg/L, 0.5 mg/L, 1 mg/L, 5 mg/L, 10 mg/L, 20 mg/L (40 mg/L killed the larvae). Arrows indicate light periods (locomotion decreases when the light comes on). Mean+/−SEM. N=6.

FIG. 8 shows the baseline comparison for Experiment 1 of Example 3 (Fraction 1 in methanol).

FIG. 9 shows the mean distance travelled over the last 60 seconds of the baseline period for Experiment 1 of Example 3.

FIG. 10 shows the slope of the mean distance/time regression for each individual per condition in each light number. For each light event number, from left to right: Control, 0.001 meoh, 0.1 mg/L, 0.5 mg/L, 1 mg/L, 5 mg/L, 10 mg/L and 20 mg/L.

FIG. 11 shows the mean distance travelled (mm) per 10 seconds. Results 1 to 8: control, 0.001% methanol, 5 mg/L, 1 mg/L, 5 mg/L, 10 mg/L, 15 mg/L and 20 mg/L of Fraction 1.

FIG. 12 shows the Dose Response curve for last minute of baseline period for Experiment 2 (the mean distance travelled over the last 60 seconds of the baseline period).

FIG. 13 shows the mean distance travelled (mm) per 10 seconds across the experimental time course. Results: control, 0.001% DMSO, dose 13-18=0.5 mg/L, 1 mg/L, 10 mg/L, 15 mg/L and 20 mg/L of Fraction 1.

FIG. 14 shows the Dose Response curve for last minute of baseline period for Experiment 3 (the mean distance travelled over the last 60 seconds of the baseline period).

FIG. 15 shows the mean distance travelled (mm) per 10 seconds across the experimental time course. Results control, 0.001 DMSO, 0.001 methanol, Dose 5=10 mg/L test product in DMSO, Dose 12=20 mg/L Fraction 1 in methanol, Dose 18=20 mg/L Fraction 1 in DMSO, Dose 10=10 mg/L Fraction 1 in methanol, Dose 16=10 mg/L Fraction 1 in DMSO.

FIG. 16 shows the mean distance travelled over the last 60 seconds of the baseline period for Experiment 4 of Example 3.

FIG. 17 shows the mean distance travelled (mm) per 10 seconds across the experimental time course. Result 1 is control, Result 2 is methanol, Result 3 is 0.5 mg/L, Result 4 is 5 mg/L, Result 5 is 10 mg/L and Result 6 is 20 mg/L test product.

FIG. 18 shows the Dose Response curve for the last minute of the baseline period.

FIG. 19 shows the mean distance travelled (mm) per 10 seconds across the experimental time course. Control, carrier=0.001% DMSO, test product concentration range 0.12-48 mg/L.

FIG. 20 shows the Dose Response curve for the last minute of the baseline period.

FIG. 21 shows the mean distance travelled (mm) per 10 seconds across the experimental time course. Control (fish water), carrier=0.001% DMSO, fraction 2 concentration range 0.12-48 mg/L.

FIG. 22 shows the Dose Response curve for the last minute of the baseline period.

FIG. 23 shows the mean distance travelled (mm) per 10 seconds across the experimental time course (Fraction 2). Results=control (fish water), 0.002% methanol, 0.5 mg/L, 1.0, 2.0, 5.0 mg/L, 10 mg/L and 20 mg/L.

FIG. 24 shows the mean distance travelled (mm) per 10 seconds across the experimental time course (Fraction 2). Results=control (fish water), 0.002% DMSO, 0.5 mg/L, 1.0, 2.0, 5.0 mg/L, 10 mg/L and 20 mg/L.

FIG. 25 shows the mean distance travelled (mm) per 10 seconds across the experimental time course (Fraction 3). Results=control (fish water), 0.001% methanol, 0.5 mg/L, 1.0, 2.0, 5.0 mg/L, 10 mg/L and 20 mg/L.

FIG. 26 shows the mean distance travelled (mm) per 10 seconds across the experimental time course (Fraction 3). Results=control (fish water), 0.001% DMSO, 0.5 mg/L, 1.0, 2.0, 5.0 mg/L, 10 mg/L and 20 mg/L.

FIG. 27 shows the mean distanced travelled (mm) per 10 seconds across the experimental time course (Fraction 1).

FIG. 28 shows the dose response curve (Fraction 1).

FIG. 29 shows the mean distanced travelled (mm) per 10 seconds across the experimental time course (Fraction 2).

FIG. 30 shows the dose response curve (Fraction 2)

FIG. 31 shows the mean distanced travelled (mm) per 10 seconds across the experimental time course (Fraction 3).

FIG. 32 shows the dose response curve (Fraction 3).

FIG. 33 shows the mean distance travelled (mm) per 10 seconds across the experimental time course (control vs. DMSO).

FIG. 34 shows the mean distanced travelled (mm) per 10 seconds across the experimental time course (test product).

FIG. 35 shows the dose response curve for the test product.

FIG. 36 shows the mean distanced travelled (mm) per 10 seconds across the experimental time course (diazepam).

FIG. 37 shows the dose response curve for diazepam.

FIG. 38 shows the mean distanced travelled (mm) per 10 seconds across the experimental time course (Ethanol).

FIG. 39 shows the dose response curve for Ethanol.

FIG. 40 shows the mean distanced travelled (mm) per 10 seconds across the experimental time course (4 dpf vs. 5 dpf larvae).

FIG. 41 shows the mean distanced travelled (mm) per 10 seconds across the experimental time course (Caffeine).

FIG. 42A shows the dose response curve (Caffeine).

FIG. 42B shows the distance moved at baseline (Caffeine).

FIG. 43 shows the mean distance travelled (mm) per 10 seconds over the whole duration of the assay (diazepam).

FIG. 44A shows the dose response curve (diazepam).

FIG. 44B shows the distance moved at baseline (diazepam).

FIG. 45 shows the mean distance moved (mm) during the dark period (diazepam). From top to bottom, relative to the starting point of the line at the left-hand y axis: 2.5 μM, 0.25 μM, 0.01 μM, CTRL, 0.1 μM and 0.025 μM.

FIG. 46 shows the mean distance moved (mm) during the light challenge (diazepam). From top to bottom, relative to the starting point of the line at the left-hand y axis: 2.5 μM, 0.025 μM, 0.1 μM, 0.25 μM, CTRL, 0.01 μM.

FIG. 47 shows the mean distance moved (mm) during the baseline period (diazepam).

FIG. 48 shows the mean distance travelled (mm) per 10 seconds over the whole duration of the assay (Fraction 1).

FIG. 49 shows the dose response curve (Fraction 1).

FIG. 50 shows the mean distance travelled (mm) per 10 seconds over the whole duration of the assay (Fraction 2).

FIG. 51 shows the dose response curve (Fraction 2).

FIG. 52 shows the mean distance travelled (mm) per 10 seconds over the whole duration of the assay (Fraction 3).

FIG. 53 shows the dose response curve and distance moved at baseline (Fraction 3).

FIG. 54 shows the mean distance travelled (mm) per 10 seconds over the whole duration of the assay (test product).

FIG. 55 shows the dose response curve (test product).

FIG. 56—Comparing basal locomotion of different drugs. Mean distance moved (mm) was measured in the last minute of the baseline period. A) shows basal movement of ethanol, fluoxetine, caffeine and NaCl compared to DMSO in the last minute of the baseline period. B) shows basal movement of increasing concentrations of test product compared to DMSO in the last minute of the baseline period. C) shows basal movement of increasing concentrations of Fraction 1 compared to DMSO in the last minute of the baseline period. D) shows basal movement of increasing concentrations of Fraction 2 compared to DMSO in the last minute of the baseline period. A: Ethanol was found to move significantly more (p<0.001). Fluoxetine and caffeine were found to move significantly less (p<0.001, p<0.05). B: There was no significant difference found in basal locomotion in concentrations of test product (p>0.05). C: All concentrations of fraction 1 with the exception of 5.0 mg/L were found to move significantly more (p<0.01). D: There were significant differences found in basal movement in Fraction 2 concentrations 5.0 mg/L, 7.5 mg/L and 10 mg/L (p<0.05, p<0.01, p<0.001, respectively), see fig D. Data (Mean±S.E.M), p>0.05; by linear mixed model (LMM). Replicates; DMSO n=70, Ethanol n=54, Fluoxetine n=53, Caffeine n=38, NaCl n=38, test product; 10 mg/L n=37, 12.5 mg/L n=36, 15 mg/L n=36. Fraction 1; 2.5 mg/L n=35, 5.0 mg/L n=36, 7.5 mg/L n=36, 10 mg/L n=36. Fraction 2; 2.5 mg/L n=35, 5.0 mg/L n=36, 7.5 mg/L n=36, 10 mg/L n=35.

FIG. 57—Comparing different compounds to DMSO in the light challenge of the forced light/dark transition. Mean distance moved (mm) was measured every 10 s during the light challenge, lasting 10 min. A) Shows mean distance moved (mm) over time of Ethanol, Fluoxetine, Caffeine and NaCl compared to DMSO over a period of 10 min in the light. B) Shows mean distance moved (mm) over time of increasing dosages of test product compared to DMSO over a period of 10 min in the light. C) Shows mean distance moved (mm) over time of increasing dosages of Fraction 1 compared to DMSO over a period of 10 min in the light. D) Shows mean distance moved (mm) over time of increasing dosages of Fraction 2 compared to DMSO over a period of 10 min in the light. A: Ethanol and caffeine have a negative relation with time (p<0.001) and froze less (p<0.001). Fluoxetine recovered less (p<0.001). NaCl did not recover differently from DMSO, however froze less (p<0.05). B: 10 mg/L and 12.5 mg/L test product recover faster compared to DMSO (p<0.05), 15 mg/L does not recover faster. 10 mg/L and 15 mg/L freeze less (p<0.05). C: All concentrations of fraction 1 recovered faster in the light challenge compared to the control group (2.5 mg/L p<0.01, 5.0 mg/L, 7.5 mg/L, 10 mg/L p<0.001). 2.5 mg/L, 7.5 mg/L and 10 mg/L were also found to freeze less in the light challenge (p<0.05, p<0.01, p<0.001, respectively). D: Concentrations 2.5 mg/L (p<0.001) 5.0 mg/L (p<0.001) and 7.5 mg/L (p<0.05) of fraction 2 recover faster in the light, all concentrations of fraction 2 freeze less in the light. Data (Mean±95% confidence interval), p>0.05; by linear mixed model. Replicates; DMSO n=70, Ethanol n=54, Fluoxetine n=53, Caffeine n=38, NaCl n=38, test product; 10 mg/L n=37, 12.5 mg/L n=36, 15 mg/L n=36. Fraction 1; 2.5 mg/L n=35, 5.0 mg/L n=36, 7.5 mg/L n=36, 10 mg/L n=36. Fraction 2; 2.5 mg/L n=35, 5.0 mg/L n=36, 7.5 mg/L n=36, 10 mg/L n=35. From top to bottom, relative to the starting point of each line at the left-hand y axis: A) Ethanol, Caffeine, NaCl, DMSO and Fluoxetine; B) 10 mg/L, 15 mg/L, 12.5 mg/L and DMSO; C) 10 mg/L, 7.5 mg/L, 2.5 mg/L, 5 mg/L, DMSO; D) 10 mg/L, 7.5 mg/L, 5 mg/L, DMSO, 2.5 mg/L.

FIG. 58—Comparing different compounds to DMSO in the dark period of the forced light/dark transition. Mean distance moved (mm) was measured every 10 s during the dark period, lasting 15 min. A) Shows mean distance moved (mm) over time of Ethanol, Fluoxetine, Caffeine and NaCl compared to DMSO over a period of 15 min in the dark. B) Shows mean distance moved (mm) over time of increasing dosages of test product compared to DMSO over a period of 15 min in the dark. C) Shows mean distance moved (mm) over time of increasing dosages of Fraction 1 compared to DMSO over a period of 15 min in the dark. D) Shows mean distance moved (mm) over time of increasing dosages of Fraction 2 compared to DMSO over a period of 15 min in the dark. A: All controls recover differently from DMSO (p<0.001). ethanol recovers faster and moves more (p<0.001). Fluoxetine and caffeine recover slower and move less than DMSO (p<0.001). NaCl recovers less (p<0.001). B: 10 mg/L test product recover faster (p<0.001), 15 mg/L test product recovers slower (p<0.01). 12.5 mg/L and 15 mg/L test product move less (p<0.001). C: 7.5 mg/L and 10 mg/L Fraction 1 recover faster (p<0.001). 2.5 mg/L (p<0.01), 7.5 mg/L (p<0.001) and 10 mg/L (p<0.001) move more in the beginning of the dark period. D: All concentrations of Fraction 2 recover faster than DMSO in the dark (p<0.001). All concentrations of Fraction 2 also moved more in the beginning of the dark period (2.5 mg/L p<0.01), 5.0 mg/L, 7.5 mg/L, 10 mg/L p<0.001). Data (Mean±95% confidence interval), p>0.05; by linear mixed model. Replicates; DMSO n=70, Ethanol n=54, Fluoxetine n=53, Caffeine n=38, NaCl n=38, test product; 10 mg/L n=37, 12.5 mg/L n=36, 15 mg/L n=36. Fraction 1; 2.5 mg/L n=35, 5.0 mg/L n=36, 7.5 mg/L n=36, 10 mg/L n=36. Fraction 2; 2.5 mg/L n=35, 5.0 mg/L n=36, 7.5 mg/L n=36, 10 mg/L n=35. From top to bottom, relative to the starting point of the line at the left-hand y axis: A) Ethanol, DMSO, NaCl, Fluoxetine and Caffeine; B) 10 mg/L, DMSO, 15 mg/L, 12.5 mg/L; C) 10 mg/L, 7.5 mg/L, 2.5 mg/L, 5 mg/L, DMSO; D) 7.5 mg/L, 10 mg/L, 5 mg/L, 2.5 mg/L, DMSO.

FIG. 59 shows UV chromatograms of Fractions 1 to 3.

FIG. 60 shows a UV chromatogram of Fraction 2.

FIG. 61 shows UV chromatograms of Fractions 1 to 3.

FIG. 62 shows MS2 chromatograms of Fractions 1 to 3. Unidentified compound.

FIG. 63 shows a UV spectrum of Fraction 2. Unidentified compound.

FIG. 64 shows MS chromatograms of Fractions 1 to 3. Unidentified compound.

FIG. 65 shows chromatograms of Fractions 1 to 3. Compound identified as cannflavin A.

FIG. 66 shows the results of a study on the anxiolytic effect of CBD in tank diving experiments. Panel A shows frequency of tank top half visits; panel B shows distance from tank bottom; and panel C shows proportion of time spent in bottom third of tank.

FIG. 67 shows the results of a study on the anxiolytic effect of CBD plus terpenes in tank diving experiments. Panel A shows frequency of tank top half visits; panel B shows distance from tank bottom; and panel C shows proportion of time spent in bottom third of tank.

FIG. 68 shows the results of a study on the anxiolytic effect of CBD plus CBG in tank diving experiments. Panel A shows frequency of tank top half visits; panel B shows distance from tank bottom; and panel C shows proportion of time spent in bottom third of tank.

### DETAILED DESCRIPTION

In general terms, the present invention relates to a composition comprising a cannabinoid, a terpene and optionally further components such as a carrier oil. Specific embodiments of the components of the composition will now be described.

### Cannabinoid

As disclosed herein, the cannabinoid may be selected from cannabidiol (CBD) and cannabigerol (CBG) and a mixture thereof. However, in the first aspect of the invention, the cannabinoid comprises cannabidiol (CBD). In some embodiments, the composition comprises CBD and at least one further cannabinoid.

In some embodiments, the cannabinoid is provided as an extract or extracts from a Cannabis plant, preferably of the species Cannabis sativa or Cannabis indica, whereas in other embodiments the cannabinoid is provided as a synthetic compound or a mixture of synthetic compounds, or as a chemically modified compound or mixture of chemically modified compounds. In other embodiments, the cannabinoid is provided as an extract from species of plant other than Cannabis plants, such as Echinacea purpurea, Echinacea angustifolia, Acemila oleracea, Helichrysum umbraculigerum, and Radula marginata.

In some embodiments, the cannabinoid is extracted from the Cannabis plant and the extract is further refined using a further stage of processing (for example, crystallisation technologies) which results in a crystallised, isolated cannabinoid, which is also referred to as an isolate or co-crystallised isolate. In some embodiments, the cannabinoid isolate has enhanced solubility and performance characteristics compared with the cannabinoid extract.

Cannabidiol (CBD), shown in formula (i) below, is a cannabinoid with the chemical formula C₂₁H₃₀O₂ and a molecular weight of 314.469 g/mol. The term “cannabidiol” also covers variants of CBD with different lengths of the alkyl chain, which vary in length from 1-5 carbons (and therefore the molecular weight of CBD varies from 266.426 g/mol to 314.469 g/mol). Depending on growing conditions it can constitute up to 40% of the extracts of the Cannabis sativa plant. CBD is not psychoactive or hallucinogenic.

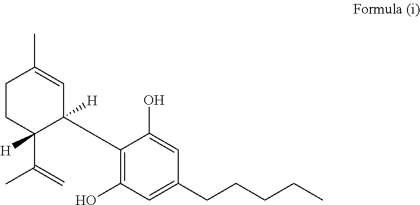 [SMILES: C=C(C)[C@@H]1CCC(C)=C[C@H]1c1c(O)cc(CCCCC)cc1O]

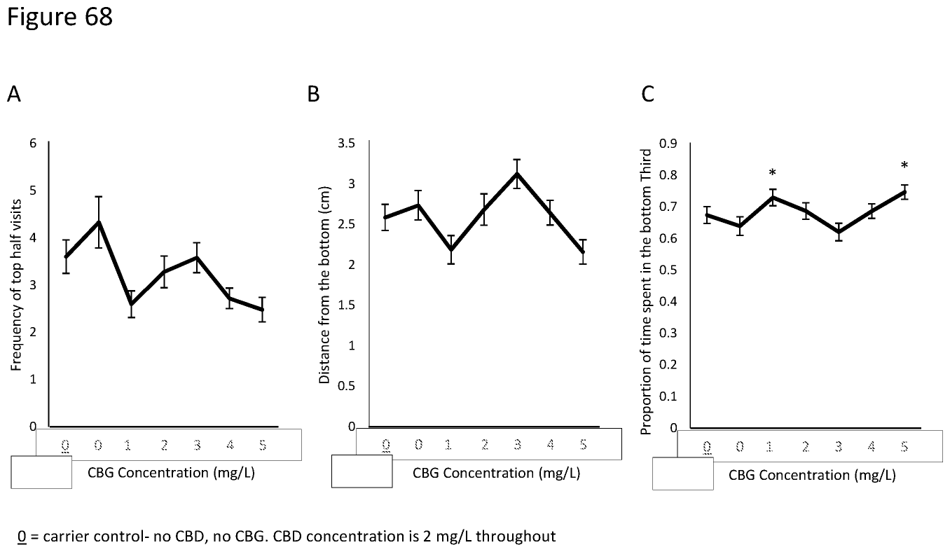

Cannabigerol (CBG), shown in formula (ii) below, is a cannabinoid with the chemical formula C₂₁H₃₂O₂ and a molecular weight of 316.485 g/mol. The term “cannabigerol” also covers variants of CBG with different lengths of the alkyl chain. Like CBD, CBG is not psychoactive or hallucinogenic. However, CBG is more expensive per kg than CBD and, in particular, CBG costs more than three times as much as CBD per kg. Therefore, for example, if one were to include an equal CBG:CBD ratio in the composition, the inclusion of CBG in the composition would more than double the total cannabinoid ingredient costs. Furthermore, it has now been surprisingly found that CBG does not have an anxiolytic effect, and may even have an undesirable anxiogenic effect, as demonstrated in Example 8. For example, FIG. 68 shows that the greater the concentration of CBG administered to the fish, the greater the amount of time the fish spent at the bottom of the tank, and the fewer the frequency of trips made to the top of the tank. The vertical position of the fish in the tank indicates an individual's anxiety, such that an increase in time the fish spends at the top of the tank indicates an increase in anxiety. Accordingly, it is preferred that the percentage w/w of CBG in the composition is kept to a minimum, in order to minimise costs and to avoid an anxiogenic effect. In some embodiments, the composition in total comprises <0.1% w/w CBG, preferably ≤0.05% w/w, ≤0.04% w/w, ≤0.03 w/w, ≤0.02% w/w or ≤ 0.01% w/w CBG, and more preferably ≤0.005% w/w, ≤0.004% w/w, ≤0.003% w/w, ≤0.002% w/w or ≤0.001% w/w CBG. In some embodiments, the composition comprises ≤0.0001% w/w CBG.

In some embodiments, the composition does not comprise CBG. In particular, this means that, in these embodiments, the composition comprises undetectable amounts of CBG, and preferably no CBG at all (i.e. 0% w/w).

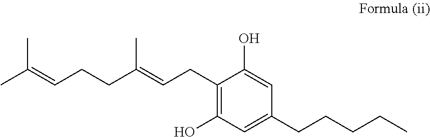 [SMILES: CCCCCc1cc(O)c(C/C=C(\C)CCC=C(C)C)c(O)c1]

In some embodiments of the present invention, the composition comprises CBD and CBG.

In some embodiments, the composition comprises ≤0.001% w/w of the cannabinoid 9-tetrahydrocannabinol (THC). In some embodiments, the composition comprises ≤0.0009% w/w, ≤0.0008% w/w, ≤0.0007% w/w, ≤0.0006% w/w, ≤0.0005% w/w, ≤0.0004% w/w, ≤0.0003% w/w, ≤0.0002% w/w, ≤0.0001% w/w, ≤0.00005% w/w or ≤0.00001% w/w THC. Any of these maximum amounts of THC may be used with any of the maximum amounts of CBG disclosed above. For example, the composition may comprise ≤0.0009% w/w THC and ≤0.05% w/w, ≤0.04% w/w, ≤0.03 w/w, ≤0.02% w/w, ≤0.01%, ≤0.005% w/w, ≤0.004% w/w, ≤0.003% w/w, ≤0.002% w/w or ≤ 0.001% w/w CBG; or the composition may comprise ≤0.00005% w/w THC and ≤0.05% w/w, ≤0.04% w/w, ≤0.03 w/w, ≤0.02% w/w, ≤0.01%, ≤0.005% w/w, ≤0.004% w/w, ≤0.003% w/w, ≤0.002% w/w or ≤0.001% w/w CBG; and so on.

In some embodiments, the composition does not comprise THC. In particular, this means that, in these embodiments, the composition comprises undetectable amounts of THC, and preferably no THC at all (i.e. 0% w/w).

In some embodiments, the composition comprises ≤0.0001% w/w of the cannabinoids cannabinol (CBN), 9-tetrahydrocannabinol (THC), and/or 9-tetrahydrocannabinolic acid (THC-A). In some embodiments, the composition comprises ≤0.0001% w/w of the cannabinoids cannabichromene (CBC) and/or cannabidiolic acid (CBD-A).

In some embodiments, the composition comprises neither THC nor CBG. In other words, each of THC and CBG is absent from the composition entirely (i.e. 0% w/w THC and 0% w/w CBG) or are present in undetectable amounts.

Tetrahydrocannabinol (THC; C₂₁H₃₀O₂), also known as delta-9-Tetrahydrocannabinol or (−)-trans-Δ⁹-Tetrahydrocannabinol, Cannabinol (CBN; C₂₁H₂₆O₂) and tetrahydrocannabinolic acid (THC-A; C₂₂H₂₀O₄) are psychoactive cannabinoids found in Cannabis plants, including the Cannabis sativa and Cannabis indica plants. These cannabinoids, and mixtures thereof, can also have other undesirable side effects. Thus, it is preferred to include no more than very low concentrations of each of THC, CBN and THC-A in the composition of the invention, and more preferably for each of CBN, THC and THC-A to be absent from the compositions of the invention.

In some embodiments, CBD is the only cannabinoid in the composition, i.e. the cannabinoid consists of CBD.

### Terpenes

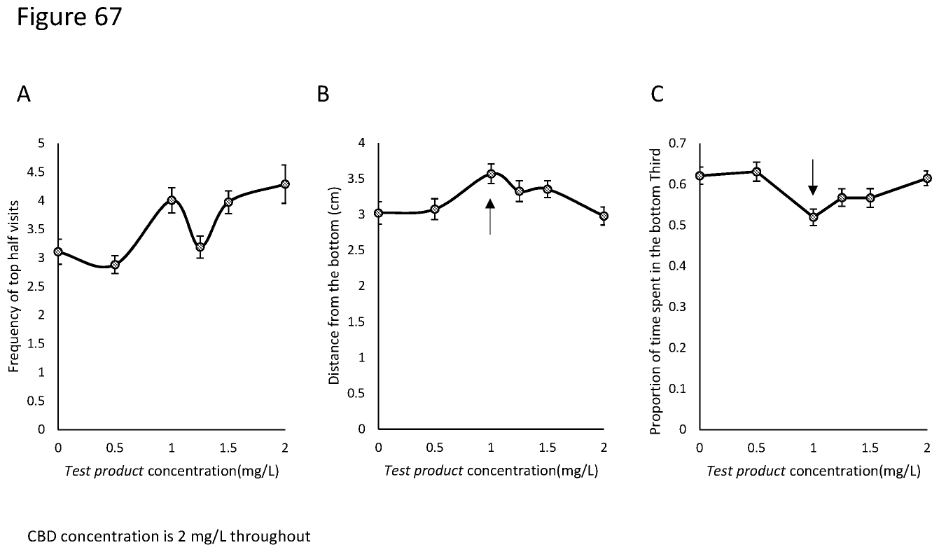

In the present invention, the composition comprises at least one terpene. In particular, it has been surprisingly found that the inclusion of a least one terpene in a composition comprising a cannabinoid increases the anxiolytic effect of the composition. For example, FIG. 67 shows that the anxiolytic effect of the compositions tested was increased when the composition included at least one terpene.

In some embodiments, the at least one terpene may comprise of two or more terpenes. For example, the composition may comprise 3, 4, 5, 6 or 7, terpenes. In some embodiments, the composition may contain only (i.e. the terpenes in the composition consist of) 1, 2, 3, 4, 5, 6 or 7 terpenes.

In some embodiments, the at least one terpene comprises alpha-humulene, terpinolene, beta-caryophyllene, alpha-pinene, beta pinene, beta myrcene (also known as myrcene), trans beta farnesene, caryophyllene oxide, trans alpha bergamotene, limonene, eudesmadiene, an ocimene isomer, a sesquiterpene, beta phellandrene, beta selinene, alpha selinene, 3-carene (also known as delta 3 carene), linalool, nerolidol, phytol, Inalyl acetate, borneol and/or cerolidol.

In some embodiments, the at least one terpene is terpinolene, beta myrcene and/or beta pinene, and the composition further comprises one or more terpenes selected from: alpha-humulene, beta-caryophyllene, alpha-pinene, trans beta farnesene, caryophyllene oxide, trans alpha bergamotene, limonene, eudesmadiene, an ocimene isomer, a sesquiterpene, beta phellandrene, beta selinene, alpha selinene, 3-carene (also known as delta 3 carene), linalool, nerolidol, phytol, Inalyl acetate, borneol and/or cerolidol. In some embodiments, the at least one terpene is terpinolene, beta myrcene and/or beta pinene, and the composition further comprises one or more terpenes selected from: alpha-humulene, beta-caryophyllene, an ocimene isomer (preferably trans beta ocimene) and alpha pinene.

In some embodiments, the at least one terpene comprises terpinolene and beta pinene, and at least one further terpene selected from alpha-humulene, beta-caryophyllene, alpha-pinene, beta-myrcene (also known as myrcene), trans beta farnesene, caryophyllene oxide, trans alpha bergamotene, limonene, eudesmadiene, an ocimene isomer, a sesquiterpene, beta phellandrene, beta selinene, alpha selinene, 3-carene (also known as delta 3 carene), linalool, nerolidol, phytol, Inalyl acetate, borneol and/or cerolidol.

In some embodiments, the at least one terpene comprises terpinolene and beta myrcene, and at least one further terpene selected from beta pinene, alpha-humulene, beta-caryophyllene, alpha-pinene, trans beta farnesene, caryophyllene oxide, trans alpha bergamotene, limonene, eudesmadiene, an ocimene isomer, a sesquiterpene, beta phellandrene, beta selinene, alpha selinene, 3-carene (also known as delta 3 carene), linalool, nerolidol, phytol, Inalyl acetate, borneol and cerolidol.

In some embodiments, the at least one terpene comprises or consists of alpha-humulene, terpinolene, beta-caryophyllene, alpha-pinene, beta-pinene, beta-myrcene (also known as myrcene) and an ocimene isomer (preferably trans beta ocimene).

In embodiments where the at least one terpene comprises beta myrcene, beta myrcene may be present in the greatest amount amongst all the terpenes present in the composition (in other words, beta myrcene is the dominant terpene). Thus, in some embodiments, the at least one terpene comprises beta myrcene, and one or both of terpinolene and beta pinene, wherein beta myrcene is the terpene present in the greatest amount in the composition.

In other embodiments, the terpene present in the greatest amount in the composition is one of alpha-humulene, terpinolene, beta-caryophyllene, alpha-pinene, beta pinene, trans beta farnesene, caryophyllene oxide, trans alpha bergamotene, limonene, eudesmadiene, an ocimene isomer, beta phellandrene, beta selinene, alpha selinene, 3-carene, linalool, nerolidol, phytol, Inalyl acetate, borneol or cerolidol, preferably terpinolene.

In some embodiments of the present invention, the composition comprises or consists of beta-caryophyllene, alpha-humulene, alpha-pinene, beta pinene, terpinolene, beta myrcene and an ocimene isomer. In some embodiments, the ocimene isomer is trans beta ocimene, and/or beta myrcene is the terpene present in the greatest amount in the composition.

In some embodiments, the at least one terpene is provided as an extract from a plant or insect. In some embodiments the at least one terpene is provided as an extract or extracts from a Cannabis plant, preferably of the species Cannabis sativa or Cannabis indica, whereas in other embodiments the at least one terpene is provided as a synthetic compound or a mixture of synthetic compounds, or as a chemically modified compound or mixture of chemically modified compounds.

The composition may consist essentially of a cannabinoid selected from CBD and CBG and a terpene selected from: beta-caryophyllene, alpha-humulene, alpha-pinene, beta pinene, terpinolene, beta myrcene, an ocimene isomer and mixtures thereof. In some instances, the terpene:cannabinoid ratio is 1:3 to 1:60 w/w, preferably 1:3 to 1:40 w/w, 1:3 to 1:35 w/w, 1:3 to 1:20 w/w, 1:5 to 1:20 w/w, 1:5 to 1:9 w/w, 1:5 to 1:7 w/w, 1:7 to 1:12 w/w, 1:9 to 1:20 w/w, or 1:15 to 1:20 w/w.

In some embodiments, the composition consists essentially of CBD and one or more terpenes selected from: beta-caryophyllene, alpha-humulene, alpha-pinene, beta pinene, terpinolene, beta myrcene and an ocimene isomer, wherein the ocimene isomer is preferably trans beta ocimene. In preferred variants of this embodiment, the terpene:cannabinoid ratio is 1:3 to 1:60 w/w, preferably 1:3 to 1:40 w/w, 1:3 to 1:35 w/w, 1:3 to 1:20 w/w, 1:5 to 1:20 w/w, 1:5 to 1:9 w/w, 1:5 to 1:7 w/w, 1:7 to 1:12 w/w, 1:9 to 1:20 w/w, or 1:15 to 1:20 w/w.

In some embodiments, the composition comprises beta-caryophyllene, alpha-humulene, alpha-pinene, beta-pinene, terpinolene, beta-myrcene, an ocimene isomer and a least one other sesquiterpene.

In all embodiments which comprise an ocimenene isomer, the ocimene isomer is preferably trans beta ocimene.

In some embodiments, the at least one terpene in the composition consists of beta myrcene, alpha-humulene, beta-caryophyllene, alpha-pinene, beta-pinene, terpinolene, and an ocimene isomer, and wherein the terpene present in the greatest amount is beta-myrcene.

In some embodiments, the composition comprises beta myrcene at 0.05% w/w to 0.2% w/w, 0.05% w/w to 0.19% w/w, 0.05% w/w to 0.18% w/w, 0.06% w/w to 0.18% w/w, 0.07% w/w to 0.17% w/w, 0.08% w/w to 0.16% w/w, 0.08% w/w to 0.15% w/w, 0.85% w/w to 0.141% w/w, 0.086% w/w to 0.141% w/w or 0.87% w/w to 0.142% w/w. In some embodiments, the composition comprises beta myrcene at 0.080% w/w to 0.090% w/w, 0.081% w/w to 0.090% w/w, 0.82% w/w to 0.090% w/w, 0.83% w/w to 0.090% w/w, 0.084% w/w to 0.090% w/w, 0.085% w/w to 0.090% w/w, 0.085% w/w to 0.089% w/w, or 0.086% w/w to 0.088% w/w. In some embodiments, the composition comprises beta myrcene at 0.100% w/w to 0.110% w/w, 0.101% w/w to 0.0110% w/w, 0.102 to 0.110% w/w, 0.103% w/w to 0.110% w/w, 0.104% w/w to 0.110% w/w, 0.105% w/w to 0.110% w/w, 0.105% w/w to 0.109% w/w, 0.106% w/w to 0.109% w/w or 0.107% w/w to 0.109% w/w. In some embodiments, the composition comprises beta myrcene at 0.140% w/w to 0.150% w/w, 0.140% w/w to 0.149% w/w, 0.140% w/w to 0.148% w/w, 0.140% w/w to 0.147% w/w, 0.140% w/w to 0.146% w/w, 0.140% w/w to 0.145% w/w, 0.140% w/w to 0.144% w/w, 0.140% w/w to 0.143% w/w, 0.141% w/w to 0.143% w/w. In some embodiments, the composition comprises beta myrcene at 0.087% w/w, 0.108% w/w or 0.142% w/w.

In some embodiments, the composition comprises terpinolene at 0.01% w/w to 0.2% w/w, 0.01% w/w to 0.19% w/w, 0.01% w/w to 0.18% w/w, 0.02% w/w, to 0.18% w/w, 0.03% w/w to 0.18% w/w, 0.04% w/w to 0.18% w/w, 0.04% w/w to 0.17% w/w, 0.04% w/w to 0.16% w/w, 0.04% w/w to 0.15% w/w, 0.04% w/w to 0.15% w/w, 0.04% w/w to 0.14% w/w, 0.04% w/w to 0.13% w/w, 0.05% w/w to 0.13% w/w, 0.05% w/w to 0.12% w/w, 0.05% w/w to 0.11% w/w, 0.06% w/w to 0.11% w/w, 0.06% w/w to 0.1% w/w or 0.06% w/w to 0.098% w/w. In some embodiments, the composition comprises terpinolene at 0.055% w/w to 0.065% w/w, 0.056% w/w to 0.065% w/w, 0.057% w/w to 0.065% w/w, 0.057% w/w to 0.064% w/w, 0.057% w/w to 0.063% w/w, 0.058% w/w to 0.063% w/w, 0.058% w/w to 0.062% w/w or 0.059% w/w to 0.061% w/w. In some embodiments, composition comprises terpinolene at 0.068% w/w to 0.08% w/w, 0.068% w/w to 0.079% w/w, 0.068% w/w to 0.078% w/w, 0.068% w/w to 0.077% w/w, 0.069% w/w to 0.077% w/w, 0.070% w/w to 0.077% w/w, 0.071% w/w to 0.077% w/w, 0.072% w/w to 0.077% w/w, 0.073% w/w to 0.077% w/w, 0.073% w/w to 0.076% w/w or 0.073% w/w to 0.075% w/w. In some embodiments, the composition comprises terpinolene at 0.090% w/w to 0.15% w/w, 0.091% w/w to 0.15% w/w, 0.092% w/w to 0.15% w/w, 0.093% w/w to 0.15% w/w, 0.094% w/w to 0.15% w/w, 0.095% w/w to 0.15% w/w, 0.095% w/w to 0.14% w/w, 0.095% w/w to 0.13% w/w, 0.095% w/w to 0.12% w/w, 0.095% w/w to 0.11% w/w, 0.096% w/w to 0.11% w/w, 0.097% w/w to 0.11% w/w, 0.097% w/w to 0.10% w/w, 0.097% w/w to 0.099% w/w or 0.098% w/w to 0.099% w/w. In some embodiments, the composition comprise terpinolene at 0.06% w/w, 0.074% w/w or 0.098% w/w.

In some embodiments, the composition comprises beta pinene at 0.01% w/w to 0.05% w/w, 0.01% w/w to 0.045% w/w, 0.015% w/w to 0.045% w/w, 0.015% w/w to 0.04% w/w, 0.019% w/w to 0.04% w/w, 0.021% w/w to 0.04% w/w, 0.021% w/w to 0.039% w/w, 0.022% w/w to 0.039% w/w, 0.023% w/w to 0.039% w/w or 0.023% w/w to 0.038% w/w. In some embodiments, the composition comprises beta pinene at 0.015% w/w to 0.03% w/w, 0.016% w/w 0.03% w/w, 0.017% w/w to 0.03% w/w, 0.018% w/w to 0.03% w/w, 0.018% w/w to 0.029% w/w, 0.018% w/w to 0.028% w/w, 0.019% w/w to 0.028% w/w, 0.019% w/w to 0.028% w/w, 0.020% w/w to 0.028% w/w, 0.020% w/w to 0.027% w/w, 0.021% w/w to 0.027% w/w, 0.021% w/w to 0.026% w/w, 0.022% w/w to 0.026% w/w, 0.022% w/w to 0.025% w/w or 0.023% w/w to 0.026% w/w. In some embodiments, the composition comprises beta-pinene at 0.020% w/w to 0.35% w/w, 0.022% w/w to 0.035% w/w, 0.022% w/w to 0.034% w/w, 0.023% w/w to 0.034% w/w, 0.024% w/w to 0.034% w/w, 0.024% w/w to 0.033% w/w, 0.025% w/w to 0.033% w/w, 0.025% w/w to 0.032% w/w, 0.026% w/w to 0.032% w/w, 0.026% w/w to 0.031% w/w, 0.027% w/w to 0.031% w/w, 0.28% w/w to 0.031% w/w or 0.028% w/w to 0.030% w/w. In some embodiments, the composition comprises beta pinene at 0.03% w/w to 0.045% w/w, 0.03% w/w to 0.044% w/w, 00.031% w/w to 0.044 w/w, 0.032% w/w to 0.044% w/w, 0.033% w/w to 0.044% w/w, 0.034% w/w to 0.044% w/w, 0.035% w/w to 0.044% w/w, 0.035% w/w to 0.042% w/w, 0.36% w/w to 0.042% w/w, 0.036% w/w to 0.041% w/w, 0.037% w/w to 0.041% w/w, 0.037% w/w t 0.040% w/w, 0.038% w/w to 0.040% w/w or 0.038% w/w to 0.040% w/w. In some embodiments, the composition comprises beta pinene at 0.024% w/w, 0.030% w/w or 0.039% w/w.

In some embodiments, the at least one terpene comprises beta myrcene at 0.142% w/w, terpinolene at 0.098% w/w and beta pinene at 0.039% w/w. In some embodiments, the composition comprises beta myrcene at 0.108% w/w, terpinolene at 0.074% w/w and beta pinene at 0.030% w/w. In some embodiments, the composition comprises beta myrcene at 0.087% w/w, terpinolene at 0.06% w/w and beta pinene at 0.024% w/w.

In some embodiments, the at least one terpene comprises or consists of beta myrcene at 0.142% w/w, beta-caryophyllene at 0.118% w/w, terpinolene at 0.098% w/w, alpha pinene at 0.081% w/w, trans beta ocimene at 0.068% w/w, alpha-humulene at 0.051% w/w, and beta-pinene at 0.039% w/w. Embodiments in which the at least one terpene consists of beta myrcene at 0.142% w/w, beta-caryophyllene at 0.118% w/w, terpinolene at 0.098% w/w, alpha pinene at 0.081% w/w, trans beta ocimene at 0.068% w/w, alpha-humulene at 0.051% w/w, and beta-pinene at 0.039% w/w are referred to herein as Formula 1.

In some embodiments, the at least one terpene comprises or consists of beta myrcene at 0.108% w/w, beta-caryophyllene at 0.089% w/w, terpinolene at 0.074% w/w, alpha pinene at 0.061% w/w, trans beta ocimene at 0.052% w/w, alpha-humulene at 0.038% w/w, and beta-pinene at 0.030% w/w. Embodiments in which the at least one terpene consists of beta myrcene at 0.108% w/w, beta-caryophyllene at 0.089% w/w, terpinolene at 0.074% w/w, alpha pinene at 0.061% w/w, trans beta ocimene at 0.052% w/w, alpha-humulene at 0.038% w/w, and beta-pinene at 0.030% w/w are referred to herein as Formula 2.

In some embodiments, the composition comprises or consists of beta myrcene at 0.087% w/w, beta-caryophyllene at 0.072% w/w, terpinolene at 0.060% w/w, alpha pinene at 0.050% w/w, trans beta ocimene at 0.042% w/w, alpha-humulene at 0.031% w/w, and beta-pinene at 0.024% w/w. Embodiments in which the at least one terpene consists of beta-myrcene at 0.087% w/w, beta-caryophyllene at 0.072% w/w, terpinolene at 0.060% w/w, alpha pinene at 0.050% w/w, trans beta ocimene at 0.042% w/w, alpha-humulene at 0.031% w/w, and beta-pinene at 0.024% w/w are referred to herein as Formula 3.

Terpenes are a large and diverse group of organic compounds, in particular hydrocarbons, produced by a variety of plants, including Cannabis sativa and Cannabis indica. Terpenes are classified by the number of isoprene units that are present in the molecule; for example, monoterpenes consist of two isoprene units and have the molecular formula C₁₀H₁₆. Limonene, terpinolene and pinene, for which there are two structural isomers alpha-pinene and beta-pinene, are examples of monoterpenes. Sesquiterpenes consist of three isoprene units and have the formula C₁₅H₂₄. Humulene, which is also known as alpha-humulene, and beta-caryophyllene are examples of sesquiterpenes.

The structures of two terpenes, alpha-humulene (a sesquiterpene; formula (iii)) and terpinolene (a monoterpene; formula (iv)), are shown below.

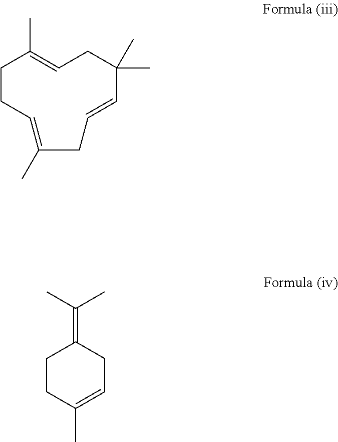

The profile of terpenes present in the composition contributes to the flavour and smell of the composition.

Terpenoids are modified terpenes that contain additional functional groups. In some embodiments of the present invention, the at least one terpene comprises at least one terpenoid.

The terpene(s) and cannabinoid(s) may be present in the composition in a ratio of 1:3 to 1:60 w/w, 1:3 to 1:55, 1:3 to 1:45, 1:3 to 1:40 w/w, 1:3 to 1:35 w/w, 1:3 to 1:20 w/w, 1:5 to 1:20 w/w, 1:5 to 1:9 w/w, 1:5 to 1:7 w/w, 1:7 to 1:12 w/w, 1:8 to 1:55, 1:8 to 1:45, 1:8 to 1:35, 1:8 to 1:30, 1:8 to 1:25, 1:8 to 1:20, 1:8 to 1:15, 1:9 to 1:20 w/w, or 1:15 to 1:20 w/w. In some embodiments, the terpene:cannabinoid ratio is 1:8 w/w, 1:11 w/w, 1:14 w/w, 1:17 w/w, 1:22 w/w, 1:27 w/w, 1:34 w/w, 1:44 w/w or 1:55 w/w.

In some embodiments, the terpene(s) and CBD are present in the composition in a ratio of 1:3 to 1:60 w/w, 1:3 to 1:55, 1:3 to 1:45, 1:3 to 1:40 w/w, 1:3 to 1:35 w/w, 1:3 to 1:20 w/w, 1:5 to 1:20 w/w, 1:5 to 1:9 w/w, 1:5 to 1:7 w/w, 1:7 to 1:12 w/w, 1:8 to 1:55, 1:8 to 1:45, 1:8 to 1:35, 1:8 to 1:30, 1:8 to 1:25, 1:8 to 1:20, 1:8 to 1:15, 1:9 to 1:20 w/w, or 1:15 to 1:20 w/w. In some embodiments, the terpene:CBD ratio is 1:8 w/w, 1:11 w/w, 1:14 w/w, 1:17 w/w, 1:22 w/w, 1:27 w/w, 1:34 w/w, 1:44 w/w or 1:55 w/w. In the embodiments of this paragraph, it is preferred that CBD is the only cannabinoid in the composition.

In some embodiments, the amount of CBD in the composition is between 1% w/w and 30% w/w, between 3% w/w and 30% w/w, between 3% w/w and 25% w/w, or between 3% w/w and 20% w/w. In some embodiments, the amount of CBD in the composition is 1% w/w, 2% w/w, 3% w/w, 4% w/w, 5% w/w, 10% w/w, 15% w/w, 20% w/w, 25% w/w or 30% w/w. In some embodiments, the amount of CBD in the composition is 3% w/w, 5% w/w, 10% w/w or 20% w/w, preferably 20% w/w. Any of these amounts of CBD may be used with any of the terpenes and terpene mixtures described above. For example, in some embodiments, the composition comprises CBD in an amount of between 1% w/w and 30% w/w, between 3% w/w and 30% w/w, between 3% w/w and 25% w/w, or between 3% w/w and 20% w/w, preferably at 3% w/w, 5% w/w, 10% w/w or 20% w/w, and the at least one terpene comprises terpinolene, beta myrcene and/or beta pinene. In some embodiments, the composition comprises CBD at 3% w/w, 5% w/w, 10% w/w or 20% w/w and the at least one terpene comprises terpinolene. In some embodiments, the composition comprises 3% w/w, 5% w/w, 10% w/w or 20% w/w and the at least one terpene comprises beta myrcene. In some embodiments, the composition comprises CBD at 3% w/w, 5% w/w, 10% w/w or 20% w/w and the at least one terpene comprises beta pinene. In some embodiments, the composition comprises CBD at 3% w/w, 5% w/w, 10% w/w or 20% w/w and the at least one terpene comprises terpinolene, beta pinene and beta myrcene. In some embodiments, the composition comprises CBD at 3% w/w, 5% w/w, 10% w/w or 20% w/w, preferably 20% w/w, and the at least one terpene comprises or consists of beta-caryophyllene, alpha-humulene, alpha-pinene, beta-pinene, terpinolene, beta-myrcene and an ocimene isomer.

In some embodiments, the terpenes in the composition are according to Formula 1 above, and CBD is present at 3% w/w.

In some embodiments, the terpenes in the composition are according to Formula 1 above, and the terpene:CBD ratio is 1:8 w/w, preferably wherein CBD is present at 5% w/w.

In some embodiments, the terpenes in the composition are according to Formula 1 above, and the terpene:CBD ratio is 1:17 w/w, preferably wherein CBD is present at 10% w/w.

In some embodiments, the terpenes in the composition are according to Formula 1 above, and the terpene:CBD ratio is 1:34 w/w, preferably wherein CBD is present at 20% w/w.

In some embodiments, the terpenes in the composition are according to Formula 2 above, and CBD is present at 3% w/w.

In some embodiments, the terpenes in the composition are according to Formula 2 above, and the terpene:CBD ratio is 1:11 w/w, preferably wherein CBD is present at 5% w/w.

In some embodiments, the terpenes in the composition are according to Formula 2 above, and the terpene:CBD ratio is 1:22 w/w, preferably wherein CBD is present at 10% w/w.

In some embodiments, the terpenes in the composition are according to Formula 2 above, and the terpene:CBD ratio is 1:44 w/w, preferably wherein CBD is present at 20% w/w.

In some embodiments, the terpenes in the composition are according to Formula 3 above, and CBD is present at 3% w/w.

In some embodiments, the terpenes in the composition are according to Formula 3 above, and the terpene:CBD ratio is 1:14 w/w, preferably wherein CBD is present at 5% w/w.

In some embodiments, the terpenes in the composition are according to Formula 3 above, and the terpene:CBD ratio is 1:27 w/w, preferably wherein CBD is present at 10% w/w.

In some embodiments, the terpenes in the composition are according to Formula 3 above, and the terpene:CBD ratio is 1:55 w/w, preferably wherein CBD is present at 20% w/w.

### Flavonoids

In embodiments of the present invention, the composition comprises one or more flavonoids. Flavonoids are produced by a variety of plants and have the general structure of a 15-carbon skeleton, which consists of two phenyl rings and a heterocyclic ring. This carbon structure is sometimes referred to as a C6-C3-C6 structure.

In some embodiments, the one or more flavonoids are provided as an extract from a plant. In some embodiments the one or more flavonoids are provided as an extract or extracts from a Cannabis plant, preferably of the species Cannabis sativa or Cannabis indica, whereas in other embodiments the one or more flavonoids are provided as a synthetic compound or a mixture of synthetic compounds, or as a chemically modified compound or mixture of chemically modified compounds.

In some embodiments, the one or more flavonoids is selected from: a flavone, a flavonol and a cannflavin. In some embodiments, the one or more flavonoids is cannflavin A, cannflavin B, cannflavin C, apigenin, luteolin, kaempferol, quercetin, chrysoeriol, isocannflavin B and myricetin. In some embodiments, the one or more flavonoid is a cannflavin, preferably cannflavin A, cannflavin B or cannflavin C. In embodiments where there is more than one flavonoid in the composition, it is preferred that at least one of the flavonoids is a cannflavin. In some embodiments in which there is more than one flavonoid, each flavonoid is a cannflavin.

### Carrier Oil

In some embodiments of the present invention, the composition comprises a carrier oil. The characteristics of the carrier oil are not limited except that the oil must not interact with the other components of the composition. Preferably, the oil is an oil which is defined as GRAS (generally recognised as safe for human consumption). In some embodiments of the present invention, the composition comprises a carrier oil is selected from: hemp seed oil, coconut oil, rape seed oil, medium-chain triglyceride (MCT) oil, olive oil, cranberry oil and vegetable oil, or a mixture of two or more thereof. In other embodiments, the carrier oil is a mixture of hemp seed oil and coconut oil. In other embodiments, the carrier oil is a mixture of rape seed oil and coconut oil.

As such, in some embodiments the carrier oil dilutes the concentration of the other components of the composition. However, it is to be appreciated that the ratio of the other components to the cannabinoid remains the same following such dilution.

The cannabinoid:oil ratio may be 1:2 to 1:3500 w/w in the composition. For example, the cannabinoid:oil ratio may be 1:2 to 1:2000 w/w, 1:3 to 1:500 w/w, 1:3 to 1:200 w/w, 1:3 to 1:5 w/w, 1:5 to 1:500 w/w, 1:20 to 1:100 w/w or 1:50 to 1:100 w/w. In some embodiments, the CBD:oil ratio is 1:2 to 1:3500 w/w in the composition. In some embodiments, the CBD:oil ratio is 1:2 to 1:2000 w/w, 1:3 to 1:500 w/w, 1:3 to 1:200 w/w, 1:3 to 1:5 w/w, 1:5 to 1:500 w/w, 1:20 to 1:100 w/w or 1:50 to 1:100 w/w

It is to be understood that, in some embodiments, the composition is provided without a carrier oil. In these embodiments, the composition is provided in a concentrated form, which may not be generally safe for human consumption due to the concentration of cannabinoid. In these embodiments, the concentrated form of the composition is provided to a business user or end user for mixing with carrier oil before use. Thus, in some embodiments, there is provided the composition in concentrated or isolate form and a suitable amount of carrier oil (in a receptacle, for example). The composition in concentrate or isolate form and the carrier oil, may be provided as a kit, in which the composition and the carrier oil are provided in separate vials or receptacles. In these embodiments, the end user combines the carrier oil and the concentrate or isolate to provide a final, dilute product in which the concentration of cannabinoid and other components are suitable for human consumption.

### Nature of the Formulation

In some embodiments of the present invention, the composition is produced as a concentrate, an isolate, a co-crystallised isolate, an oil, a liposomal formulation, a food bar, a savoury or sweet food such as a nutritional food bar or chocolate, a beverage, a pharmaceutical composition, or it may be nano-encapsulated, or emulsified or formed into an aerosol. In other embodiments of the present invention, the composition is produced as a tablet, capsule, cream, ointment, pessary or suppository. In other embodiments, the composition can constitute less than 10 wt % or less than 5 wt % of a food bar or beverage.

### Exemplary Compositions

Some exemplary compositions of the present invention are set out below:

Composition 1 (terpene:cannabinoid is 1:5 w/w and cannabinoid:oil is 1:18.8 w/w): 5% w/w CBD, 0.2% w/w alpha-pinene, 0.2% w/w beta-pinene, 0.2% w/w beta-caryophyllene, 0.2% w/w alpha-humulene, 0.2% w/w terpinolene (1% w/w terpenes in total) and 94% w/w carrier oil.

Composition 2 (terpene:cannabinoid is 1:20 w/w and cannabinoid:oil is 1:9 w/w): 10% w/w CBD, 0.12% w/w alpha-pinene, 0.2% w/w beta-pinene, 0.05% w/w beta-caryophyllene, 0.03% w/w alpha-humulene, 0.1% w/w terpinolene (0.5% w/w terpenes in total) and 89.5% w/w carrier oil.

Composition 3 (terpene:cannabinoid is 1:9 w/w): 90% w/w CBD, 2% w/w alpha-pinene, 2.6% w/w beta-pinene, 1.5% w/w beta-caryophyllene, 2% w/w alpha-humulene and 1.9% w/w terpinolene (10% w/w terpenes in total).

Composition 4 (terpene:cannabinoid is 1:15 w/w and cannabinoid:oil is 1:12.3 w/w): 7.5% w/w CBD, 0.5% w/w terpinolene and 92% w/w carrier oil.

Composition 5 (terpene:cannabinoid is 1:60 w/w and cannabinoid:oil is 1:2.3 w/w): 30% w/w CBD, 0.15% w/w alpha-pinene, 0.2% w/w beta-pinene, 0.1% w/w beta-caryophyllene, 0.15% w/w alpha-humulene, 0.15% w/w terpinolene (0.75% w/w terpenes in total) and 69.25% w/w carrier oil.

Composition 6 (terpene:cannabinoid is 1:16.7 w/w and cannabinoid:oil is 1:2.9 w/w): 25% CBD, 0.1% w/w alpha-pinene, 0.3% w/w beta-pinene, 0.5% w/w beta-caryophyllene, 0.1% w/w alpha-humulene, 0.2% w/w terpinolene, 0.2% w/w beta-myrcene, 0.1% w/w trans beta-ocimene and 73.5% w/w carrier oil.

Composition 7 (terpene:CBD is 1:8 w/w): CBD, beta myrcene, alpha-humulene, beta-caryophyllene, alpha-pinene, beta-pinene, terpinolene, and trans beta ocimene, specifically 5% w/w CBD, 0.142% w/w, beta-caryophyllene at 0.118% w/w, terpinolene at 0.098% w/w, alpha pinene at 0.081% w/w, trans beta ocimene at 0.068% w/w, alpha-humulene at 0.051% w/w, and beta-pinene at 0.039% w/w.

Composition 8 (terpene:CBD is 1:17 w/w): CBD, beta myrcene, alpha-humulene, beta-caryophyllene, alpha-pinene, beta-pinene, terpinolene and trans beta ocimene, specifically 10% w/w CBD, 0.142% w/w, beta-caryophyllene at 0.118% w/w, terpinolene at 0.098% w/w, alpha pinene at 0.081% w/w, trans beta ocimene at 0.068% w/w, alpha-humulene at 0.051% w/w, and beta-pinene at 0.039% w/w.

Composition 9 (terpene:CBD is 1:34 w/w): CBD, beta myrcene, alpha-humulene, beta-caryophyllene, alpha-pinene, beta-pinene, terpinolene and trans beta ocimene, specifically, 20% w/w CBD, 0.142% w/w, beta-caryophyllene at 0.118% w/w, terpinolene at 0.098% w/w, alpha pinene at 0.081% w/w, trans beta ocimene at 0.068% w/w, alpha-humulene at 0.051% w/w, and beta-pinene at 0.039% w/w.

Composition 10 (terpene:CBD is 1:11 w/w): CBD, beta myrcene, alpha-humulene, beta-caryophyllene, alpha-pinene, beta-pinene, terpinolene and trans beta ocimene, specifically 5% w/w CBD, beta-myrcene at 0.108% w/w, beta-caryophyllene at 0.089% w/w, terpinolene at 0.074% w/w, alpha pinene at 0.061% w/w, trans beta ocimene at 0.052% w/w, alpha-humulene at 0.038% w/w, and beta-pinene at 0.030% w/w.

Composition 11 (terpene:CBD is 1:22 w/w): CBD, beta myrcene, alpha-humulene, beta-caryophyllene, alpha-pinene, beta-pinene, terpinolene and trans beta ocimene, specifically. The compositions consists of 10% CBD, beta-myrcene at 0.108% w/w, beta-caryophyllene at 0.089% w/w, terpinolene at 0.074% w/w, alpha pinene at 0.061% w/w, trans beta ocimene at 0.052% w/w, alpha-humulene at 0.038% w/w, and beta-pinene at 0.030% w/w.

Composition 12 (terpene:CBD is 1:44 w/w): CBD, beta myrcene, alpha-humulene, beta-caryophyllene, alpha-pinene, beta-pinene, terpinolene and trans beta ocimene, specifically 20% w/w CBD, beta-myrcene at 0.108% w/w, beta-caryophyllene at 0.089% w/w, terpinolene at 0.074% w/w, alpha pinene at 0.061% w/w, trans beta ocimene at 0.052% w/w, alpha-humulene at 0.038% w/w, and beta-pinene at 0.030% w/w.

Composition 13 (terpene:CBD is 1:14 w/w): CBD, beta myrcene, alpha-humulene, beta-caryophyllene, alpha-pinene, beta-pinene, terpinolene and trans beta ocimene, specifically 5% w/w CBD, beta-myrcene at 0.087% w/w, beta-caryophyllene at 0.072% w/w, terpinolene at 0.060% w/w, alpha pinene at 0.050% w/w, trans beta ocimene at 0.042% w/w, alpha-humulene at 0.031% w/w, and beta-pinene at 0.024% w/w.

Composition 14 (terpene:CBD is 1:27 w/w): CBD, beta myrcene, alpha-humulene, beta-caryophyllene, alpha-pinene, beta-pinene, terpinolene and trans beta ocimene, specifically, 10% w/w CBD, beta-myrcene at 0.087% w/w, beta-caryophyllene at 0.072% w/w, terpinolene at 0.060% w/w, alpha pinene at 0.050% w/w, trans beta ocimene at 0.042% w/w, alpha-humulene at 0.031% w/w, and beta-pinene at 0.024% w/w.

Composition 15 (terpene:CBD is 1:55 w/w): CBD, beta myrcene, alpha-humulene, beta-caryophyllene, alpha-pinene, beta-pinene, terpinolene and trans beta ocimene, specifically 20% w/w CBD, beta-myrcene at 0.087% w/w, beta-caryophyllene at 0.072% w/w, terpinolene at 0.060% w/w, alpha pinene at 0.050% w/w, trans beta ocimene at 0.042% w/w, alpha-humulene at 0.031% w/w, and beta-pinene at 0.024% w/w.

### Use

The composition of the present invention is used in medicine (i.e. as a medicament) in a clinical setting, preferably, for the treatment or prevention of anxiety or a disorder associated with anxiety. In such a clinical setting, the composition of the present invention is preferably provided in a pharmaceutical-style (i.e. pharmaceutically-acceptable) formulation, more preferably the formulation comprises a pharmaceutical excipient and/or diluent.

In one aspect of the invention, there is provided a pharmaceutical formulation comprising the composition of the invention and a pharmaceutically-acceptable carrier, diluent or excipient.

In some embodiments, the composition or pharmaceutical formulation of the present invention is used for the treatment or prophylaxis of a human individual or animal, preferably the treatment or prevention of anxiety or a disorder associated with anxiety.

In some embodiments, the composition is used in the preparation of a pet food or pet food additive. In some embodiments, the pet food or pet food additive is for use in the treatment

However, it is also to be understood that in alternative embodiments, the composition or pharmaceutical formulation of the present invention is used in order to alleviate anxiety of a sub-clinical nature. In such alternative embodiments, the composition or pharmaceutical formulation of the present invention is preferably provided in a food or beverage product, preferably in a chewing gum, lozenge or tincture, or a tablet or capsule which is dissolvable in a beverage.

In some embodiments, the composition or pharmaceutical formulation is used to prevent anxiety or a disorder associated with anxiety. For example, in some embodiments, the composition is administered or consumed in advance of a period when anxiety or a disorder associated with anxiety is otherwise expected.

### Administration

In some embodiments, the composition or pharmaceutical formulation of the present invention is administered by ingestion (for example from drinking, eating or consuming capsules or tablets), buccal or nasal absorption, oral absorption (for example from tinctures, lozenges or chewing gum), inhalation (for example, from vaping, a dry powder inhaler or a nebuliser), systemic injection, or transdermal or topical absorption (for example from patches, creams, ointments, pessaries and suppositories). In some embodiments of the present invention, the composition or pharmaceutically-acceptable formulation is administered through a dropper or spray. In other embodiments of the present invention, the composition or pharmaceutically-acceptable formulation is administered intravenously.

In some embodiments, the daily dosage is varied to suit the individual human or animal (for example to take into account the medical condition being treated). In some embodiments, the frequency of use or administration determines the daily dosage of the composition of the present invention. In some embodiments, the composition pharmaceutical formulation of the present invention is delivered as a single dose per day. In other embodiments, the composition pharmaceutical formulation of the present invention is delivered in multiple, smaller doses per day up to a total daily dosage. In certain embodiments, the composition pharmaceutical formulation of the present invention is delivered by microdosing or by way of a bolus.

As disclosed herein, a composition comprising 1-30% w/w CBD or CBG, may be administered through a dropper, delivers 0.8-24 mg CBD or CBG, or a mixture thereof, per drop. As disclosed herein, an individual human or animal requiring a daily dose of 12 mg CBD or CBG may be administered over the course of 24 hours with one drop of a composition comprising 15% w/w CBD or CBG, or a mixture thereof, per day. Alternatively, an individual human or animal requiring a daily dose of 12 mg CBD or CBG may be administered over the course of 24 hours with two drops of a composition comprising 7.5% w/w CBD or CBG, or a mixture thereof.

In some embodiments of the present invention, a composition comprising 1-30% CBD, administered through a dropper, delivers 0.8-24 mg CBD per drop. In some embodiments, an individual human or animal requiring a daily dose of 12 mg CBD is administered over the course of 24 hours with one drop of a composition comprising 15% w/w CBD per day. In alternative embodiments, an individual human or animal requiring a daily dose of 12 mg CBD is administered over the course of 24 hours with two drops of a composition comprising 7.5% w/w CBD.

Disclosed herein is a composition comprising 1-30% w/w CBD or CBG, administered through a spray, delivers 1.4-42 mg CBD or CBG per spray. As disclosed herein, an individual human or animal requiring a daily dose of 30 mg CBD or CBG may be administered over the course of 24 hours with one spray of a composition comprising 21.4% w/w CBD or CBG, or a mixture thereof, per day. Alternatively, an individual human or animal requiring a daily dose of 30 mg CBD or CBG may be administered over the course of 24 hours with four sprays of a composition comprising 5.35% w/w CBD or CBG, or a mixture thereof.

In some embodiments, a composition comprising 1-30% w/w CBD, administered through a spray, delivers 1.4-42 mg CBD per spray. In some embodiments, an individual human or animal requiring a daily dose of 30 mg CBD is administered over the course of 24 hours with one spray of a composition comprising 21.4% w/w CBD per day. In alternative embodiments, an individual human or animal requiring a daily dose of 30 mg CBD is administered over the course of 24 hours with four sprays of a composition comprising 5.35% w/w CBD.

Anxiety and Disorders Associated with Anxiety

In one aspect of the present invention, the composition or pharmaceutical formulation is for use in the treatment or prevention of anxiety or a disorder associated with anxiety.

In another aspect of the invention, there is provided a method of treating or preventing anxiety or a disorder associated with anxiety, wherein the method comprises administering to a patient in need thereof a composition or pharmaceutical formulation as described above.

In another aspect of the invention, there is provided the use of a composition or pharmaceutical formulation as described above for use in a method of manufacturing a medicament for the treatment or prevention of anxiety or a disorder associated with anxiety.

Anxiety is characterized by a number of both mental and physical symptoms, sometimes with no apparent explanation. Common mental symptoms include apprehension, fear of losing control, fear of going “crazy”, fear of pending death, impending danger or uneasiness. Common physical symptoms include dizziness, light-headedness, chest pain, abdominal pain, indigestion, nausea, increased heart rate or diarrhoea. Other symptoms may include insomnia, panic attacks, headaches, palpitations or fatigue.

Examples of disorders associated with anxiety include those as defined by the International Classification of Diseases (ICD), in particular classification no. ICD-10 F40-48.

Examples of disorders associated with anxiety include generalized anxiety disorder (also known as chronic anxiety), social anxiety disorder, panic disorder or episodic paroxysmal anxiety, obsessive compulsive disorder, post-traumatic stress disorder, phobic anxiety disorders, social phobias, specific phobias such as agoraphobia, claustrophobia or animal phobias, acute stress disorder, separation anxiety disorder, selective mutism, substance or medication-induced anxiety disorder, anxiety disorders due to other medical conditions, anxiety disorders without a specific cause, depression, atypical depression, recurring subclinical anxiety, persistent anxiety, chronic subclinical anxiety, persistent anxiety, anxious depression, neurosis, healing avoidance anxiety, dissociative anxiety, mixed anxiety and depressive disorder, severe stress, adjustment disorders, dissociative disorders such as dissociative amnesia, dissociative fugue, dissociative stupor, trance and possession disorders, dissociative motor disorders, dissociative convulsions, dissociative anaesthesia and sensory loss, mixed dissociative disorders, Ganser syndrome, multiple personality disorder and dissociative conversion disorder, somatoform disorders such as somatization disorder, undifferentiated somatoform disorder, hypochondriacal disorder, somatoform autonomic dysfunction and persistent somatoform pain disorder, and neurotic disorders such as neurasthenia, depersonalization-derealization syndrome, Dhat syndrome, occupational neurosis, psychasthenia, psychasthenic neurosis, psychogenic syncope.

It is to be understood that in some embodiments, the efficacy of the composition or pharmaceutical formulation in treating anxiety or a disorder associated with anxiety is tested by using the assay described in Examples 1, 3 and 5.

In one aspect of the present invention, there is provided the use of a composition or pharmaceutical composition as described above for the treatment or prevention of non-clinical anxiety.

In one aspect of the invention, there is provided a method of treating or preventing non-clinical anxiety, wherein the method comprises administering to a patient in need thereof a composition or pharmaceutical formulation as described above.

In another aspect of the invention, there is provided the use of a composition or pharmaceutical formulation as described above for the manufacture of a medicament for the treatment or prevention of non-clinical anxiety.

### EXAMPLES

The following Examples involve the use of a composition referred to as the “test product”. This test product is a composition comprising CBD and terpenes, with ≤0.001% w/w 9-tetrahydrocannabinol (THC), and falls within the terpene:cannabinoid range of between 1:3 and 1:60 w/w.

### Example 1—Test Product Dose Response Pilot

Doses used:
Result 1=water control (or, in Experiment 1, trial 2, 40 mg/L test product)
Result 2=vehicle=0.04% methanol (see below for discussion of the concentration)
Result 3=0.5 mg/L test product
Result 4=5 mg/L test product
Result 5=10 mg/L test product
Result 6=20 mg/L test product

### Experimental Method

Individual 5 dpf zebrafish larvae were placed in 250 μl fish water in each well of a 48 well plate, and were allowed to habituate for 20 minutes (min). Then, 50 μl of pre-diluted test product (total volume in carrier oil) was added to each well and the plate was subsequently transferred to the Noldus DanioVision® tracking system. Serial dilutions were prepared from a 1:2 test product: methanol mix (1:1 test product: methanol did not mix).

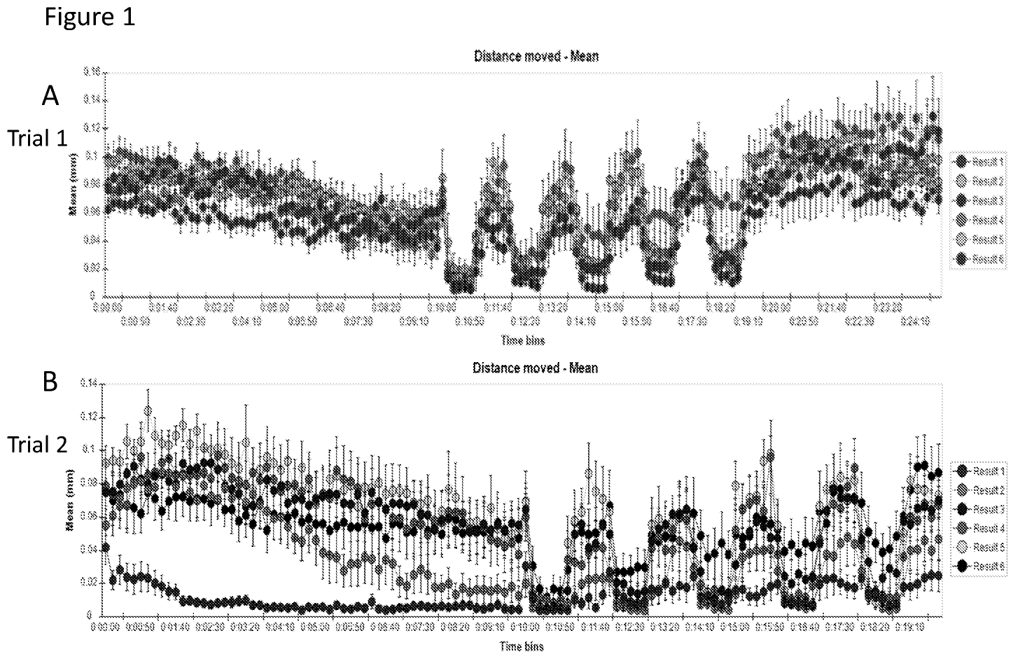

Experiment 1 (FIGS. 1A and 1B): Show mean distance travelled (mm) per 10 seconds. 10 min in dark followed by 5×1 min forced light dark transitions and then 5 minutes dark (same regimen for trials 1 and 2). For trial 2, there was no recording of the last 5 min of locomotion (in the dark). In addition, for trial 2, Result 1 is 40 mg/L test product rather than water (which is Result 1 in trial 1).

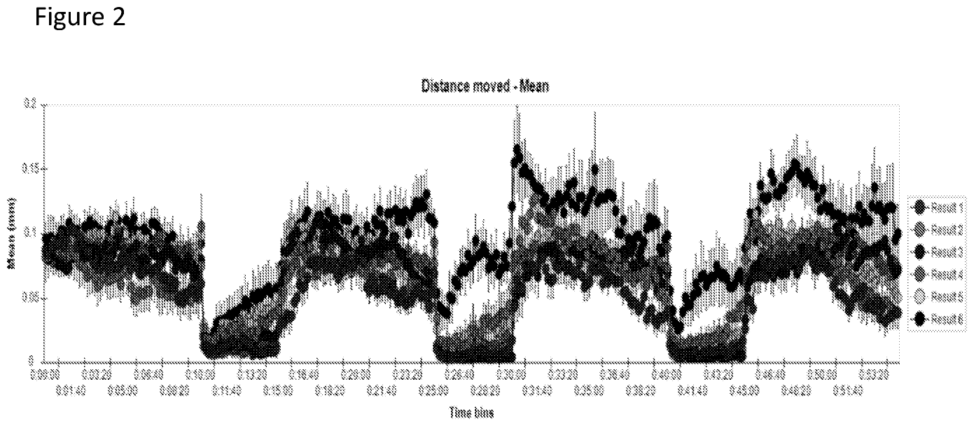

Experiment 2 (FIG. 2): Shows mean distance travelled (mm) per 10 seconds. 10 min in dark followed by 3×5 min light, 10 min dark forced light dark transitions, 10 min dark.

### DISCUSSION

It is clear that the test product has an effect over and above the methanol or carrier. In particular, the test product has a dose-dependent effect on locomotion in the light and dark phases.

### Example 2

Experimental methods used were the same as for Example 1 above.

### Results

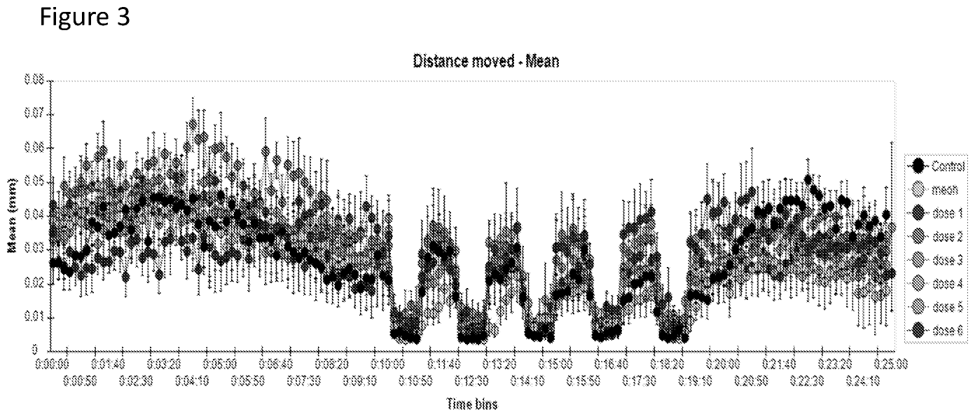

FIGS. 3 to 6 show the mean distance travelled (mm) per 10 seconds.

### FIG. 3—Test Product Dose Response (5×1 Min Forced Light Dark Transitions)

Dose 1=0.1 mg/L test product
Dose 2=0.5 mg/L test product
Dose 3=1 mg/L test product
Dose 4=5 mg/L test product
Dose 5=10 mg/L test product
Dose 6=20 mg/L test product

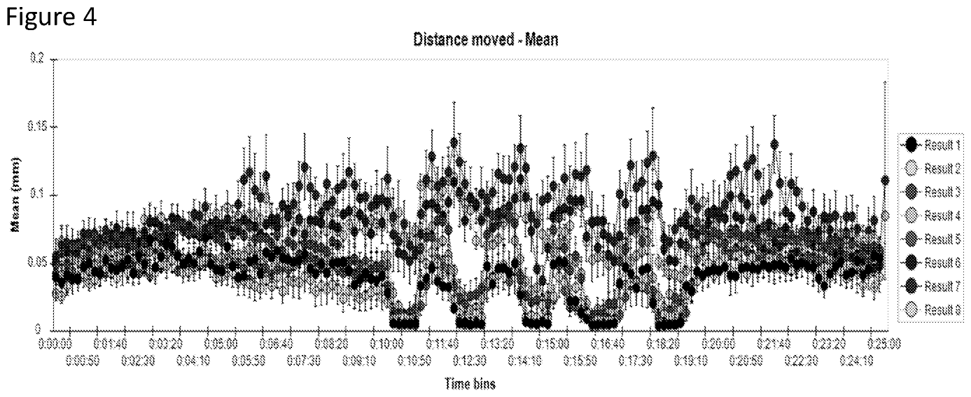

### FIG. 4—Fraction 1 in Methanol (5×1 Min Forced Light Dark Transitions)

Result 1=control (fish water)
Result 2=0.001% methanol
Result 3=0.5 mg/L Fraction 1
Result 4=1 mg/L Fraction 1
Result 5=5 mg/L Fraction 1
Result 6=10 mg/L Fraction 1
Result 7=15 mg/L Fraction 1
Result 8=20 mg/L Fraction 1

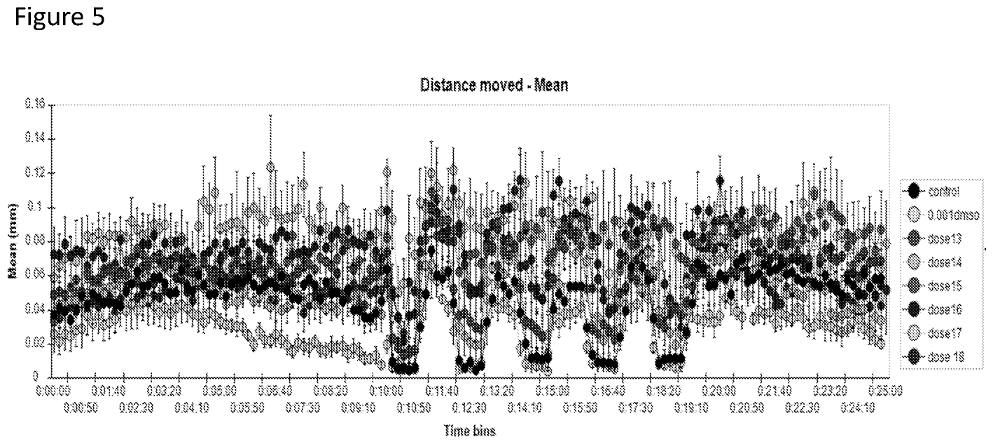

### FIG. 5—Fraction 1 in DMSO (5×1 Min Forced Light Dark Transitions)

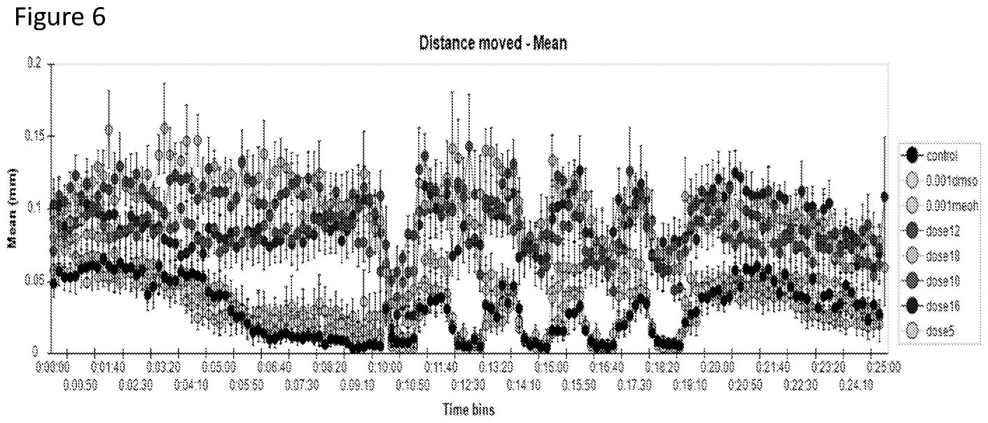

Control
0.001% DMSO
Dose 13=0.5 mg/L
Dose 14=1 mg/L
Dose 15=5 mg/L
Dose 16=10 mg/L
Dose 17=15 mg/L
Dose 18=20 mg/L
   FIG. 6—Fraction 1 in Methanol Vs. DMSO Vs. Test Product (5×1 Min Forced Light Dark Transitions)
Control
0.001% DMSO
0.001% methanol
Dose 5=10 mg/L test product
Dose 12=20 mg/L Fraction 1 in methanol
Dose 18=20 mg/L Fraction 1 in DMSO
Dose 10=10 mg/L Fraction 1 in methanol
Dose 16=10 mg/L Fraction 1 in DMSO

### Example 3

Aim: to assess the effects of extracts of hemp oil on zebrafish larval locomotor behaviour and response to forced light dark transition (commonly used to assess anxiolytic effects of compounds: Larvae freeze when the light is turned on, increased rate of recovery or loss of freezing response is considered indicative of reduced anxiety).
Hypothesis: that hemp oil extracts have anxiolytic activity.
Experiment: 5×1 min and 3×5 min Forced light/dark transition.

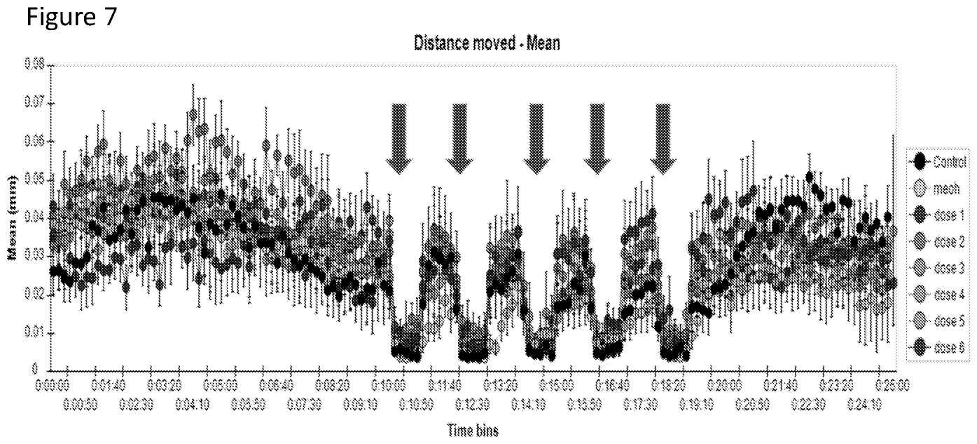

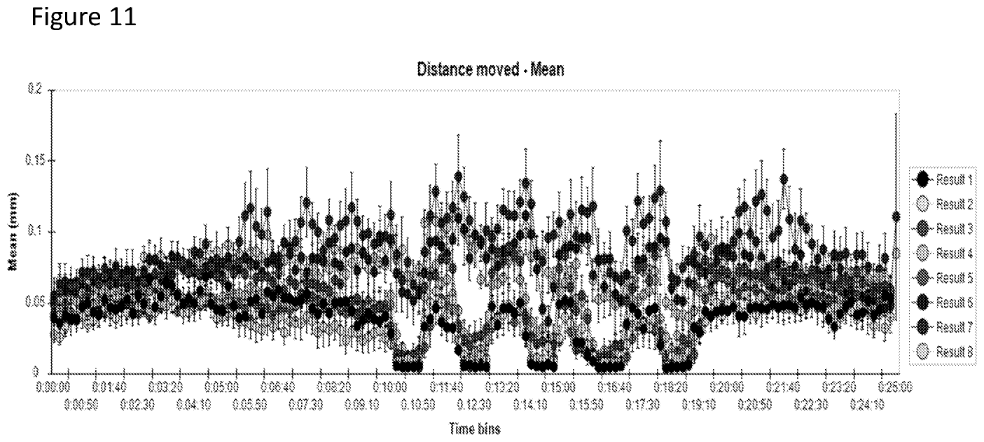

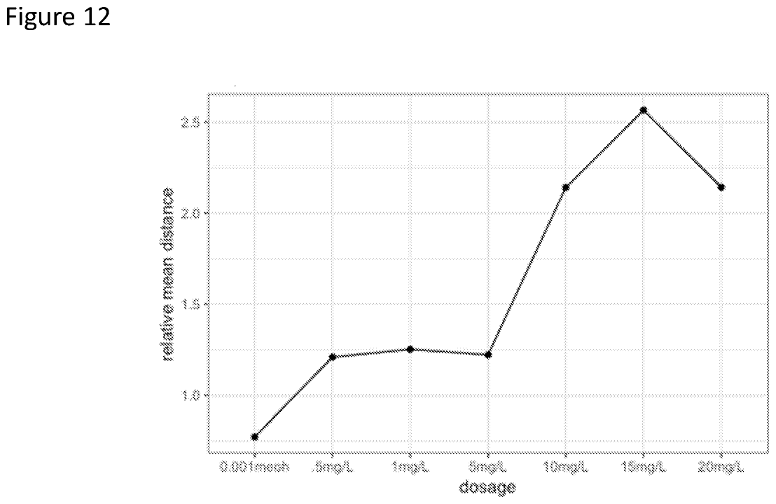

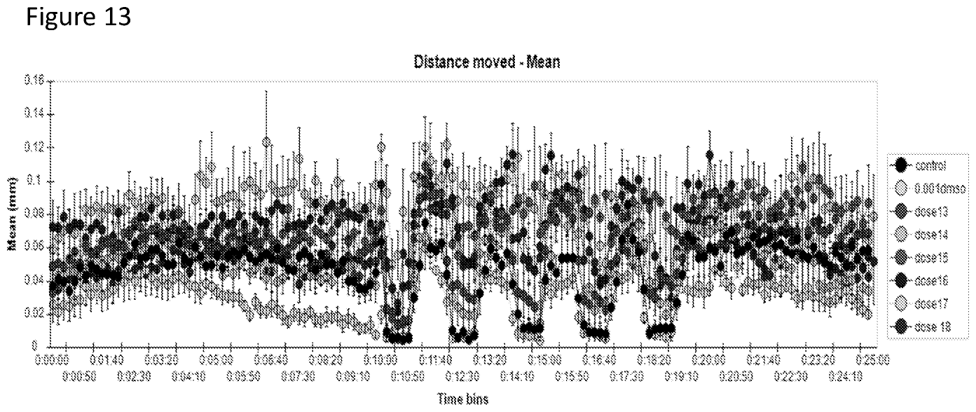

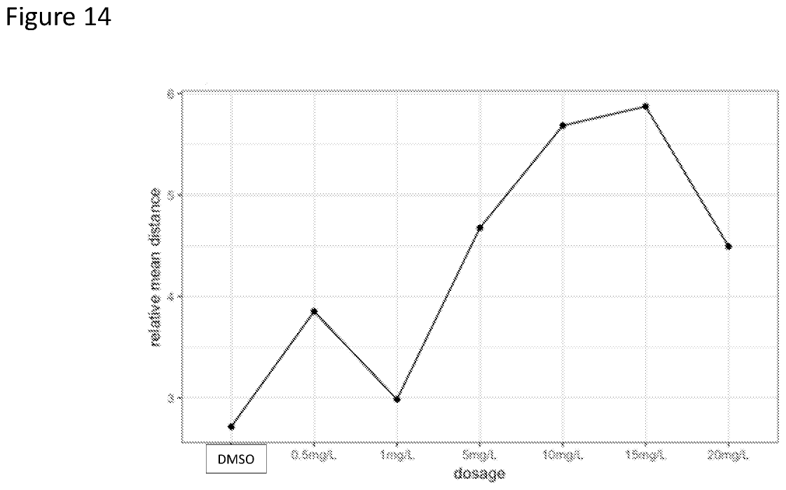

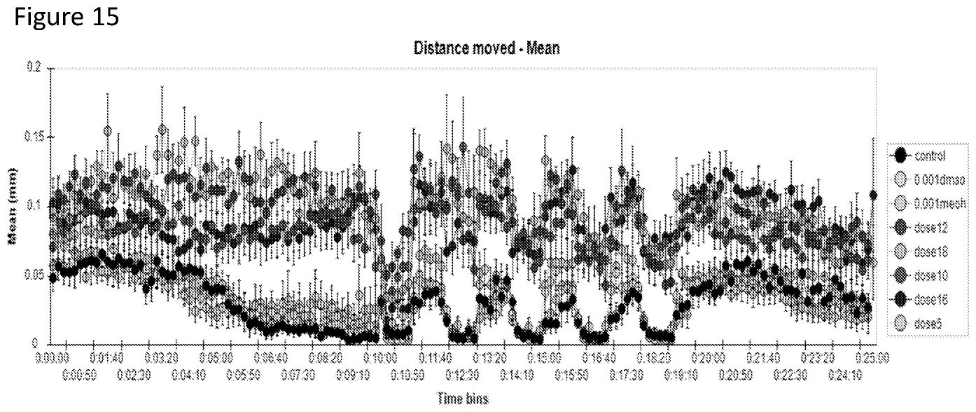

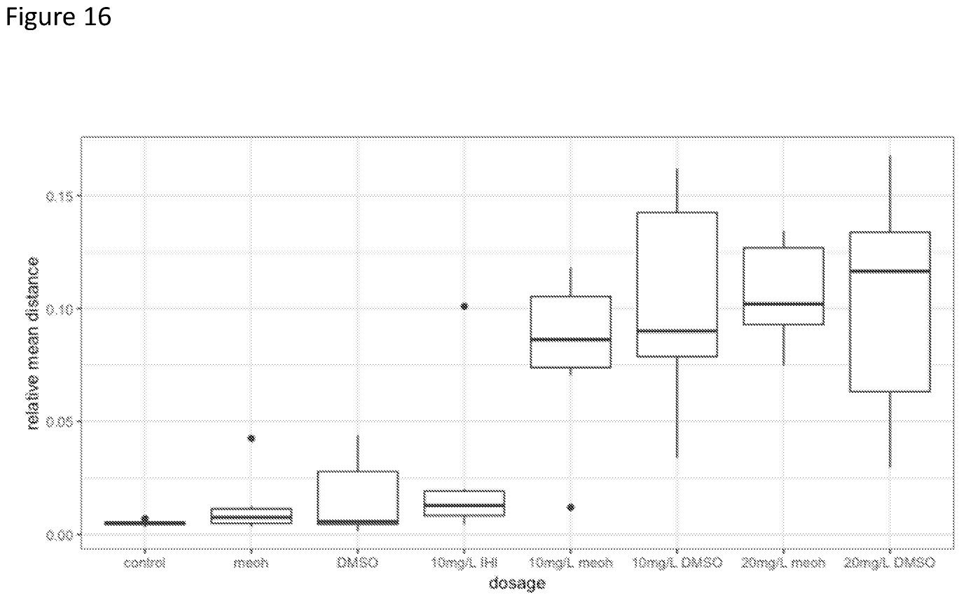

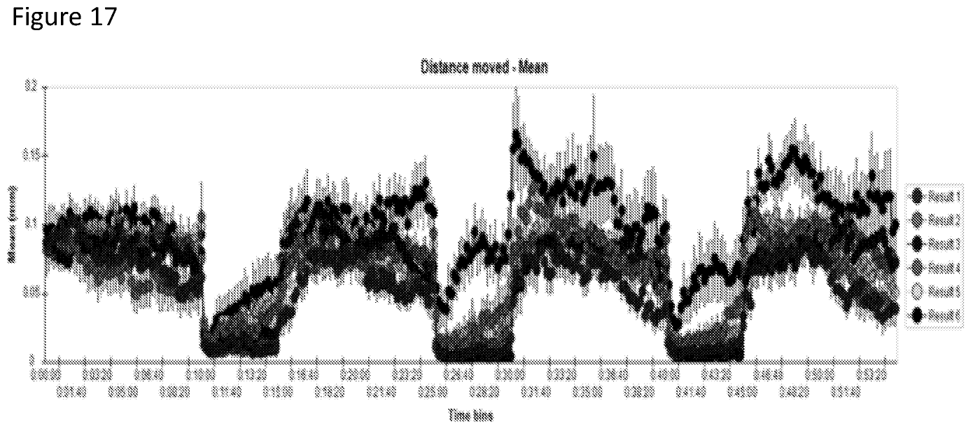

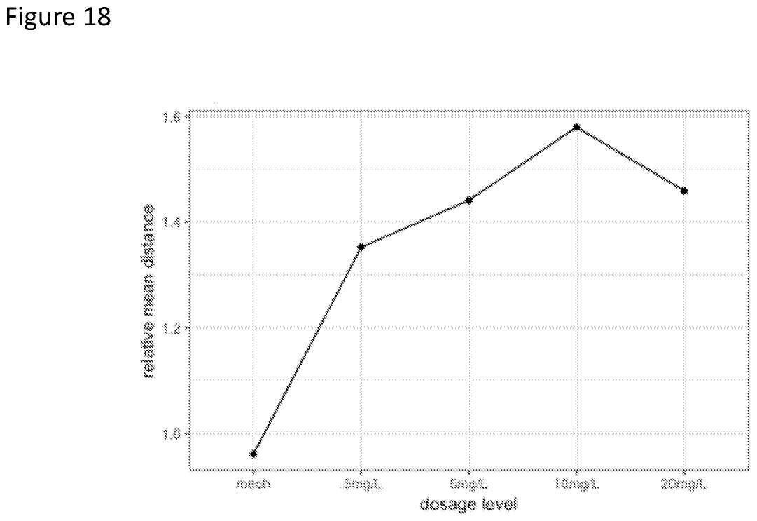

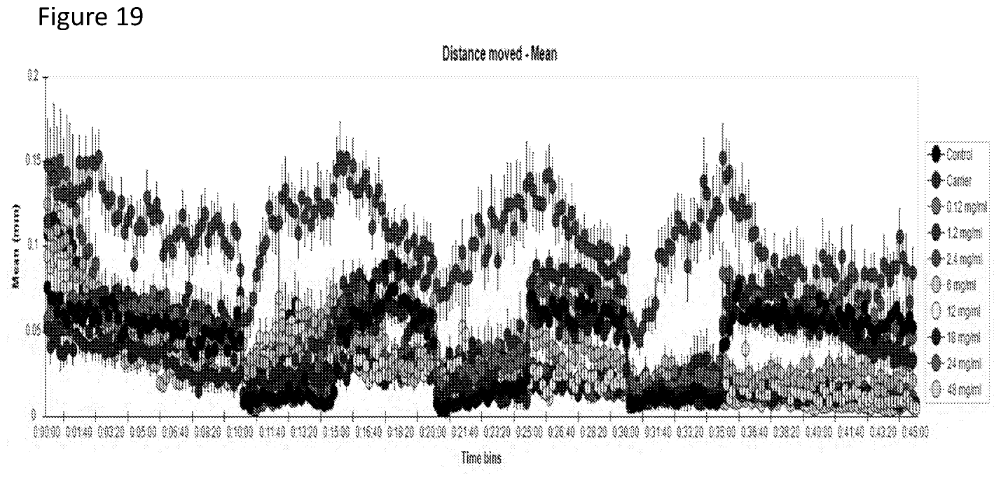

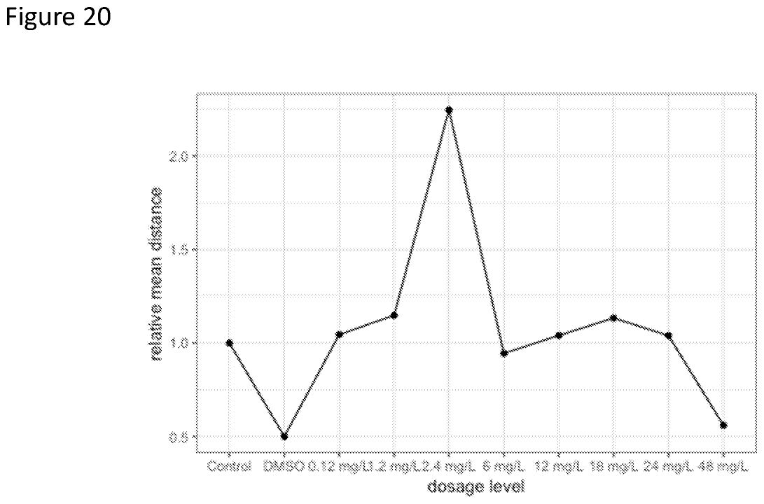

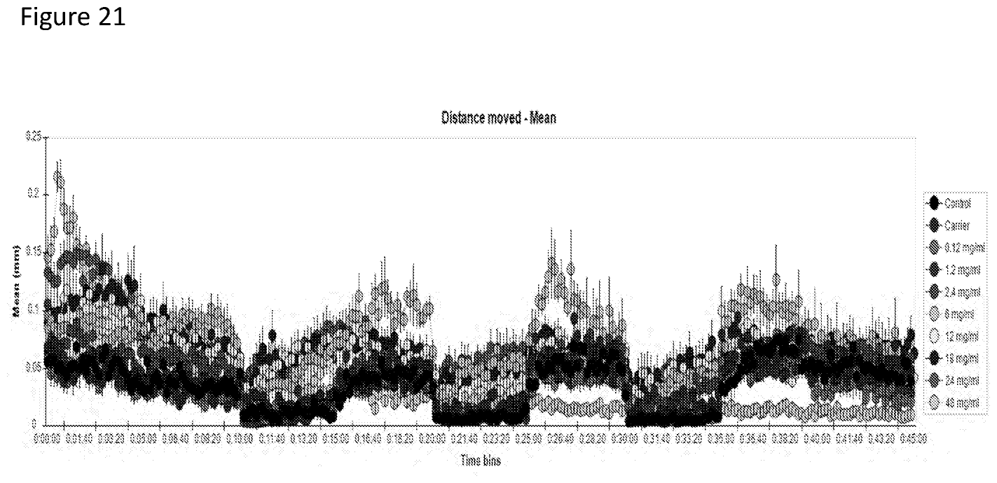

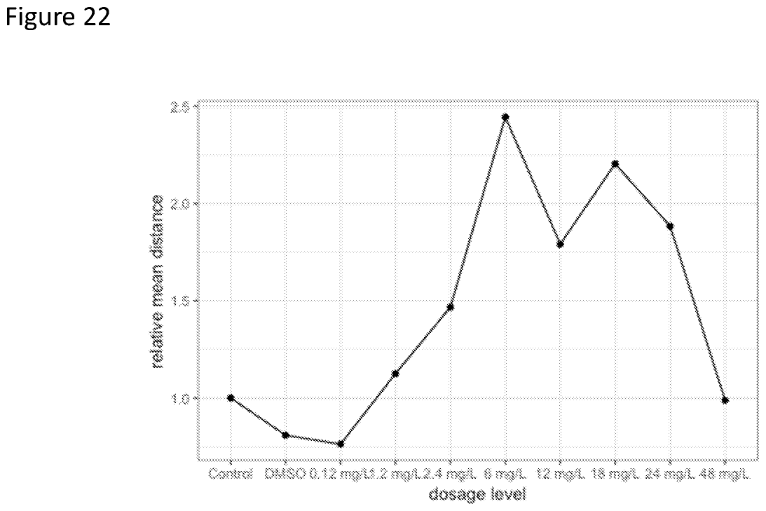

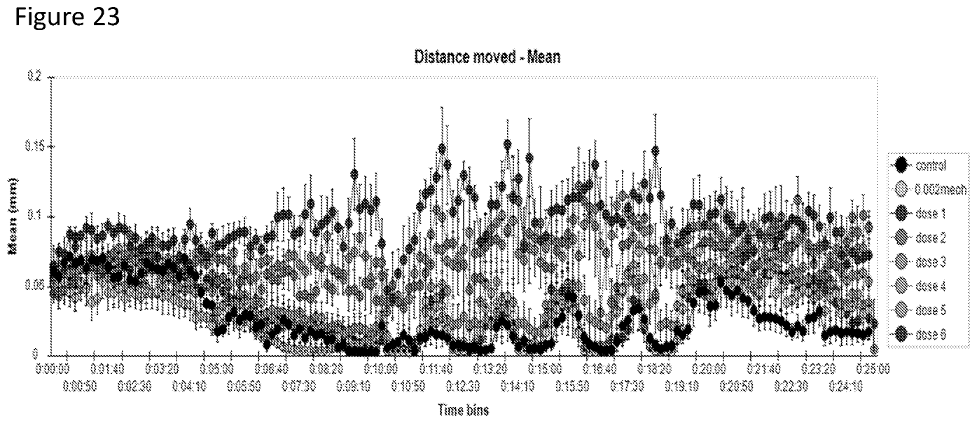

Individual zebrafish larvae were placed in 250 μl fish water in each well of a 48 well plate. The larvae were allowed to habituate for 30 minutes. Then an increasing concentration of the fraction of interest (test product, Fraction 1, 2 or 3) was added in a volume of 50 μl of carrier and the plate was then immediately transferred to the Noldus DanioVision® tracking system. Locomotor responses in response to forced light dark transition over a period of up to 40 min were recorded (n=6-12 in all cases).
Experiment 1 (FIGS. 7-10)=test product in methanol 0.1-20 mg/L. Assay timing: 10 min locomotion in dark, 5×1 min forced light dark transitions (light on for 1 min, then off for 1 min, repeated 5×) 10 min locomotion.
Experiment 2 (FIGS. 11 and 12)=Fraction 1 in methanol 0.5-20 mg/L (5×1 min forced light/dark transition)
Experiment 3 (FIGS. 13 and 14)=Fraction 1 in DMSO 0.5-20 mg/L (5×1 min forced light/dark transition)
Experiment 4 (FIGS. 15 and 16)=comparison of Fraction 1 in methanol vs. DMSO vs. test product 10 mg/L and 20 mg/L (5×1 min forced light/dark transition). For FIG. 15, dose 12=20 mg/L Fraction 1 in methanol, dose 18=20 mg/L Fraction 1 in DMSO, dose 10=10 mg/L Fraction 1 in methanol, dose 16 is 10 mg/L Fraction 1 in DMSO, dose 5=10 mg/L test product.
Experiment 5 (FIGS. 17 and 18)=test product in methanol (3×5 min forced/light transition).
Experiment 6 (FIGS. 19 and 20)=test product in DMSO (3×5 min forced light/dark transition).
Experiment 7 (FIGS. 21 and 22)=Fraction 2 in 0.001% DMSO (3×5 min forced light/dark transition).
Experiment 8 (FIGS. 23 to 26)=Fraction 2 and Fraction 3 (5×1 min forced light/dark transition). In DMSO vs. methanol.

### Notes:

1) The variable label ‘condition’ was used for control/methanol/dosage strengths
2) For all the statistical analyses, the time bins were converted to seconds. In each experiment, the statistical methodology used is the same as that explained under Example 5.

For Experiments 1 and 5, the test product solution was prepared by mixing 100 μl test product oil with 200 μl methanol to make a stock solution, which was then diluted to make working solutions. It was assumed that the solution partitioned equally when working out the concentrations. For Experiment 6, test product was diluted 1:1 in DMSO, and this seemed to mix completely.

### Experiment 1—Test Product in Methanol:

FIG. 7 shows the mean distance travelled (mm) per 10 seconds. Dose 1 to Dose 6:0.1 mg/L, 0.5 mg/L, 1 mg/L, 5 mg/L, 10 mg/L, 20 mg/L (40 mg/L killed the larvae). Arrows indicate light periods (note that locomotion decreases when the light comes on). Mean±/−SEM. N=6

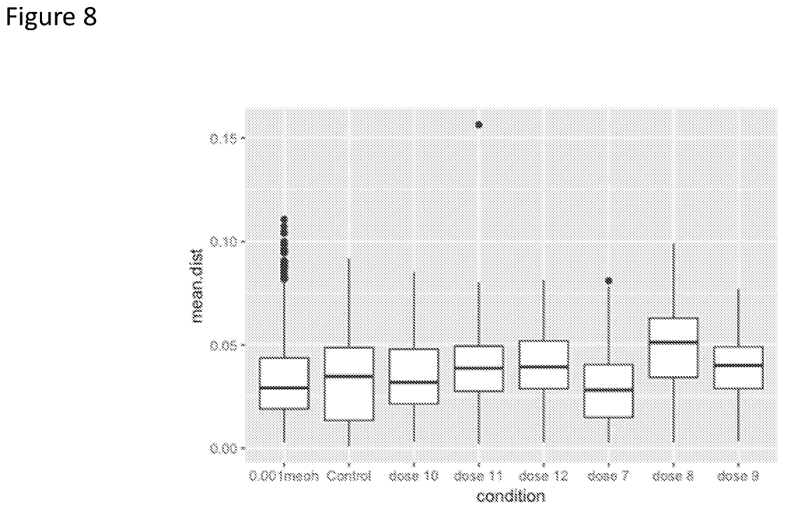

FIG. 8 shows the mean distance travelled (mm) per 10 seconds across the 10 minutes of the baseline period for Fraction 1 in methanol. Doses 7-12 are Fraction 1 at 0.5 mg/L, 1 mg/L, 5 mg/L, 10 mg/L, 15 mg/L and 20 mg/L in methanol.

### Baseline Comparison—Experiment 1:

A linear mixed-effect model (LMM) was run with mean distance per time bin (i.e. 10 seconds), the response variable and condition and time as fixed effects, and individual identity (ID) as a random effect. This trial led to the unexpected result that, at baseline, condition (i.e. dosage) was not a significant predictor of mean distance (Likelihood Ratio Test (LRT)=6.9, p=0.439).

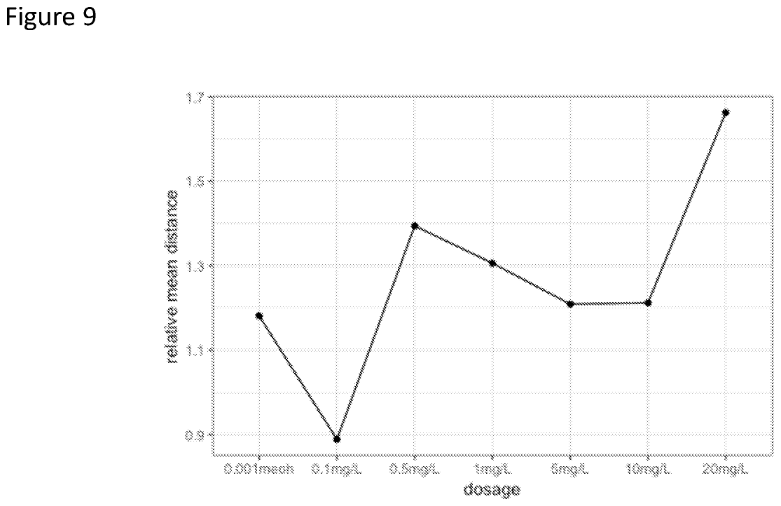

It seems very likely this is due to individual variability (when this model is run without individual as a random effect, it is highly significant, but then suffers from massive pseudoreplication). Interestingly, the interaction between condition and time is significant at this comparison for baseline (LRT=33.1, P<0.001), which suggests that the patterns of movement over the course of the 10-minute acclimation period are different, perhaps due to drug absorption. For this reason, the last minute of the baseline period (i.e. before the light/dark period) was used to calculate the dose response curve, shown in FIG. 9.

### Dose Response Curve for Last Minute of Baseline Period—Experiment 1:

FIG. 9 shows the mean distance travelled over the last 60 seconds of the baseline period for Experiment 1.

### Light/Dark Period—Experiment 1:

An LMM was run with same response variable, fixed effects, and random effect on the subset of data starting from the first light event. Analysing this period combined also yields similar results to baseline: the interaction between time and condition was significant (LRT=20.4, p<<0.001), but the individual predictors are not. This again implies that condition is a significant predictor of movement patterns during periods of light/dark.

### Slopes—Experiment 1:

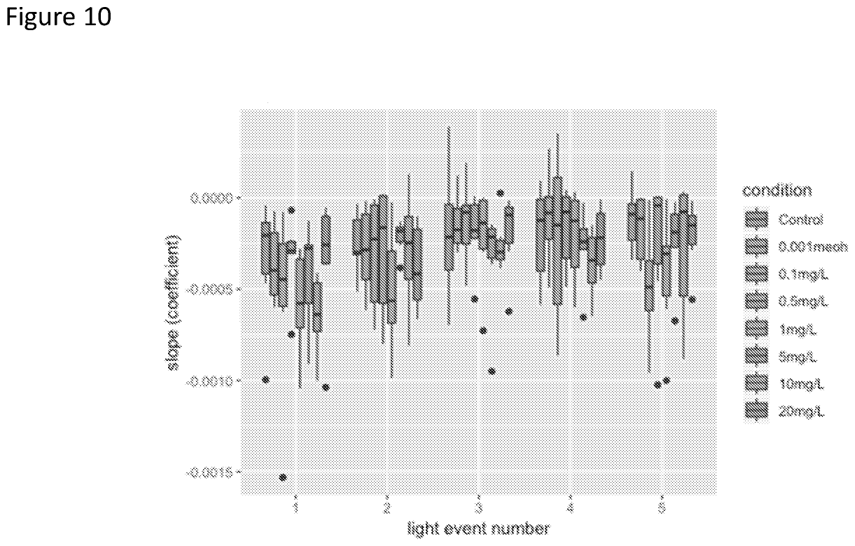

FIG. 10 shows the slope of the mean distance/time regression for each individual larvae per condition in each light number. The slope represents the rate of recovery during the light period. An increased rate of recovery is consistent with a reduction in anxiety.

The linear regression coefficient (i.e. the slope of the relationship between mean distance moved and time) was calculated for each individual in each condition in each light event number. Then an LMM with slope as the response variable, light event number and condition as the fixed effects, and individual ID as a random effect, was run. For this trial, we see that condition is not a significant predictor of slope (LRT=4.8, 0.68), although light event number is (LRT=35.3, p<0.001); however, their interaction was not (and impressively so, LRT=27.5, p=0.94).

### Experiment 2—Fraction 1 in 0.001% Methanol:

FIG. 11 shows the mean distance travelled (mm) per 10 seconds. Results 1 to 8: control, 0.001% methanol, 5 mg/L, 1 mg/L, 5 mg/L, 10 mg/L, 15 mg/L and 20 mg/L of Fraction 1.

### Baseline Comparison—Experiment 2:

An LMM was run as with Experiment 1. Condition was a significant predictor of mean distance during baseline. Additionally, so is the interaction between condition and time (p<<0.001). For this reason, the last minute of the baseline period (i.e. before the light/dark period) was used to calculate the dose response curve.

FIG. 12 shows the Dose Response curve for last minute of baseline period for Experiment 2 (the mean distance travelled over the last 60 seconds of the baseline period).

### Light/Dark Period—Experiment 2:

An LMM was run as with Trial 1. As with baseline, condition on its own is a significant predictor of mean distance during the light/dark period, and again, so is the interaction between condition and time (p<<0.001). This implies that the condition (i.e. dosage) had an effect on movement beyond straightforward elevation/depression of locomotion.

### Slopes—Experiment 2:

Performed as with Experiment 1. Condition was a significant predictor (p=0.020), although light event number was not (p=0.119). Their interaction was not (p=0.281). i.e. the rate of recovery from the startle differs with dose. From the graph of movement per 10 seconds (FIG. 11), the low doses increase rate of recovery-usually interpreted as anxiolytic.

### Experiment 3—Fraction 1 in 0.001% DMSO:

FIG. 13 shows the mean distance travelled (mm) per 10 seconds across the experimental time course. Results: control, 0.001% DMSO, dose 13-18=0.5 mg/L, 1 mg/L, 10 mg/L, 15 mg/L and 20 mg/L of Fraction 1.

### Baseline Comparison—Experiment 3:

An LMM was run as with Experiment 1. It comes out sensibly, with condition being a significant predictor of mean distance during baseline. More importantly, so is the interaction between condition and time (p<<0.001).

FIG. 14 shows the Dose Response curve for last minute of baseline period for Experiment 3 (the mean distance travelled over the last 60 seconds of the baseline period).

### Light/Dark Period for Experiment 3:

An LMM was run as with Experiment 1. As with baseline, condition on its own is a significant predictor of mean distance during the light/dark period, and again, so is the interaction between condition and time (p<<0.001). This implies that the condition (i.e. dosage) had an effect on movement beyond straightforward elevation/depression of locomotion.

### Slopes for Experiment 3:

Performed as with Experiment 1. Condition was a significant predictor of slope (p=0.019), as was light event number (p<0.001). Their interaction was not (p=0.223).

Experiment 4—Comparison of Fraction 1 in Methanol Vs. Fraction 1 in DMSO Vs. Test Product:

FIG. 15 shows the mean distance travelled (mm) per 10 seconds across the experimental time course. Results control, 0.001 DMSO, 0.001 methanol, Dose 5=10 mg/L test product in DMSO, Dose 12=20 mg/L Fraction 1 in methanol, Dose 18=20 mg/L Fraction 1 in DMSO, Dose 10=10 mg/L Fraction 1 in methanol, Dose 16=10 mg/L Fraction 1 in DMSO.

### Baseline Comparison for Experiment 4:

An LMM was run as with Experiment 1. Condition is a significant predictor of mean distance during baseline (p<<0.001). More importantly, so is the interaction between condition and time (p<<0.001).

FIG. 16 shows the mean distance travelled over the last 60 seconds of the baseline period for Experiment 4.

### Light/Dark Period for Experiment 4:

An LMM was run as with Experiment 1. As with baseline, condition on its own is a significant predictor of mean distance during the light/dark period, and again, so is the interaction between condition and time (p<<0.001). This implies that the condition (i.e. dosage) had an effect on movement beyond straightforward elevation/depression of locomotion.

### Slopes for Experiment 4:

Performed as with Experiment 1. Condition was not significant predictor of slope (p=0.12); neither was light event number was not (p=0.925), nor was their interaction (0.690).

### Experiment 5—Test Product in 0.02% Methanol (3×5 Min Light, 10 Min Dark):

### Test Product 3×5 Min Forced Light Dark Transition Preliminary Data Analysis:

FIG. 17 shows the mean distance travelled (mm) per 10 seconds across the experimental time course.

Result 1 is control, Result 2 is methanol, Result 3 is 0.5 mg/L, Result 4 is 5 mg/L, Result 5 is 10 mg/L and Result 6 is 20 mg/L test product.

### Notes:

1) Assumed test product partitioned equally into the methanol when working out concentrations (but see DMSO results)
2) I used the variable label ‘condition’ for control/me-OH/dosage strengths
3) I converted time to seconds

### Baseline Comparison for Experiment 5:

A linear mixed-effect model (LMM) was run with mean distance, the response variable and condition and time as fixed effects, and individual ID as a random effect. Condition (i.e. dosage) was not a significant predictor of mean distance (LRT=9.1, p=0.104), but the interaction between condition and time was (LRT=160.6, p<<0.001). This suggests that the patterns of movement over the course of the 10-minute acclimation period are different. For this reason, the last minute of the baseline period (i.e. before the light/dark period) was used to calculate the dose response curve.

FIG. 18 shows the Dose Response curve for the last minute of the baseline period.

### Light/Dark Period for Experiment 5:

An LMM was run with same response variable, fixed effects, and random effect on the subset of data starting from the first light event. Condition was a significant predictor of mean distance (LRT=23.4, p<0.001), as was the interaction between condition and time (LRT=88.6, p<<<0.001).

### Slopes for Experiment 5:

The linear regression coefficient (i.e. the slope of the relationship between mean distance moved and time) was calculated for each individual in each condition in each light event number. An LMM was run with slope as the response variable, light event number and condition as the fixed effects, and individual ID as a random effect. For this trial, condition is not a significant predictor of slope (LRT=1.3, p=. 935), but light event number is (LRT=189.6, p<0.001); their interaction was not (LRT=0.5, p=0.9918).

### Experiment 6—Test Product in 0.001% DMSO 3×5 Min Forced Light/Dark Transition:

FIG. 19 shows the mean distance travelled (mm) per 10 seconds across the experimental time course. Control, carrier=0.001% DMSO, test product concentration range 0.12-48 mg/L.

### Baseline Comparison:

A linear mixed-effect model (LMM) was run with mean distance, the response variable and condition and time as fixed effects, and individual ID as a random effect. Condition (i.e. dosage) was a significant predictor of mean distance (LRT=47.1, p<<0.001), as was the interaction between condition and time (LRT=418.1, p<<0.001) This suggests that the patterns of movement over the course of the 10-minute acclimation period are different. For this reason, the last minute of the baseline period (i.e. before the light/dark period) was used to calculate the dose response curve.

FIG. 20 shows the Dose Response curve for the last minute of the baseline period

### Light/Dark Period:

An LMM was run with same response variable, fixed effects, and random effect on the subset of data starting from the first light event. Condition was a significant predictor of mean distance (LRT=102.4, p<<<0.001), as was the interaction between condition and time (LRT=880.1, p<<<0.001).

### Slopes:

The linear regression coefficient (i.e. the slope of the relationship between mean distance moved and time) was calculated for each individual in each condition in each light event number. An LMM was run with slope as the response variable, light event number and condition as the fixed effects, and individual ID as a random effect. For this trial, we see that condition is not a significant predictor of slope (LRT=10.3, p=0.329), but light event number is (LRT=14.5, p<0.001); their interaction was not (LRT=3.8, p=0.925).

NB: test product diluted in DMSO-appears to partition into the DMSO more than methanol.

### Experiment 7—Fraction 2 in 0.001% DMSO:

FIG. 21 shows the mean distance travelled (mm) per 10 seconds across the experimental time course. Control (fish water), carrier=0.001% DMSO, fraction 2 concentration range 0.12-48 mg/L.

### Baseline Comparison:

An LMM was run as with Experiment 1. It comes out sensibly, with condition being a significant predictor of mean distance during baseline (LRT=60.4, p<0.001). So is the interaction between condition and time (LRT=965.8, p<<0.001).

FIG. 22 shows the Dose Response curve for the last minute of the baseline period.

### Light/Dark Period:

An LMM was run as with Experiment 1. As with baseline, condition on its own is a significant predictor of mean distance during the light/dark period (LRT=58.9, p<<0.001), and again, so is the interaction between condition and time (LRT=206.1, p<<0.001). This implies that the condition (i.e. dosage) had an effect on movement beyond straightforward elevation/depression of locomotion.

### Slopes:

Performed as with Experiment 1. Condition was a significant predictor (LRT=35.6, p<0.001), but light event number was not (LRT=2.9, p=0.09). Their interaction, however, was not significant (LRT=5.6, p=0.778).

Experiment 8—Fraction 2 and Fraction 3. 5×1 Min Forced Light Dark Transition (fLDT):

### Fraction 2:

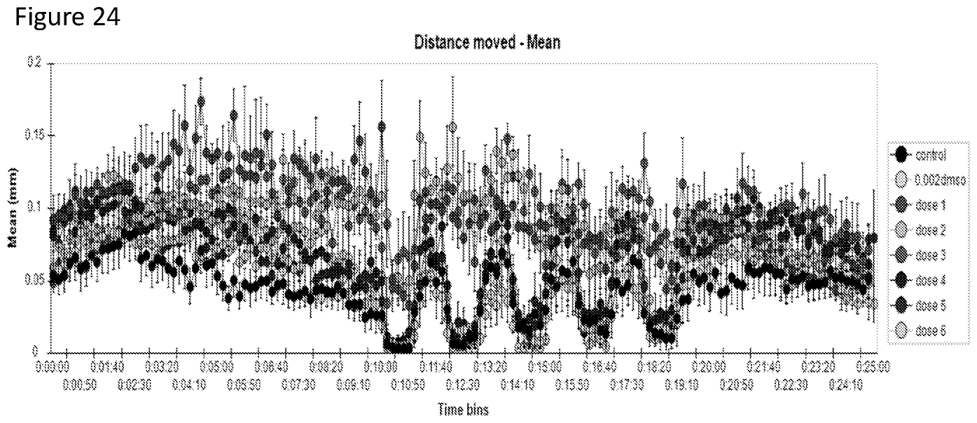

FIGS. 23 and 24 show mean distance travelled (mm) per 10 seconds across the experimental time course. 10 minutes dark, 5×1 min light, I min dark, 10 min dark. Results for FIG. 23=control (fish water), 0.002% methanol, 0.5 mg/L, 1.0, 2.0, 5.0 mg/L, 10 mg/L and 20 mg/L. Results for FIG. 24=control (fish water), 0.002% DMSO, 0.5 mg/L, 1.0, 2.0, 5.0 mg/L, 10 mg/L and 20 mg/L.

### Fraction 3:

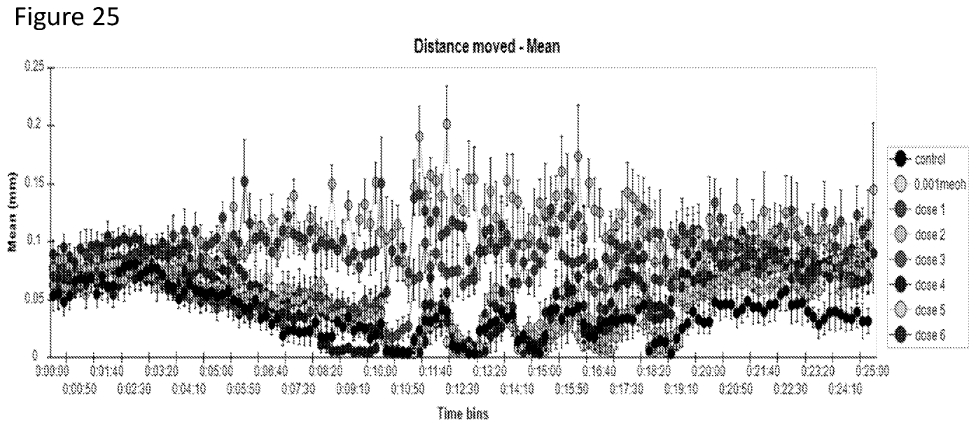

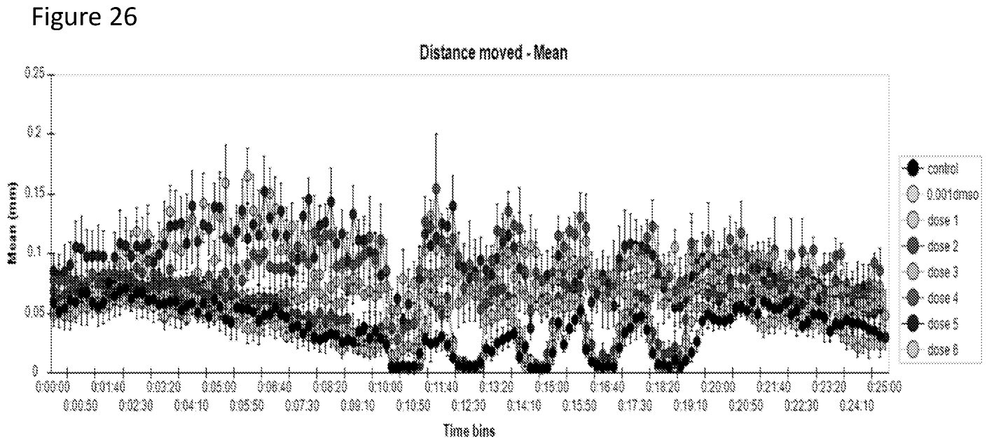

FIGS. 25 and 26 show mean distance travelled (mm) per 10 seconds across the experimental time course. 10 minutes dark, 5×1 min light, I min dark, 10 min dark. Results for FIG. 25=control (fish water), 0.001% methanol, 0.5 mg/L, 1.0, 2.0, 5.0 mg/L, 10 mg/L and 20 mg/L. Results for FIG. 26=control (fish water), 0.001% DMSO, 0.5 mg/L, 1.0, 2.0, 5.0 mg/L, 10 mg/L and 20 mg/L.

Results in methanol were compared with the results in DMSO for Fraction 2 and 3.

The same model was used as described previously (LMM, mean distance response variable, condition and time fixed effects, individual ID random effect) but with trial number as an additional fixed effect. It does indeed come out significant (LRT=24.5, p>>0.001). This implies that solvent has an effect on the way the drug absorbs/affects locomotion/(whatever is correct in this context)

### Summary Conclusions

0.001% methanol or DMSO alone have no effect on larval behaviour in this assay. 0.001% DMSO was a more efficient solvent for test product (which we assume explains the much sharper D/R curve for test product in DMSO). Similarly, the solvent used has a significant effect on the response to Fraction 2. Therefore we intend to conduct all future analysis in DMSO.

All compounds tested to date (test product, Fraction 1-3) cause a dose-dependent increase in locomotion, followed by inhibition of locomotion/death at high doses. When using 3×5 min transitions, test product at a low concentration (in methanol) appeared to increase rate of recovery from startle (see Experiment 5 trace) but this did not reach statistical significance. Fraction 2 had a dose dependent effect on recovery rate. An increased rate of recovery is consistent with an anxiolytic activity.

### Example 4

### Notes:

All the tests were done with 5 dpf larvae unless stated otherwise.
A forced light/dark transition test was used. (10 min dark-(5 min light-5 min dark)*3-5 min dark)
The dose response curve was made as follows;
   The locomotor activity of the larvae at the last min of acclimation (min 9-10 in the assay), was taken
   The mean for each larvae over this period of time was calculated
   Then the mean of each condition was calculated
   The means plotted are relative to the control, control was set at 1

### Fraction 1:

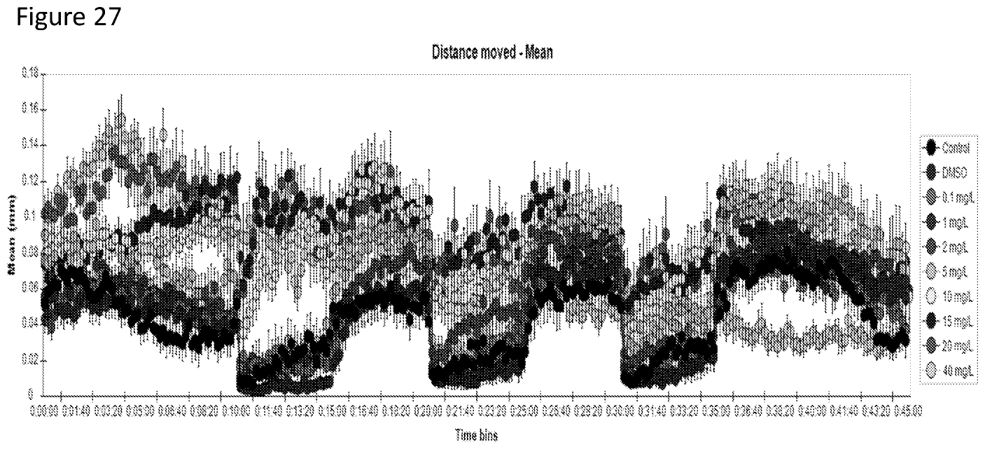

FIG. 27 shows the mean distanced travelled (mm) per 10 seconds across the experimental time course.

Test consist of 3 plates (n=15), gathered over 2 days.

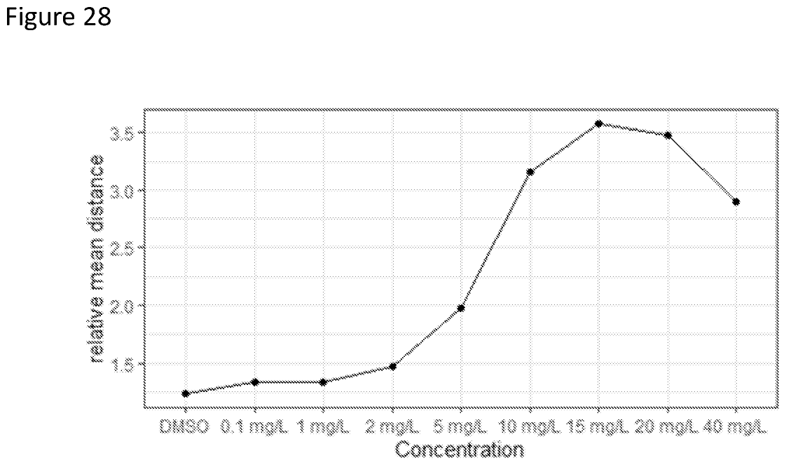

FIG. 28 shows the dose response curve

### Fraction 2:

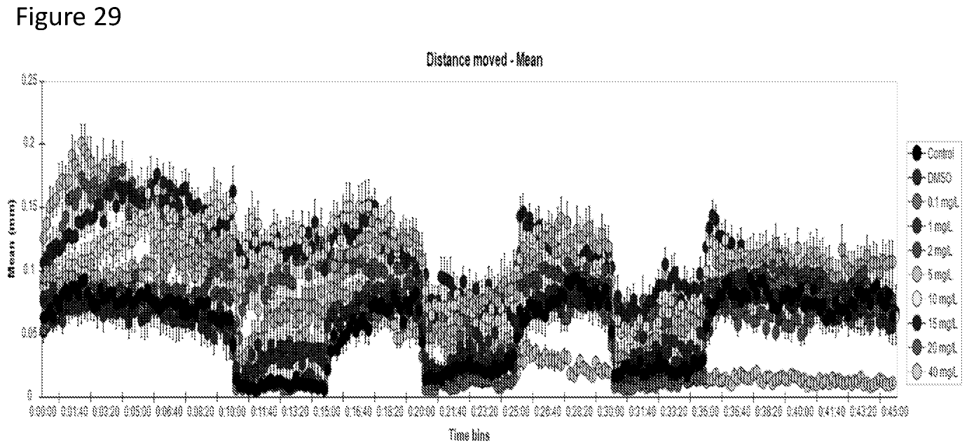

FIG. 29 shows the mean distanced travelled (mm) per 10 seconds across the experimental time course.

Test consist of 3 plates (n=15), gathered over 2 days.

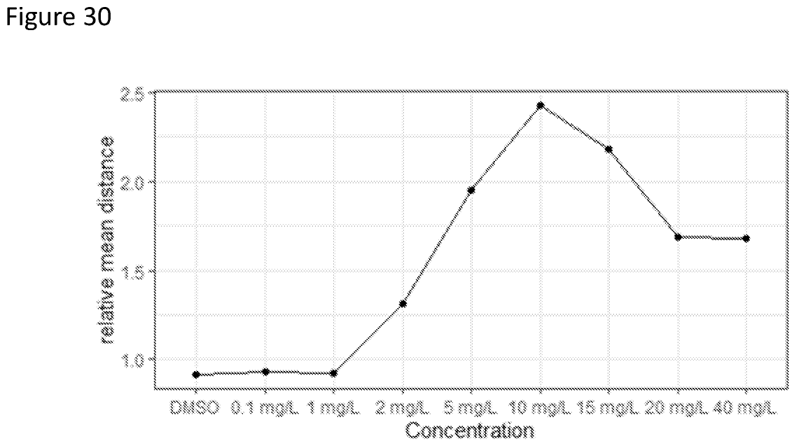

FIG. 30 shows the dose response curve of Fraction 2.

### Fraction 3:

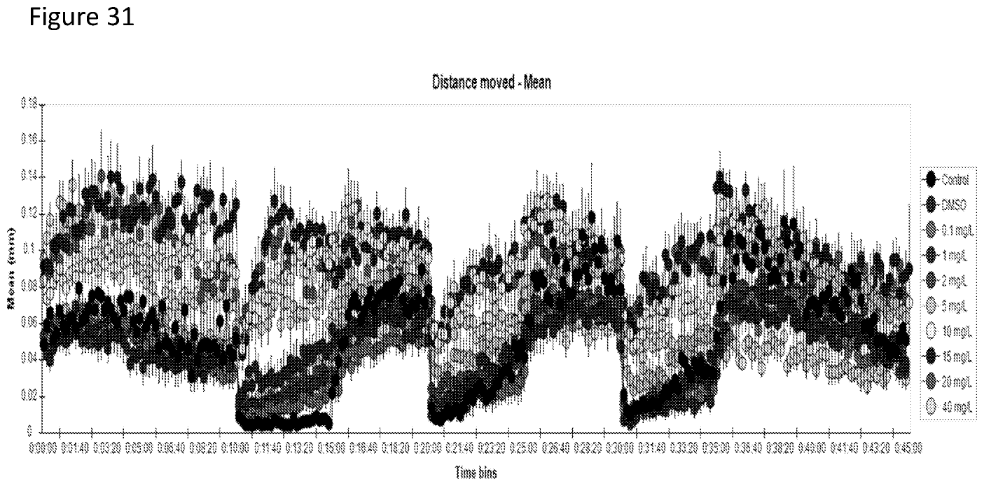

FIG. 31 shows the mean distanced travelled (mm) per 10 seconds across the experimental time course.

Test consist of 3 plates (n=15), gathered over 2 days.

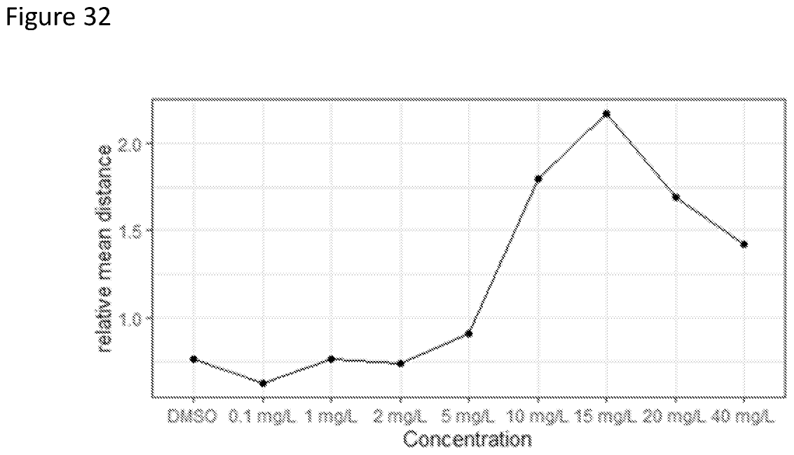

FIG. 32 shows the dose response curve for Fraction 3.

### Summary Fraction 1-3:

These concentrations seem to cover the dose response curve well. Ideally a concentration should be chosen where there is little to no effect on locomotor, while there still is an effect on anxiety/stress. We expect that this concentration lies around the 5 mg/L for all three fractions. Possibly around 7.5 mg/L for fraction 1 and 3 and for fraction 2 either 2 mg/L or 5 mg/L (or in between).

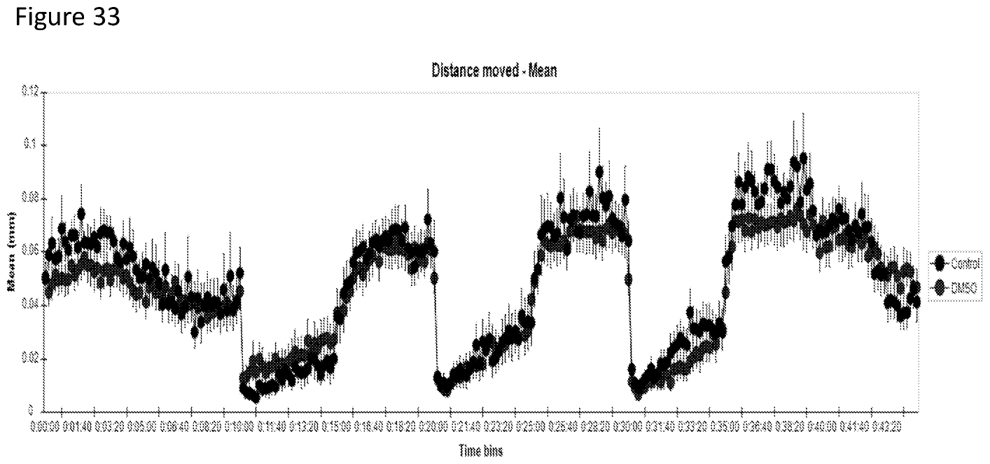

From the graphs, it seems to be that there might be a difference between DMSO and fishwater, however the graph shown in FIG. 33 combines the controls (only fishwater) and the DMSO of Fraction 1 and Fraction 3 as they have the same concentration of DMSO. Although no statistical tests have been performed there seems to be no difference between the control and DMSO in locomotor, freezing or recovery.

### Test Product:

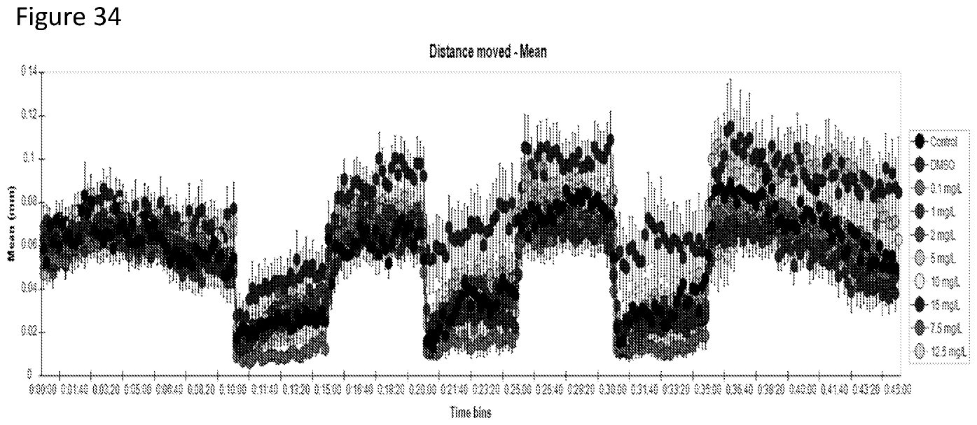

FIG. 34 shows the mean distanced travelled (mm) per 10 seconds across the experimental time course.

Test consist of 3 plates (n=15), gathered over 2 days.

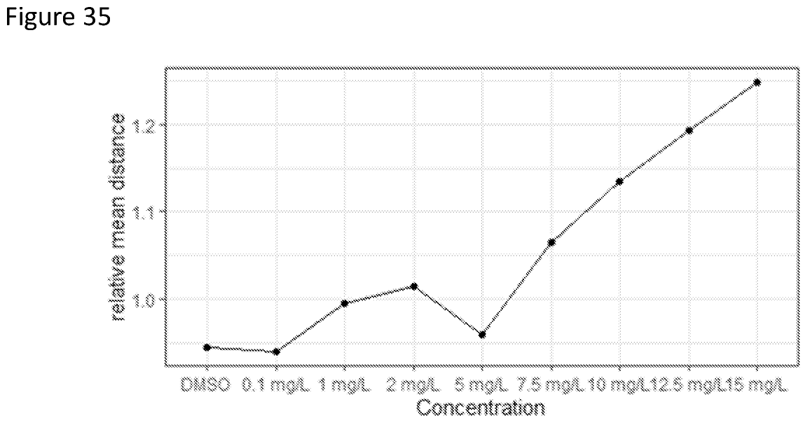

FIG. 35 shows the dose response curve for the test product.

### Summary of Test Product:

These concentrations of test product don't cover the dose response curve as well. A concentration of possibly 20 mg/L could be added to show the downward curve at the end of the concentration; however, we know that concentrations of 20 mg/L and higher kill the larvae by the end of the assay.

### Diazepam:

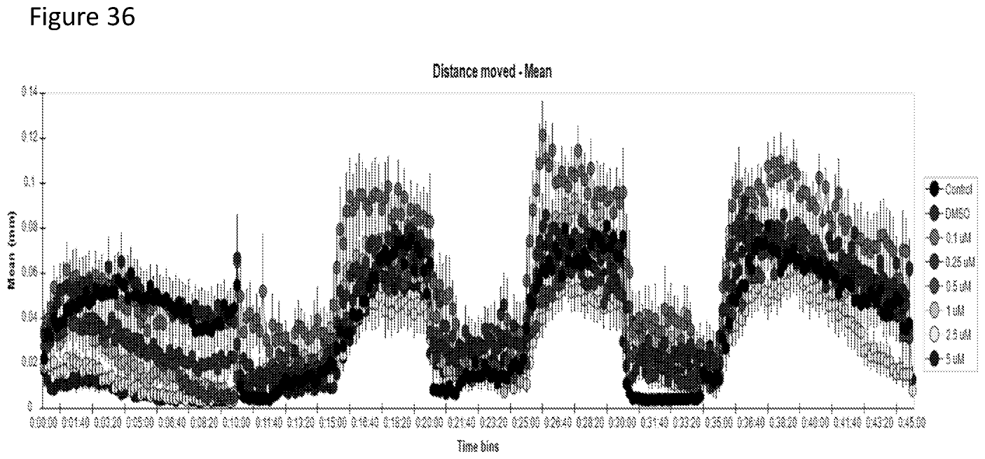

FIG. 36 shows the mean distanced travelled (mm) per 10 seconds across the experimental time course.

Testing of 2 plates (n=11), gathered on 1 day.

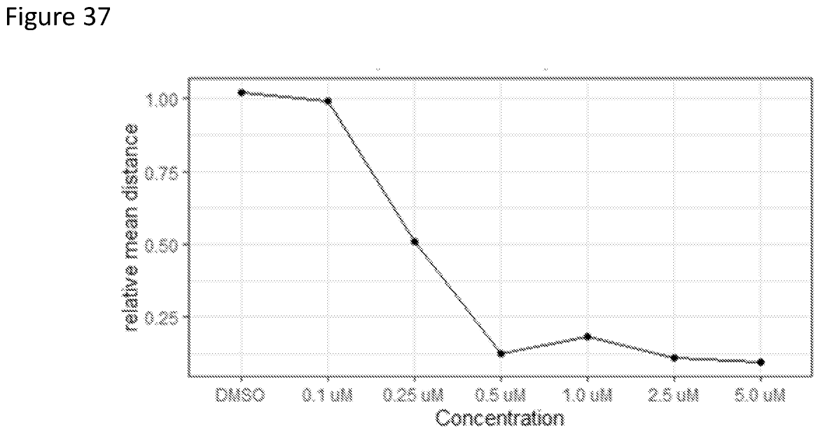

FIG. 37 shows the dose response curve for diazepam.

### Summary Diazepam:

Here we see clearly the impairment diazepam has on locomotor. There is an n=11, however some additional larvae had to be removed for this analysis, making the n for some concentrations lower.

### Ethanol:

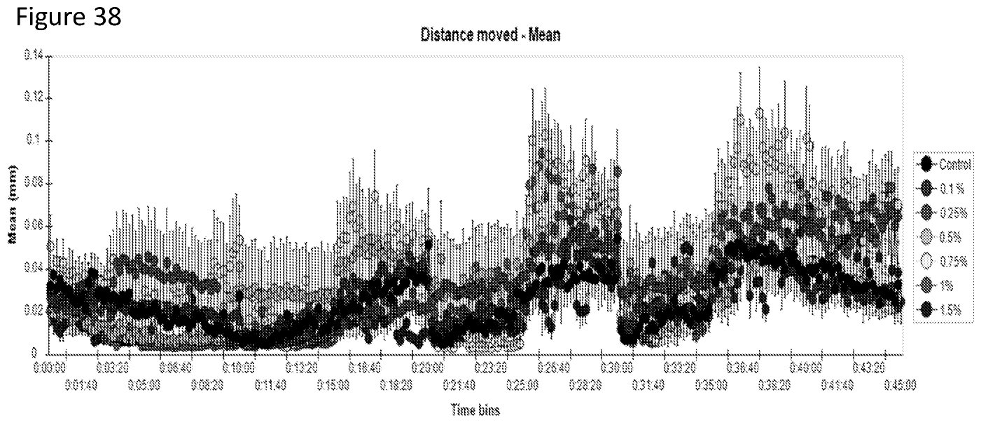

FIG. 38 shows the mean distanced travelled (mm) per 10 seconds across the experimental time course.

2 plates (n=14), gathered on one day.

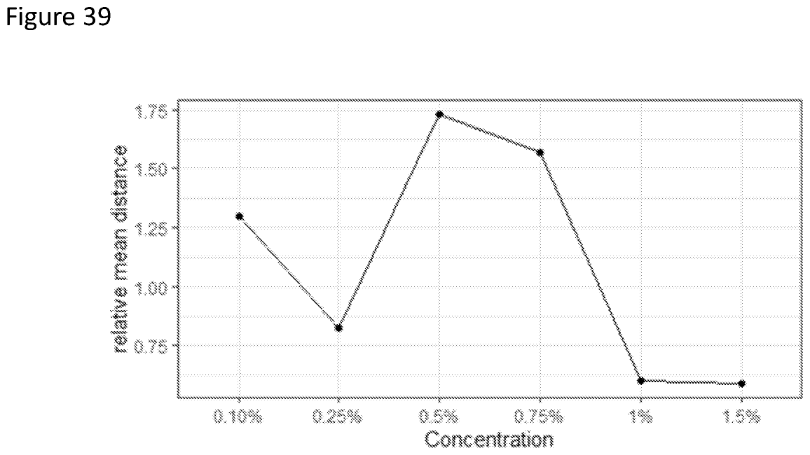

FIG. 39 shows the dose response curve for ethanol.

Looking at the data from the 2 assays that were done, the behaviour of these larvae seems erratic. Looking at the acclimation period, 0.25% ethanol larvae start moving erratically and while most groups freeze when turning on the light 0.5% ethanol starts moving more than before. Here another plate will be added to have a better statistical reliability. Some larvae had to be removed for from the assay. This will also influence these results and is another reason to add at least one plate.

4 dpf vs. 5 dpf:

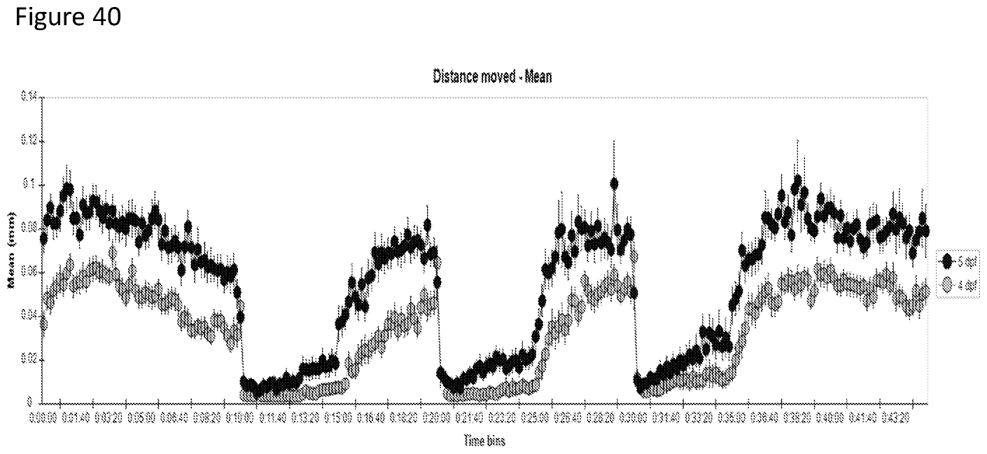

Here 4 dpf and 5 dpf larvae were compared on a single plate (n=24) (FIG. 40). Larvae at 4 dpf seem to move less. There does seem to be a freezing response, however due to them moving less, it is not as strong of a response as the 5 dpf larvae.

### Example 5

### Description of the Assay:

We used a forced light/dark transition assay to screen for possible behavioural/anxiolytic effects of four hemp extracts, in addition to two compounds with known anxiolytic and anxiogenic effects (diazepam and caffeine, respectively).

Our forced light/dark transition protocol was as follows. First, there was 10 minutes of total darkness (which we refer to as the baseline period; compounds were introduced at the beginning of the baseline period, and 10 minutes were allotted to allow for drug absorption and acclimation). Next, there was a period of five minutes of light exposure followed by five minutes of dark exposure, repeated three times. Finally, there was an additional five minutes of dark exposure.

### Statistical Methodology:

A variety of statistical techniques were used to address our hypotheses about the potential anxiolytic and anxiogenic effects of four compounds. In outline:
1) Overall models were carried out using linear mixed-effect models
2) Deliberate pairwise comparisons between controls and dosages of interest were carried out using appropriate two-sample comparison tests

All statistical analyses were carried out in R version 3.2.2 (R core developer team). Overall models were linear mixed-effects models (LMMs), and were fitted using the Ime4 package (Bates, Machler, Bolker, & Walker, 2015). Data were transformed as indicated to fit the assumptions of LMMs. Posthoc tests were performed using the multcomp package in R. Data distributions were initially assessed visually and model diagnostics were subsequently checked to assure appropriate fits.

Comparisons between patterns of activity during the light phases (i.e. rate of recovery during light phases, which indicates a possible anxiolytic or anxiogenic effect) were performed by analysing the slope of the linear regression function of individual mean distance vs time during light phases.

For pairwise comparisons on specific concentrations, nonparametric tests are used as indicated on non-normally distributed data.

### Control Analysis:

### Caffeine:

Caffeine is a stimulant anxiogenic which we assessed in our forced light/dark transition assay to analyse the manifestation of anxiogenic behaviour in this context.

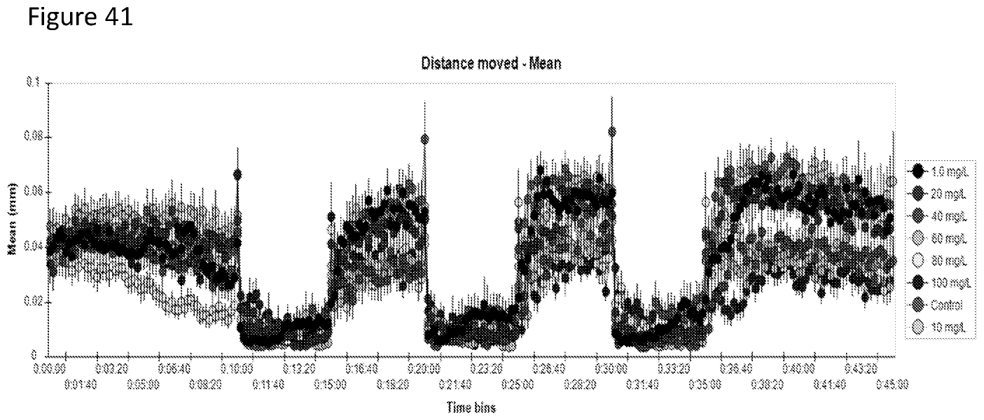

FIG. 41 shows the mean distance travelled (mm) per 10 seconds over the whole duration of the assay. The fish go to the bottom of the tank when the light is switched on. The effect is much the same for all doses of caffeine.

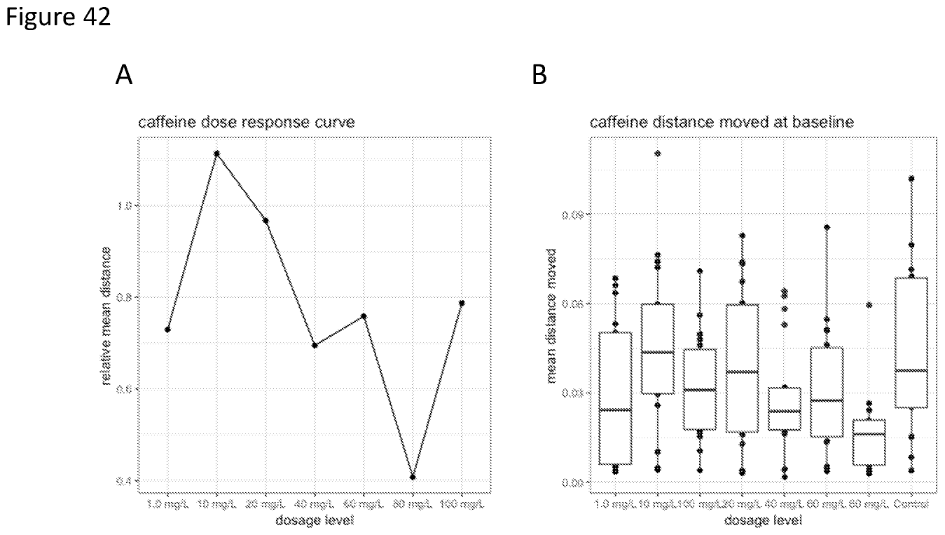

FIGS. 42A and 42B show the dose response curve and distance moved at baseline.

### Exposure Effects:

To assess differences in locomotion after ˜10 minutes of exposure to the drug, we analysed mean distances moved over one minute. We used a linear mixed-effects model (LMM) with individual mean distance moved (in 10 s bins) as the response variable, dosage as a fixed effect, and individual ID as a random effect. Dosage was a significant predictor of mean distance moved after 10 minutes of exposure (LMM: LRT=20.5, p=0.005).

Caffeine dosages had generally lower locomotion than control larvae, and a Tukey posthoc test and one dosage, 80 mg/L, had significantly lower locomotion.

### Locomotion During Light Phases:

To assess differences in locomotion both within light phases and between them, we used a linear mixed-effects model (LMM) with the log-transformed individual mean distance moved (in 10 s bins) as the response variable, the interaction between time within the light phase and dosage as a fixed effect, interaction between dosage and light phase number as a fixed effect, and individual ID as a random effect. Both fixed effects were significant predictors of mean distance moved (LMM: LRT=194, p<0.001 and LRT=127.8 and p<0.001, respectively), and caffeine dosage did, overall, have an effect on the distance moved. Dosages of 40 mg/L and greater were significantly different than the control in pairwise comparisons (Tukey post-hoc test). However, this effect was difficult to assess directly, as variation from the control happened within light phases and through differences in habituation to subsequent light phases.

### Diazepam:

Diazepam is a sedative anxiolytic which we assessed in our forced light/dark transition assay to analyse the manifestation of anxiolytic behaviour in this context.

### Experiment 1 (Diazepam):

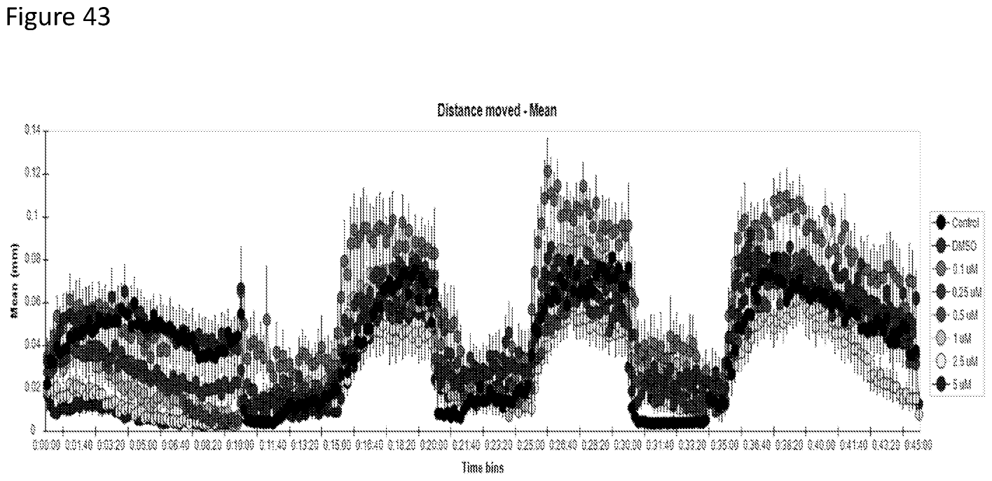

FIG. 43 shows the mean distance travelled (mm) per 10 seconds over the whole duration of the assay. Even the lowest dose of diazepam stops the fish moving to the bottom of the tank.

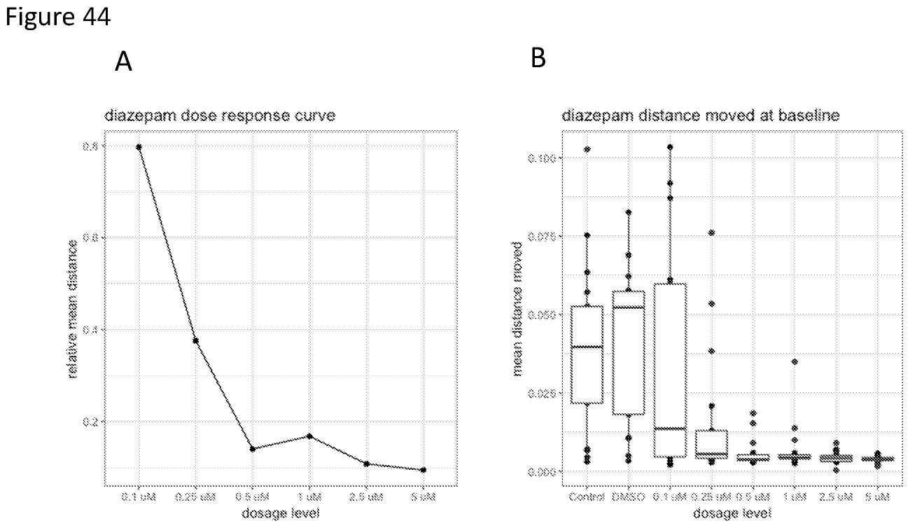

FIGS. 44A and 44B shows the dose response curve and distance moved at baseline.

### Exposure Effects:

To assess differences in locomotion after ˜10 minutes of exposure to the drug, we analysed mean distances moved over one minute. We used a linear mixed-effects model (LMM) with individual mean distance moved (in 10 s bins) as the response variable, dosage as a fixed effect, and individual ID as a random effect. This model was significant (LMM, LRT=63.5, p<0.001) and a Tukey post hoc test revealed that pairwise comparisons between the control fish and all dosages 0.25 μM and higher revealed that the diazepam-treated fish had significantly lower distance moved than the control fish.

### Locomotion During Light Phases:

To assess differences in locomotion both within light phases and between them, we used a linear mixed-effects model (LMM) with the log-transformed individual mean distance moved (in 10 s bins) as the response variable, the interaction between time within the light phase and dosage as a fixed effect, interaction between dosage and light phase number as a fixed effect, and individual ID as a random effect. Both fixed effects were significant predictors of mean distance moved (LMM: LRT=97.3, p<0.001, LRT=74.1, p<0.001).

To assess the rate of recovery during light phases (i.e. the slope of the linear regression of fish resuming movement after freezing), we used a Kruskal-Wallis test to compare the slope of the linear regression during the first light phase. This test is significant, with X=22.5, df=7, p-value=0.002); the diazepam treated larvae have a faster rate of recovery (i.e. a larger slope) during the first light phase. For a specific pairwise comparison, we compared DMSO (i.e. control) larvae with larvae in the 0.25 μM concentration; we chose 0.25 μM because it had significant effects on locomotion without indication of physical effects over the duration of the study. The diazepam-treated fish at 0.25 μM had a significantly higher slope, or rate of recovery, during light phases (Wilcox signed rank test, W=214, p=0.016). The faster rate of recovery following the stressful stimulus of the light demonstrates the anxiolytic effect of diazepam.

### Experiment 2 (Diazepam):

### Baseline-Locomotion:

A subset of minute 9 to 10 was made to isolate the baseline. A linear mixed model (LMM) with concentration as fixed effect and individual as random effect, and mean distance moved as response variable.

There was no significant difference found in mean distance moved in the baseline period (LRT 6.7032, p>0.05).

### Light Challenge:

A subset of data was made of minute 10 to 20 to isolate the light challenge. A linear mixed model (LMM) with concentration and the interaction with time as a fixed effect, individual as random effect, and mean distance moved as response variable.

There was a significant effect of concentration on mean distance moved over time (LRT=163.73, p<0.001). 2.5 μM was significantly different from the control (p<0.01) and thus increases movement over time faster than the control in the light challenge

### Dark Period:

A subset of data was made of minute 20 to 30 to isolate the dark period. A linear mixed model (LMM) with concentration and the interaction with time as a fixed effect, individual as random effect, and mean distance moved as response variable.

There was a significant effect of concentration on mean distance moved over time (LRT=185.83 p<0.001). 2.5 μM was significantly different from the control (p<0.001) and thus decreases movement over time faster than the control in the dark period.

The analysis of diazepam demonstrates the anxiolytic effect of diazepam.

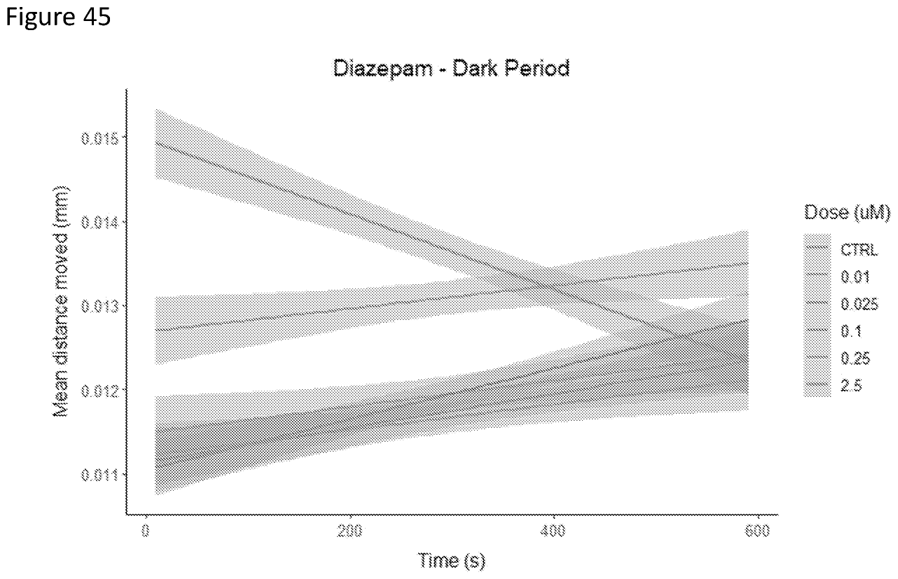

FIG. 45 shows the mean distance moved (mm) during the dark period (diazepam).

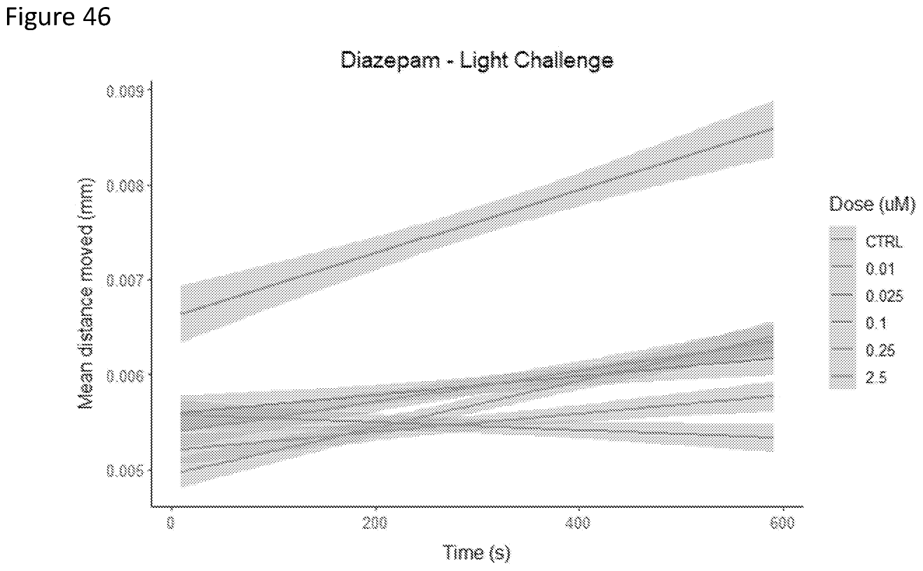

FIG. 46 shows the mean distance moved (mm) during the light challenge (diazepam).

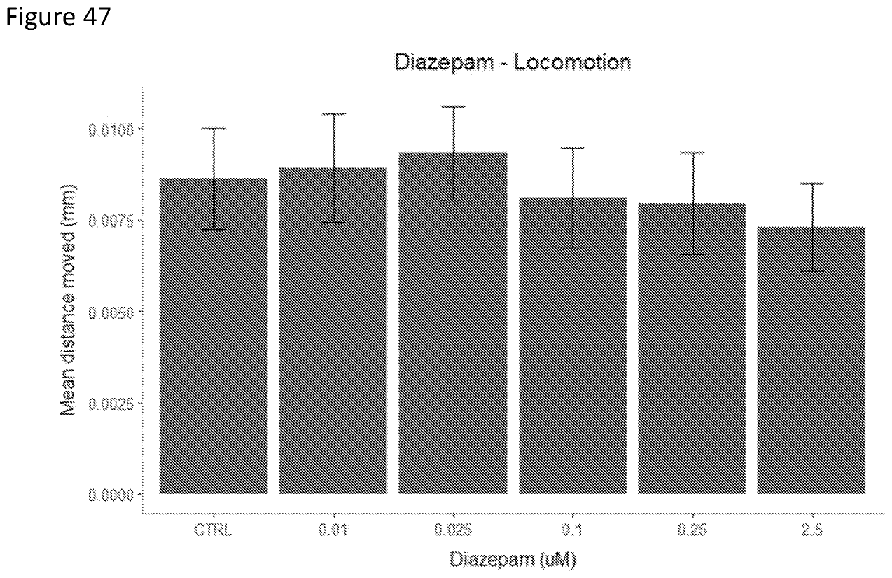

FIG. 47 shows the mean distance moved (mm) during the baseline period (diazepam).

### Fraction 1:

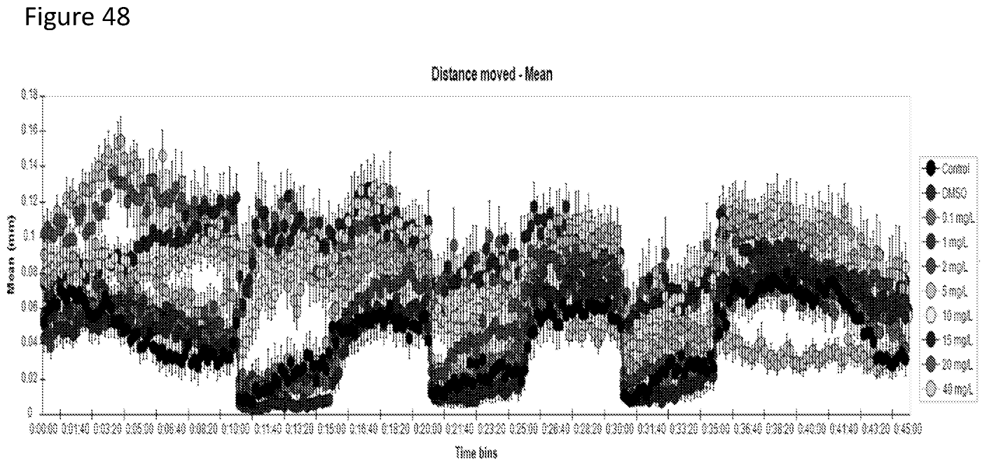

FIG. 48 shows the mean distance travelled (mm) per 10 seconds over the whole duration of the assay. There is perhaps a toxic effect at 40 mg/L.

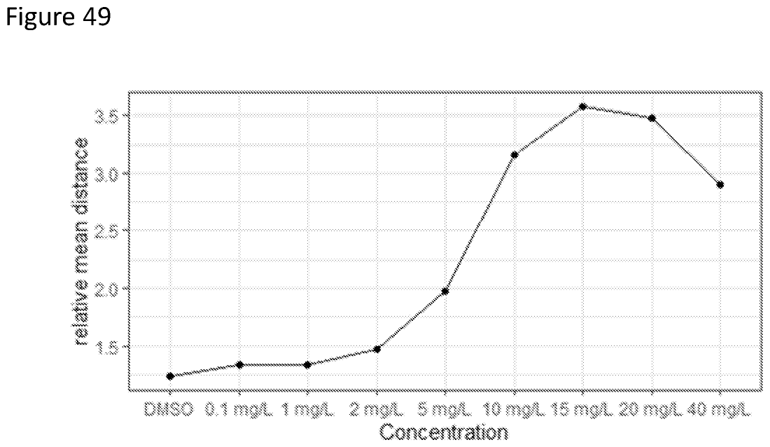

FIG. 49 shows the dose response curve of distance moved in the last minute of baseline.

### Exposure Effects:

To assess differences in locomotion after ˜10 minutes of exposure to the drug, we analysed mean distances moved over one minute. We used a linear mixed-effects model (LMM) with individual mean distance moved (in 10 s bins) as the response variable, dosage as a fixed effect, and individual ID as a random effect. Dosage was a significant predictor of mean distance moved after 10 minutes of exposure (LMM: LRT=69.5, p<0.001). A Tukey posthoc test revealed that all concentrations 10 mg/L and higher moved significantly more (i.e. had higher mean distance) than the control larvae.

### Locomotion During Light Phases:

To assess differences in locomotion both within light phases and between them, we used a linear mixed-effects model (LMM) with the log-transformed individual mean distance moved (in 10 s bins) as the response variable, the interaction between time within the light phase and dosage as a fixed effect, interaction between dosage and light phase number as a fixed effect, and individual ID as a random effect. Both fixed effects were significant predictors of mean distance moved (LMM: Time*condition LRT=128.7, p<0.00, Condition*light.number LRT=547.4, p<0.001). This indicates the overall pattern that fraction 1 affects the rate of recovery within light events, as well as a potential effect of habituation to light numbers (or possible physical effects of longer term exposure). A Tukey posthoc pairwise comparison revealed that all dosages 10 mg/L and higher had a model estimate significantly different from the control.

For comparison with the rate of recovery (i.e. linear regression slope), we chose the 10 mg/L dosage, as this was the lowest dosage at which significant differences were found with the control within the overall model, but which did not have signs of long-term physical effects in the overall model. A two-sample t-test reveals that the 10 mg/L larvae have a significantly higher rate of recovery during light phases as compared to the DMSO control larvae (t=2.8, df=14.651, p-value=0.014).

### Fraction 2:

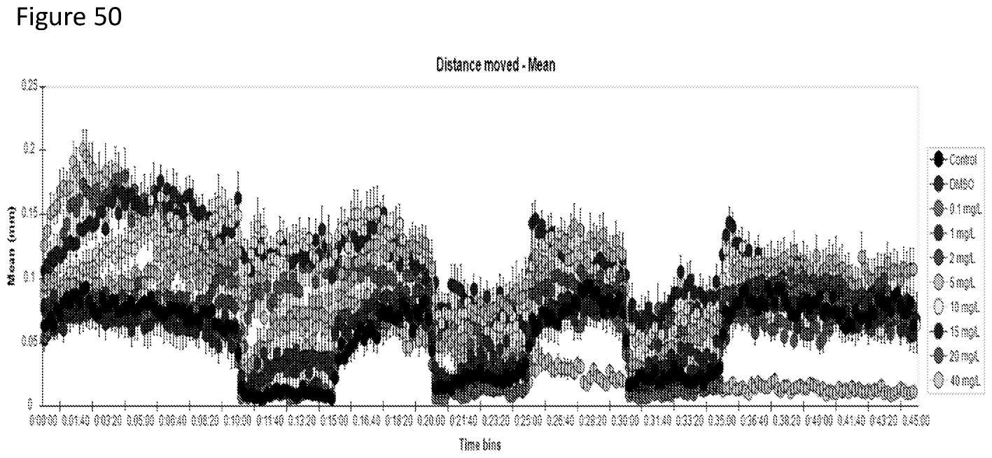

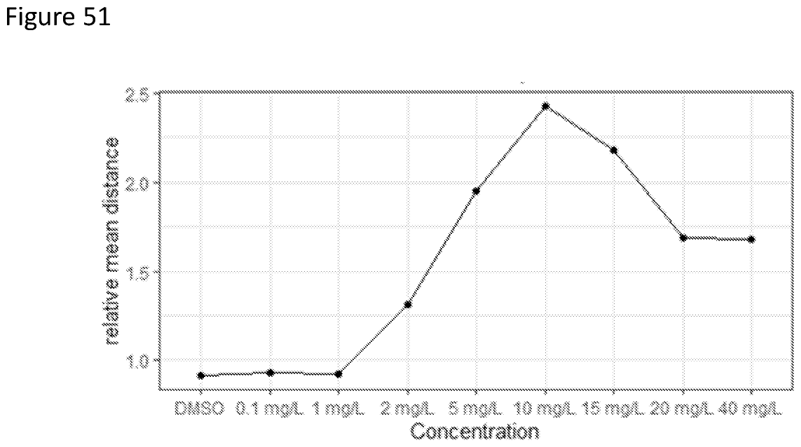

FIG. 50 shows the mean distance travelled (mm) per 10 seconds over the whole duration of the assay. Anything above 5 mg/L appears to be anxiolytic. Again a possible toxic effect at 40 mg/L. FIG. 51 shows the dose response curve of distance moved in the last minute of baseline.

### Exposure Effects:

To assess differences in locomotion after ˜10 minutes of exposure to the drug, we analysed mean distances moved over one minute. We used a linear mixed-effects model (LMM) with individual mean distance moved (in 10 s bins) as the response variable, dosage as a fixed effect, and individual ID as a random effect. Dosage was a significant predictor of mean distance moved after 10 minutes of exposure (LMM: LRT=64.4, p<0.001). A Tukey posthoc test revealed that all concentrations 10 mg/L (with an exception at 20 mg/L) and higher moved significantly more (i.e. had higher mean distance) than the control larvae. This is consistent with Fraction 1.

### Locomotion During Light Phases:

To assess differences in locomotion both within light phases and between them, we used a linear mixed-effects model (LMM) with the individual mean distance moved (in 10 s bins) as the response variable, the interaction between time within the light phase and dosage as a fixed effect, interaction between dosage and light phase number as a fixed effect, and individual ID as a random effect. Both fixed effects were significant predictors of mean distance moved (LMM: Time*condition LRT=152.1, p<0.00, Condition*light.number LRT=828.4, p<0.001). This indicates the overall pattern that fraction 2 affects the rate of recovery within light events, as well as a potential effect of habituation to light numbers (or possible physical effects of longer term exposure). A Tukey posthoc pairwise comparison revealed that all dosages 10 mg/L and higher had a model estimate significantly different from the control.

For comparison with the rate of recovery (i.e. linear regression slope), we chose the 10 mg/L dosage, as this was the lowest dosage at which significant differences were found with the control within the overall model, but which did not have signs of long-term physical effects in the overall model. A two-sample t-test reveals that that 10 mg/L dosage did not have a significantly higher rate of recovery than the DMSO control.

### Fraction 3:

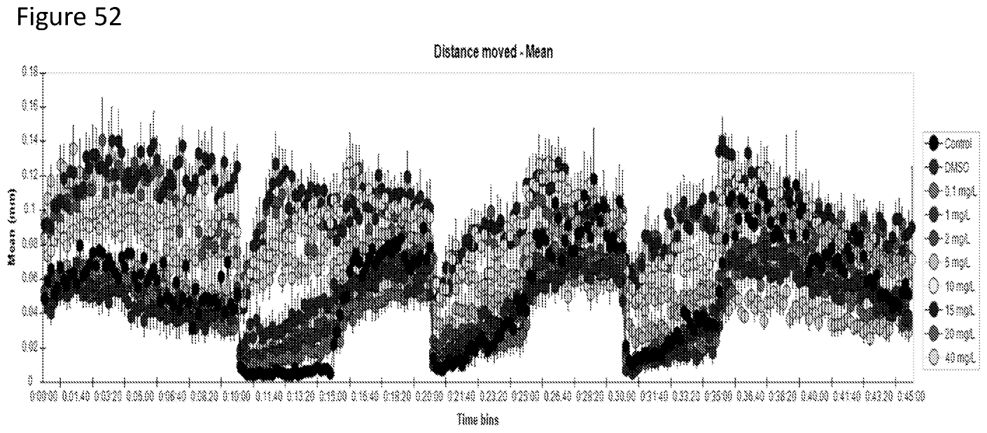

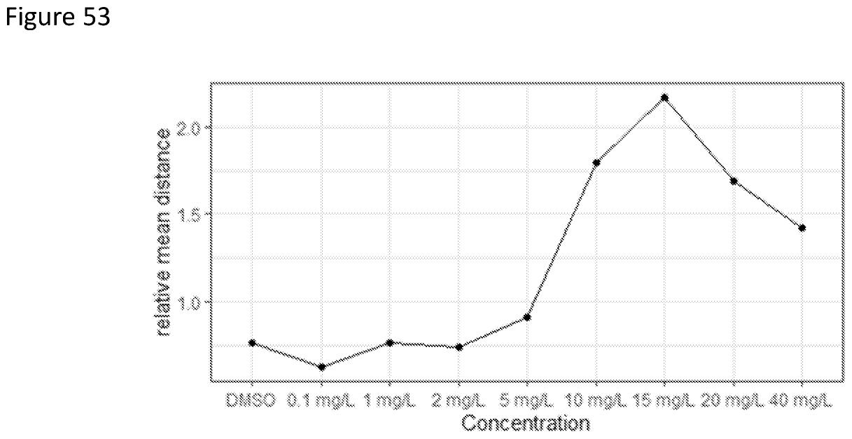

FIG. 52 shows the mean distance travelled (mm) per 10 seconds over the whole duration of the assay. Perhaps an effect starting at 1 mg/L, although the DMSO control seems to have an effect in the first period. However, there is an effect at 10 mg/L. 40 mg/L seems to be a toxic dose. FIG. 53 shows the dose response curve of distance moved in the last minute of baseline.

### Exposure Effects:

To assess differences in locomotion after ˜10 minutes of exposure to the drug, we analysed mean distances moved over one minute. We used a linear mixed-effects model (LMM) with individual mean distance moved (in 10 s bins) as the response variable, dosage as a fixed effect, and individual ID as a random effect. Dosage was a significant predictor of mean distance moved after 10 minutes of exposure (LMM: LRT=60.5, p<0.001). A Tukey posthoc test revealed that all concentrations 10 mg/L and higher moved significantly more (i.e. had higher mean distance) than the control larvae. This is consistent with Fractions 1 and 2.

### Locomotion During Light Phases:

To assess differences in locomotion both within light phases and between them, we used a linear mixed-effects model (LMM) with the individual mean distance moved (in 10 s bins) as the response variable, the interaction between time within the light phase and dosage as a fixed effect, interaction between dosage and light phase number as a fixed effect, and individual ID as a random effect. Both fixed effects were significant predictors of mean distance moved (LMM: Time*condition LRT=145.1, p<0.00, Condition*light.number LRT=288.4, p<0.001). This indicates the overall pattern that fraction 3 affects the rate of recovery within light events, as well as a potential effect of habituation to light numbers (or possible physical effects of longer term exposure). A Tukey posthoc pairwise comparison revealed that all dosages 10 mg/L and higher had a model estimate significantly different from the control.

For comparison with the rate of recovery (i.e. linear regression slope), we chose the 10 mg/L dosage, as this was the lowest dosage at which significant differences were found with the control within the overall model, but which did not have signs of long-term physical effects in the overall model. A two-sample t-test reveals that that 10 mg/L dosage did not have a significantly higher rate of recovery than the DMSO control.

### Test Product:

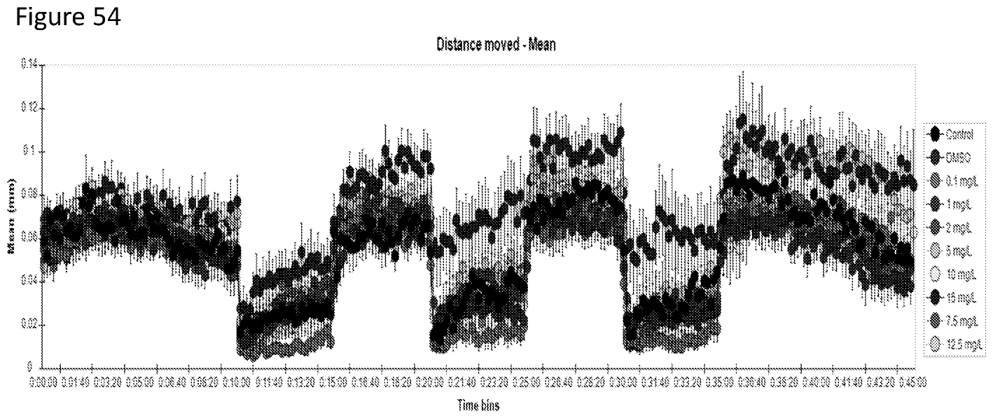

FIG. 54 shows the mean distance travelled (mm) per 10 seconds over the whole duration of the assay. Perhaps at the 5 mg/L dose but certainly at the 15 mg/L dose, there is an effect on movement. This is taken to indicate an anxiolytic effect. Overall, there appears to be an anxiolytic effect from the cannabinoid fractions, possibly increasing in efficacy from Fraction 1 to Fraction 3.

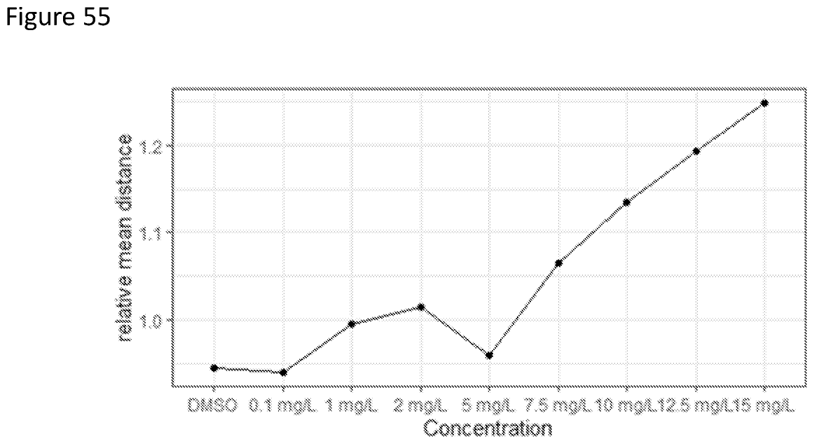

FIG. 55 shows the dose response curve of the distance moved in the last minute of baseline.

### Exposure Effects:

To assess differences in locomotion after ˜10 minutes of exposure to the drug, we analysed mean distances moved over one minute. We used a linear mixed-effects model (LMM) with individual mean distance moved (in 10 s bins) as the response variable, dosage as a fixed effect, and individual ID as a random effect. Dosage was not a significant predictor of distance moved (LMM: LRT=3.4, p=0.914). This implies that test product does not have a significant effect on locomotion in the absence of stress.

### Locomotion During Light Phases:

To assess differences in locomotion both within light phases and between them, we used a linear mixed-effects model (LMM) with the individual mean distance moved (in 10 s bins) as the response variable, HE interaction between time within the light phase and dosage as a fixed effect, interaction between dosage and light phase number as a fixed effect, and individual ID as a random effect. Both fixed effects were significant predictors of mean distance moved (LMM: Time*condition LRT=61.7, p<0.001, Condition*light.number LRT=152.3, p<0.001). However, a Tukey posthoc did not reveal significant pairwise differences between the DMSO control and any dosages.

Interestingly, the rate of recovery did not different during light events based on dosage (LMM, individual slope as response variable, light phase number and dosage as fixed effects, individual ID as random effect, LRT=7.4, p=0.594). However, condition on its own was a predictor of mean distance (LMM, LRT=17.2, p=0.046). This suggests that test product treated larvae did not recover faster because they did not freeze as much in the first place. Instead, they moved more at the beginning of the light phase and ‘recovered’ at the same rate.

### Comparison Between Fraction 1 and Test Product:

The differences in locomotion based on light phase in Fraction 1 and test product suggests that test product may have a different anxiolytic effect than Fraction 1. Larvae treated with Fraction 1 exhibited a similar pronounced stress response to DMSO larvae, but recovered more quickly. Larvae treated with test product do not exhibit as dramatic a stress response, but recover at a similar rate. Both seem consistent with possible anxiolytic effects and warrant further study.

### Example 6

### Methods

### Animal Husbandry:

Fish were bred and reared in the aquarium facility at Queen Mary University of London, licenced by the UK Home Office. Zebrafish (Danio rerio) adults from the Tuebingen wild type (TUWT) line were kept in glass breeding tanks in fish water that contain sodium bicarbonate (0.9 mM), calcium sulphate (0.05 mM) and marine salts (Sigma, Poole, UK; 0.018 g/l). Fish were maintained on a constant 14 h light: 10 h dark cycle at 28° C. They were fed 2 times a day with Zmsystems® ZM-000 high protein food particle (Tecniplast® UK. London) from 5 dpf-10 dpf, ZM-100 and paramecium from 11 dpf-14 dpf, and, ZM-200 and brineshrimp from 14 dpf-30 dpf. At one month of age, animals were transferred into the aquaria where they were fed Zmsystems® flake food and brineshrimp.

Zebrafish eggs were obtained by random mating between sexually mature individuals. The adult fish were introduced to a breeding tank with plastic plants and marbles preventing the adult fish from accessing and eating the eggs.

The following morning the eggs were harvested and kept at 28° C. and 50% humidity. The embryos were raised in 9 cm Petri dishes, approximately 60 per Petri dish in approximately 25 ml fishwater (water obtained from the adult housing tanks) at 28° C. The Petri dishes were cleaned by refreshing the eggwater and removing the dead embryos/larvae. This was done at 24-hour post fertilization (hpf).

### Forced Dark/Light Transition Assay:

5 Days post fertilization (dpf) larvae were incubated for 30 min with DMSO, 1% ethanol, 80 mg/L caffeine, 250 μg/L fluoxetine, 100 mM NaCl, or different concentrations of Fraction 1, Fraction 2 or the test product in a 48-well plate, one larva per well. After incubation the 48-well plate was placed in a DanioVision Observation Chamber (Noldus) and the larvae were subjected to a forced dark/light transition assay while still in the compound solution. The assay consisted of a 10 min baseline period in the dark, followed by a 10 min light challenge, followed by a 15 min dark period. DanioVision Observation Chamber controlled the lighting and recorded the assay with an infrared camera (resolution, 30 frames per second (fps)). Footage obtained was processed by EthoVision XT NUMBER (Noldus). Mean distance moved was measured with 10 second intervals.

| 30 min | 10 min | 10 min | 15 min |
|---|---|---|---|
| Acclimation + Incubation | Baseline | Light challenge | Dark Phase |

### Statistical Methodology:

All statistical analyses were carried out in R version 3.2.2 (R core developer team reference). Data distributions and model fit were initially assessed visually. AIC values were used in model selection. Due to difference in variance between groups we fitted a linear mixed model (LMM) in the gls package (ref).

It was first assessed whether the different compounds and concentrations were associated with differences in locomotion after 30 min exposure. We subsetted the last min in the baseline and fitted an LME with mean distance moved as the response variable, compound as the fixed effect, and individual as the random effect, with a variance structure for compound.

The light challenge and dark phase were subsetted to assess if different compounds and concentrations were associated with different patterns of movement. In the light challenge the increase of movement over time was considered recovery. In the dark phase decrease of movement was considered recovery. We fitted an LME with mean distance moved (in 10 second time bins) as the response variable, the interaction between time and concentration as a fixed effect, individual as the random effect and introduced a variant structure to account for the heterogeneity in variance between drugs.

### Results

### Baseline:

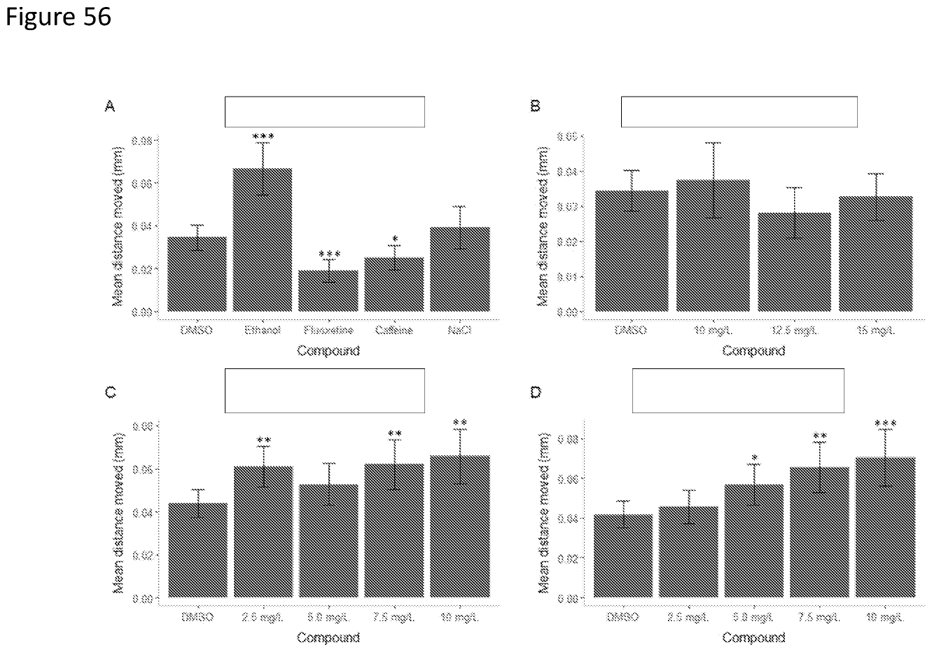

The baseline for different drugs and concentrations were compared to analyse differences in basal locomotion. Difference in locomotion were found between groups (p<0.001, FIG. 56). Fluoxetine and caffeine were found to move significantly less than DMSO (p<0.001, p<0.05). Ethanol however moved significantly more than DMSO (p<0.001). There was no difference in locomotion found with NaCl, see FIG. 56A. There were no significant effect found of the test product on locomotion in any of the tested concentrations, see FIG. 56B. All concentrations of Fraction 1, with the exception of 5.0 mg/L had a significantly higher basal movement (p<0.01), see FIG. 56C. There were significant differences found in basal movement in Fraction 2 concentrations 5.0 mg/L, 7.5 mg/L and 10 mg/L (p<0.05, p<0.01, p<0.001, respectively), see FIG. 56D.

### Light Challenge:

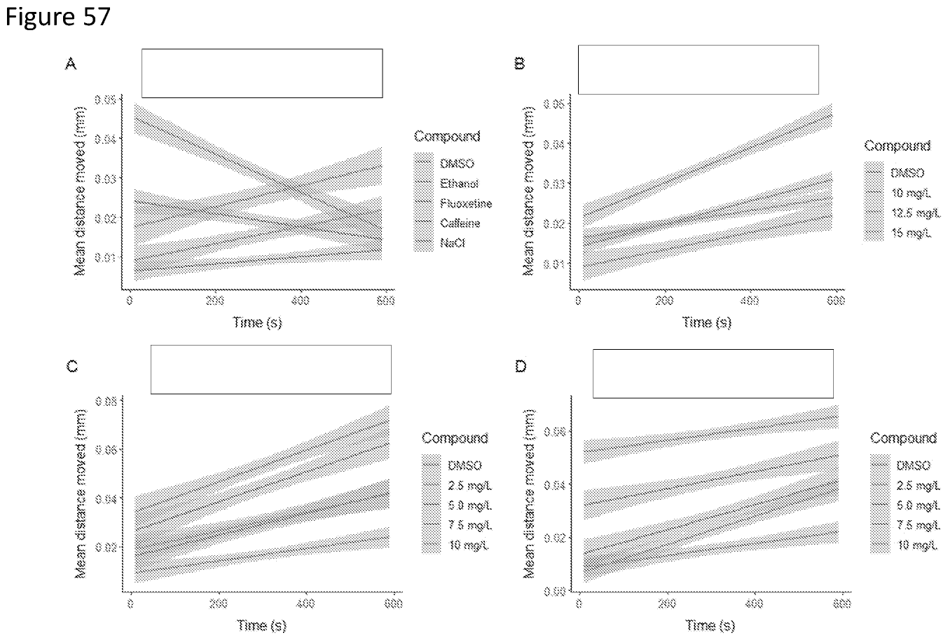

In the light challenge the rate of recovery was analysed and defined as an increase of movement over time. Ethanol and caffeine had a negative impact of locomotion over time (p<0.001); in addition they froze less in the beginning of the light challenge (p<0.001), while all other compounds were found to have a positive relation between movement and time. NaCl did not recover differently from DMSO in the light challenge (p>0.05). Fluoxetine was found to recover less over time (p<0.001), see FIG. 57A.

10 mg/L and 12.5 mg/L test product recover faster than DMSO (p<0.05), while 15 mg/L does not recover differently than DMSO (p>0.05). 10 mg/L and 15 mg/L do freeze less in the light compared to DMSO (p<0.05), see FIG. 57B.

All concentrations of fraction 1 recovered faster in the light challenge compared to the control group (2.5 mg/L p<0.01, 5.0 mg/L, 7.5 mg/L, 10 mg/L p<0.001). 2.5 mg/L, 7.5 mg/L and 10 mg/L were also found to freeze less in the light challenge (p<0.05, p<0.01, p<0.001, respectively), see FIG. 57C.

Concentrations 2.5 mg/L and 5.0 mg/L of Fraction 2 recover faster in the light challenge (p<0.001), 7.5 mg/L also recovers faster (p<0.05), while at the same time freezing less (p<0.001). The highest concentration 10 mg/L did not recover faster than DMSO (p>0.05), however they did freeze less (p<0.001), see FIG. 57D.

### Dark Phase:

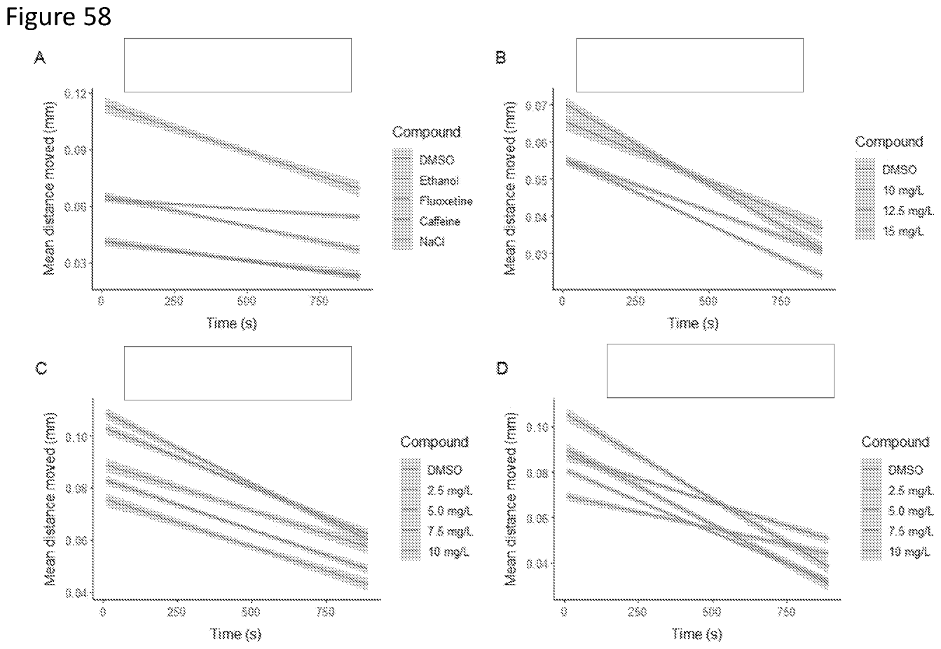

In the dark phase the rate of recovery was identified as the decrease of movement over time. All controls recover differently from DMSO (p<0.001), see FIG. 58A. Fluoxetine and caffeine recover less over time (p<0.001), they both also move less overall in the dark phase (p<0.001). NaCl recovers slower than DMSO (p<0.001), however there is no difference in movement in the beginning of the dark period (p>0.05). Ethanol recovers faster (p<0.001), however moves more at the beginning of the dark period compared to DMSO (p<0.001), see FIG. 58A.

10 mg/L test product recovers faster than DMSO (p<0.001). 12.5 mg/L test product does not recover differently from DMSO (p>0.05), while 15 mg/L test product recovers slower than DMSO (p<0.01). 12.5 mg/L and 15 mg/L move less in the beginning of the dark period (p<0.01). 10 mg/L does not have a difference in movement in the dark phase compared to DMSO (p>0.05), see FIG. 58B.

2.5 mg/L and 5.0 mg/L of fraction 1 do not recover faster in the dark than DMSO (p>0.05), while concentrations 7.5 mg/L and 10 mg/L recover faster than DMSO (p<0.001). 2.5 mg/L of fraction 1 moves more in the beginning of the dark period (p<0.01), and so do 7.5 mg/L and 10 mg/L of fraction 1 (p<0.001), see FIG. 58C.

All concentrations of fraction 2 recover faster than DMSO in the dark (p<0.001). All concentrations of fraction 2 also moved more in the beginning of the dark period (2.5 mg/L p<0.01), 5.0 mg/L, 7.5 mg/L, 10 mg/L p<0.001), FIG. 58D.

Conclusion: Fraction 1, Fraction 2 and the test product have an anxiolytic effect, as shown by a faster rate of recovery in the light challenge and dark phase.

### Example 7

Extraction of Hemp Fibre Pellets with Supercritical CO₂:

Dungaro hemp pellets were extracted with a bespoke supercritical CO₂ rig comprised of two extraction vessels with a combined working volume of 10 litres. Extraction pressure was 180 bar, temperature 40° C. and density 816.1.

Fractions were collected under the following conditions:
Fraction collector 1) 120 bar, 50° C., density 510.6-green fraction, contains chlorophyll, lipids and cannabinoids;
Fraction collector 2) 80 bar, 40° C., density 219-yellow fraction, contains cannabinoids Fraction collector 3) 50 bar, 25° C., density 113-oil fraction, contains terpenes and cannabinoids.
Fraction weights, 1) 19.3%, 2) 77.5%, 3) 3.21%

### Preparation for Analysis:

Weighed 10 mg of extract and added to 5 mL 100% methanol and placed on a shaker for 1 hr. Samples were centrifuged for 15 min at 10,000 g and the supernatant was transferred to a clean container. The pellet was re-extracted with 5 mL 100% methanol and centrifuged as above. The supernatant was combined with the first supernatant fraction and this total volume was dried down in a vacuum centrifugal drier. The dried residue was resuspended in 100 μL, diluted 1:10 and analysed by LC-PDA-MSn as described below.

The analysis was carried out with a Thermo Finnigan LTQ MS® system (Thermo Electron Corporation, USA) comprising a Finnigan Surveyor PDA Plus® detector, a Finnigan LTQ® linear ion trap with ESI source and a Waters C18Nova-Pak column (60 Å, 4 μm, 3.9 mm×150 mm; WAT036975) (with guard column fitted) and autosampler.

### Instrument Conditions:

The instrument is operated via the Xcalibur® software programme, the following conditions are set in the method file—
Autosampler: The sample tray is maintained at 5° C. The column is maintained at 30° C. Injection volume 10/20 μL
Pump: LC solvents: A: water with 0.1% formic acid; B: methanol with 0.1% formic acid, flow rate: 1 mL/min; gradient: 60-100% B in 20 min. Wash step: 5 min 100% B, 10 min 60% B.
PDA: scan range 240-400 nm
Mass Spectrometer conditions: tune file: chlorogenic0608. Sheath gas 30 (arbitrary units), auxillary gas 15, sweep gas 0, capillary Temperature 320° C., spray voltage −4.0 kV/+4.8 kV, capillary voltage −1V/+45V, tube lens −68V/+110V.
Mass Spectrometer detection: scan events:
   Run 1:
   PDA: wavelength range 240-400 nm
   MS:
   Full MS positive mode,
   MS2 357.00@cid35.00 [95.00-360.00] in negative mode (targeting CBDA)
   MS2 315.00@cid35.00 [85.00-320.00] in positive mode (targeting THC and CBD)
   Run 2:
   PDA: wavelength range 240-400 nm
   MS:
   Full MS positive mode,
   Data-dependent scan targeting most abundant ion.

Data files analysed using Qual Browser in Xcalibur®.

### Results

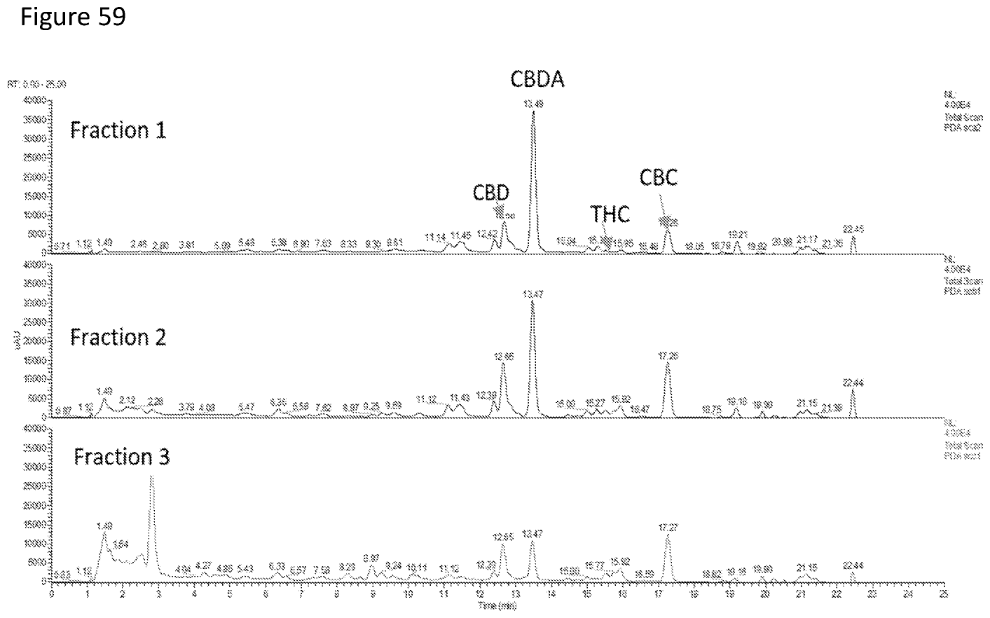

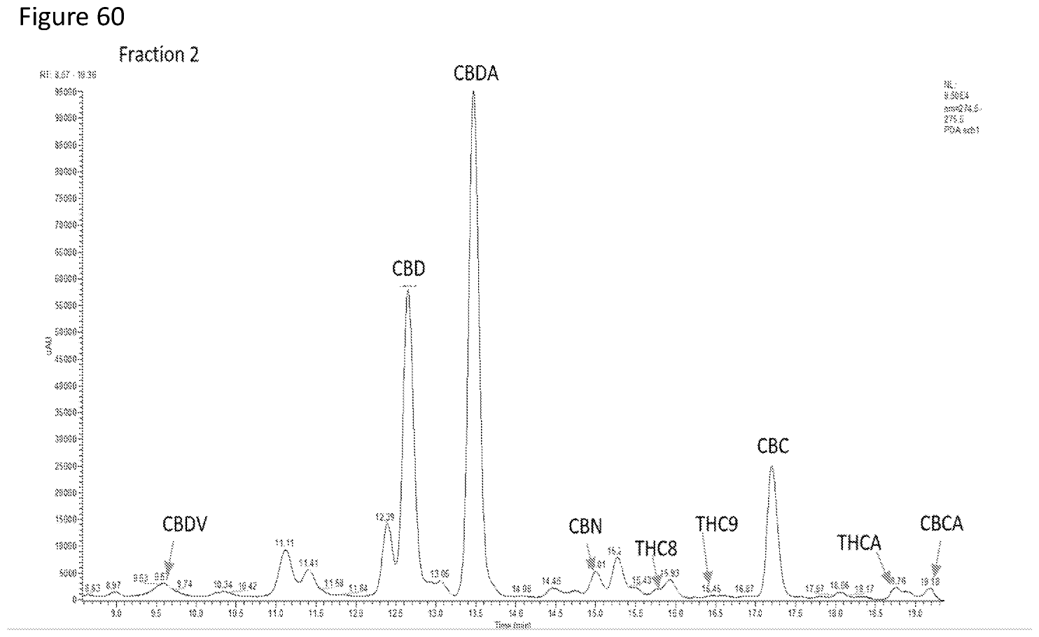

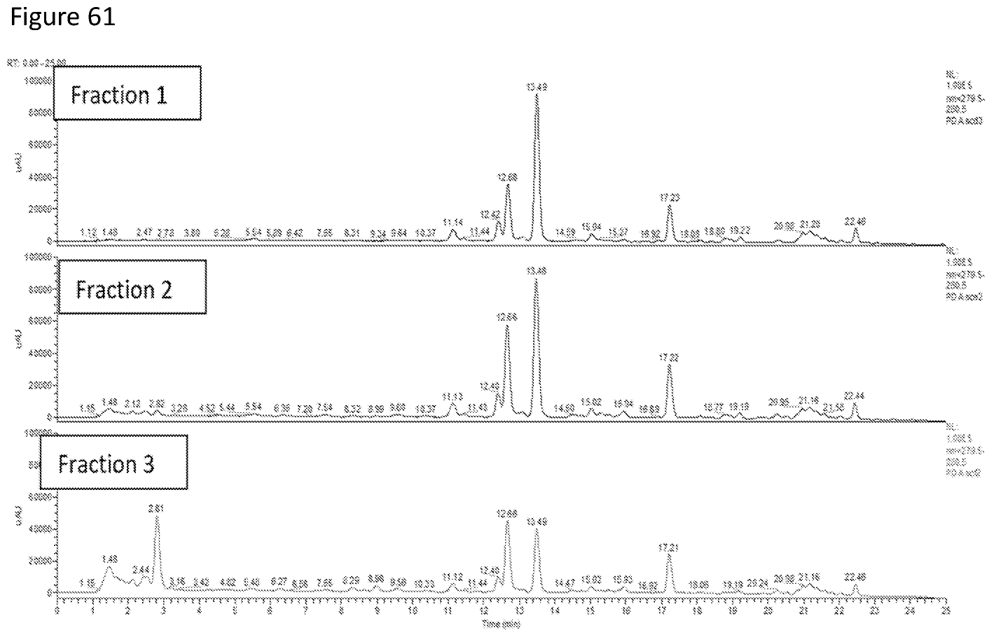

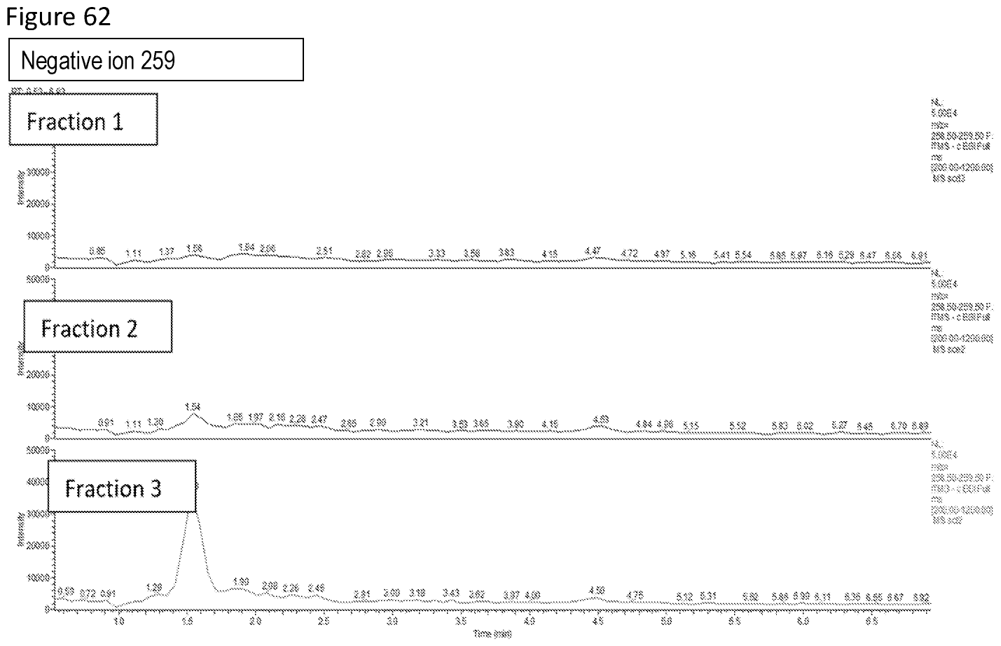

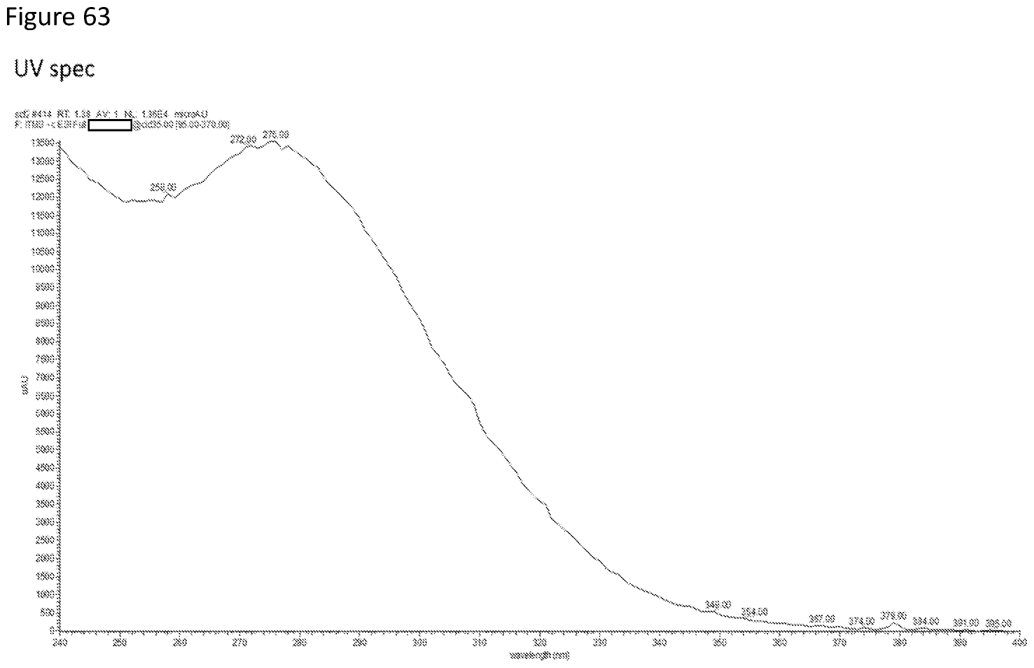

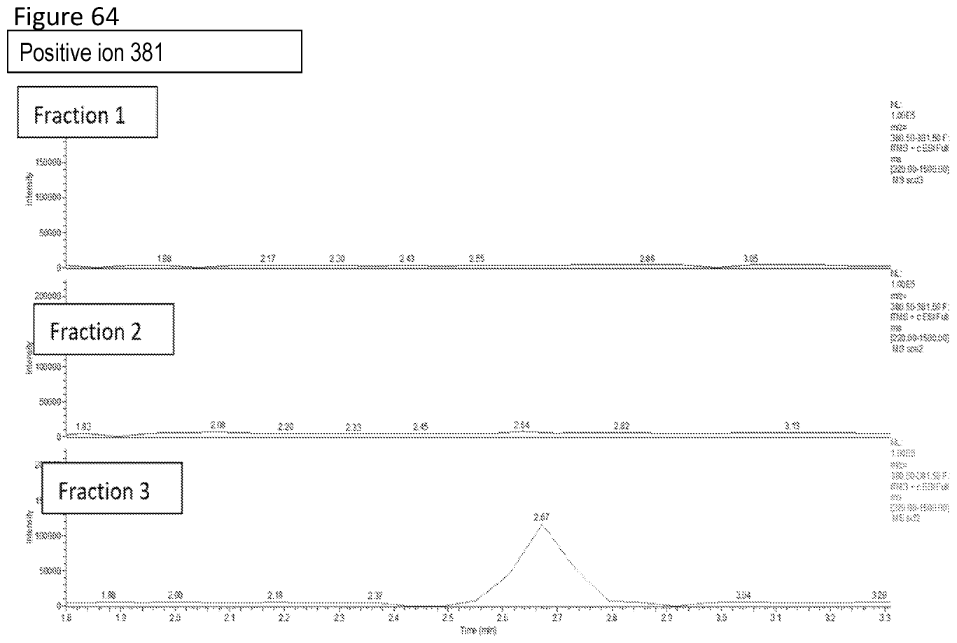

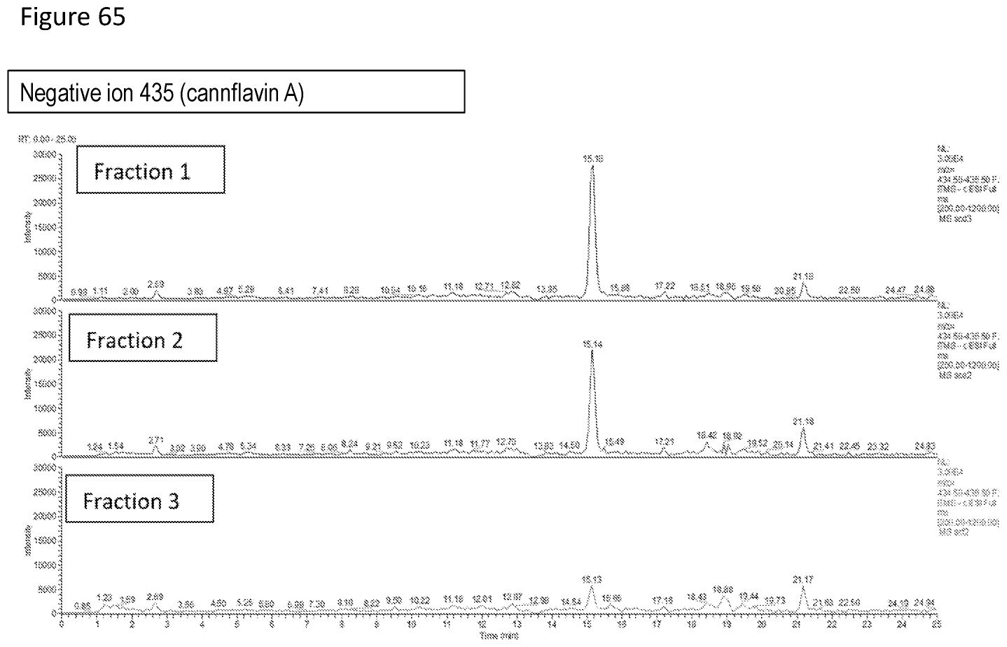

FIGS. 59 to 65 show chromatograms showing the analysis of the different fractions.

Fraction 1 contained lipids and chlorophylls and fraction 3 contained terpenes. All fractions contained cannabinoids but most material went into fraction 2 (77.5% of total product weight). A relatively high content of CBC was also detected in these fractions.

Table 1 below summarizes the cannabinoid content of the fractions.

% Cannabinoids in Supercritical CO₂ fractions

| | Fraction 1 | Fraction 2 | Fraction 3 |
|---|---|---|---|
| CBDV | 0.430 | 0.427 | 0.447 |
| CBD | 7.299 | 9.795 | 8.676 |
| CBDA | 2.128 | 1.704 | 0.934 |
| CBN | 0.055 | 0.069 | 0.038 |
| THC8 | 0.257 | 0.240 | 0.162 |
| THC9 | 0.185 | 0.171 | 0.131 |
| CBC | 0.451 | 0.507 | 0.376 |
| THCA | 0.041 | 0.040 | 0.021 |
| CBCA | 0.024 | 0.018 | 0.009 |

### Example 8

Tank diving experiments were performed as follows:

Fish were exposed to drug, either pure CBD, CBD containing terpenes, or CBD plus CBG, in a litre of water for 30 min prior to being transferred to a novel tank containing fresh fish water in the absence of drug using a net and their behaviour recorded over a 6 min period using a camera mounted to one side. The vertical position of the fish in the tank can reliably be used as a proxy for an individual's anxiety. For example, the less time a fish spends on the bottom of the tank the less anxious. The response variables were number of visits to the top half of the tank, distance from the bottom of the tank, and proportion of time spent in the bottom third of the tank. Time spent on the bottom, distance travelled and number of top half visits determining using Ethovision software (Noldus). Water was changed, tanks and fish nets cleaned between each fish.

The anxiolytic concentration of CBD was first assessed by treating the fish with 0-15 mg/L CBD.

The effect of terpenes on the anxiolytic effect of CBD was then assessed by treating the fish with different proportions of CBD with 0-2 mg/L test compound, which contains both CBD and terpenes, so that CBD concentration was held constant at 2 mg/L whilst the concentration of terpenes increased. The ratio of CBD minus terpenes:CBD plus terpenes tested was 2:0, 1.5:0.5, 1:1, 0.5:1.5 and 0:2.

Finally, the anxiolytic effect of CBG was assessed by treating the fish with 2 mg/L CBD and 0-5 mg/L CBG.

### Result

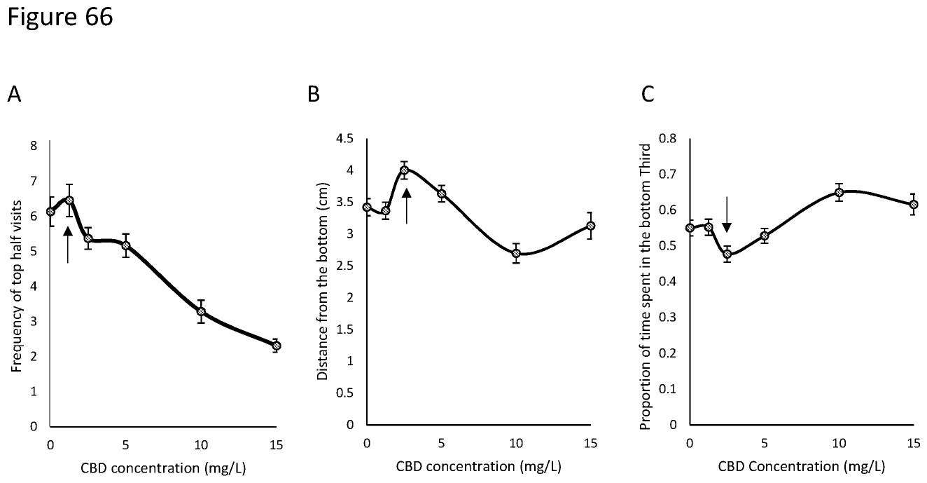

FIG. 66 shows the anxiolytic effect of 0-15 mg/L CBD on (A) the frequency of visits to the top half of the tank (more top half visits indicates a reduction in anxiety), (B) distance from the bottom of the tank (greater distance from the bottom indicates a reduction in anxiety), and (C) the proportion of time spent in the bottom third of the tank (less time spent in the bottom third of the tank indicates a reduction in anxiety). The greatest anxiolytic effect is marked by an arrow.

FIG. 67 shows the anxiolytic effect of a mixture of CBD and 0-2 mg/L test compound which contains CBD and terpenes, so that CBD concentration was held constant at 2 mg/L whilst the concentration of terpenes increased, on (A) the frequency of visits to the top half of the tank, (B) distance from the bottom of the tank, and (C) the proportion of time spent in the bottom third of the tank. The greatest anxiolytic effect is marked by an arrow. These results show that the anxiolytic effect of the compositions was increased when the compositions contained a terpene.

FIG. 68 shows the anxiolytic effect of 2 mg/L CBD in combination with 0-5 mg/L CBG on (A) the frequency of visits to the top half of the tank, (B) distance from the bottom of the tank, and (C) the proportion of time spent in the bottom third of the tank. These results show that the inclusion of CBG in the composition caused the fish to visit the top half of the tank with decreased frequency (FIG. 68A), and to spend a greater proportion of time in the bottom third of the tank (FIG. 68C). FIG. 68B shows no significant change in the distance from the bottom of the tank with increasing CBG concentration. In particular, FIG. 68A shows that the frequency of visits to the top half of the tank decreased with increasing concentration of CBG. Thus, the results demonstrate that the presence of CBG in the mixture displays no anxiolytic effect, but may potentially have an undesirable anxiogenic effect.

1. A composition comprising a cannabinoid and at least one terpene, wherein the cannabinoid comprises cannabidiol (CBD), the terpene:cannabinoid ratio by weight is from 1:3 to 1:60 w/w and the composition in total comprises ≤0.001% w/w of 9-tetrahydrocannabinol (THC) and <0.1% w/w of cannabigerol (CBG).

2. The composition according to claim 1, wherein CBG is at a concentration of ≤0.01% w/w, or preferably at a concentration of ≤0.001% w/w.

3. The composition according to claim 1, wherein the composition does not comprise CBG.

4. The composition according to claim 1, wherein the at least one terpene comprises alpha-humulene, beta-caryophyllene, alpha-pinene, beta-pinene, terpinolene, beta-myrcene and/or an ocimene isomer.

5. The composition according to claim 4, wherein the at least one terpene comprises one or more of terpinolene, beta myrcene and beta pinene.

6. The composition according to claim 1, wherein the cannabinoid is present at a concentration of 0.03-30% w/w, preferably 1-25% w/w, more preferably 5-25% w/w.

7. The composition according to claim 1, wherein the at least one terpene is present at a concentration of 0.01-10% w/w, preferably 0.1-6% w/w, more preferably 0.5-3% w/w.

8. The composition according to claim 1, wherein the composition comprises ≤0.0001% w/w of cannabinol (CBN), 9-tetrahydrocannabinol (THC), and/or 9-tetrahydrocannabinolic acid (THC-A).

9. The composition according to claim 8, wherein the composition comprises ≤0.0001% w/w of cannabichromene (CBC) and cannabidiolic acid (CBD-A).

10. The composition according to claim 1, wherein the composition does not comprise THC.

11. The composition according to claim 1, wherein the composition further comprises an oil.

12. The composition according to claim 11, wherein the cannabinoid:oil ratio is 1:2 to 1:3500 w/w.

13. The composition according to claim 11, wherein the oil is present at 60-99.96% w/w.

14. The composition according to claim 11, wherein the oil is selected from hemp seed oil, rape seed oil, coconut oil, olive oil, cranberry oil, vegetable oil and MCT oil, or a mixture or two or more oils selected from hemp seed oil, rape seed oil, coconut oil, olive oil, cranberry oil and MCT oil.

15. A pharmaceutical formulation comprising the composition according to claim 1 and a pharmaceutically-acceptable diluent, carrier or excipient.

16. A method of treatment or prophylaxis comprising administering the composition of claim 1 to a subject in need thereof.

17. The method of treatment or prophylaxis according to claim 16, wherein the treatment or prophylaxis is of anxiety or a disorder associated with anxiety.

18. The method of treatment or prophylaxis according to claim 17, wherein the disorder associated with anxiety is selected from any one of generalized anxiety disorder, chronic anxiety, social anxiety disorder, panic disorder or episodic paroxysmal anxiety, obsessive compulsive disorder, post-traumatic stress disorder, phobic anxiety disorders, social phobias, specific phobias such as agoraphobia, claustrophobia or animal phobias, acute stress disorder, separation anxiety disorder, selective mutism, substance or medication-induced anxiety disorder, anxiety disorders due to other medical conditions, anxiety disorders without a specific cause, depression, atypical depression, recurring subclinical anxiety, persistent anxiety, chronic subclinical anxiety, persistent anxiety, anxious depression, neurosis, healing avoidance anxiety, dissociative anxiety, mixed anxiety and depressive disorder, severe stress, adjustment disorders, dissociative disorders such as dissociative amnesia, dissociative fugue, dissociative stupor, trance and possession disorders, dissociative motor disorders, dissociative convulsions, dissociative anaesthesia and sensory loss, mixed dissociative disorders, Ganser syndrome, multiple personality disorder and dissociative conversion disorder, somatoform disorders such as somatization disorder, undifferentiated somatoform disorder, hypochondriacal disorder, somatoform autonomic dysfunction and persistent somatoform pain disorder, and neurotic disorders such as neurasthenia, depersonalization-derealization syndrome, Dhat syndrome, occupational neurosis, psychasthenia, psychasthenic neurosis and psychogenic syncope.

19. A food or beverage product comprising a composition according to claim 1.

20. The method of treatment or prophylaxis according to claim 17, wherein the anxiety is non-clinical anxiety.
